(19) United States
(12) Patent Application Publication
(10) Pub. No.: US 2021/0220324 A1
(43) Pub. Date: Jul. 22, 2021

(54) COMPOSITIONS COMPRISING COMBINATIONS OF PURIFIED CANNABINOIDS, WITH AT LEAST ONE FLAVONOID, TERPENE, OR MINERAL

(71) Applicant: Canopy Growth Corporation, Smiths Falls (CA)

(72) Inventors: Kurt Aron Levy, Roswell, GA (US); Jonathan Michael Cooper, Denver, CO (US)

(21) Appl. No.: 17/174,044

(22) Filed: Feb. 11, 2021

Related U.S. Application Data
(63) Continuation of application No. 15/616,874, filed on Jun. 7, 2017.
Application No. 15/616,874 is a continuation-in-part of application No. 15/055,499, filed on Feb. 26, 2016, now abandoned.
(60) Provisional application No. 62/126,365, filed on Feb. 27, 2015.
(60) Provisional application No. 62/347,558, filed on Jun. 8, 2016.
(60) Provisional application No. 62/348,114, filed on Jun. 9, 2016.
(60) Provisional application No. 62/349,296, filed on Jun. 13, 2016.
(60) Provisional application No. 62/369,932, filed on Aug. 2, 2016.
(60) Provisional application No. 62/354,463, filed on Jun. 24, 2016.
(60) Provisional application No. 62/465,688, filed on Mar. 1, 2017.
(60) Provisional application No. 62/469,415, filed on Mar. 9, 2017.
(60) Provisional application No. 62/475,153, filed on Mar. 22, 2017.
(60) Provisional application No. 62/477,990, filed on Mar. 28, 2017.
(60) Provisional application No. 62/489,360, filed on Apr. 24, 2017.
(60) Provisional application No. 62/490,567, filed on Apr. 26, 2017.
(60) Provisional application No. 62/490,465, filed on Apr. 26, 2017.
(60) Provisional application No. 62/491,160, filed on Apr. 27, 2017.
(60) Provisional application No. 62/491,175, filed on Apr. 27, 2017.
(60) Provisional application No. 62/491,899, filed on Apr. 28, 2017.
(60) Provisional application No. 62/491,980, filed on Apr. 28, 2017.
(60) Provisional application No. 62/508,129, filed on May 18, 2017.
(60) Provisional application No. 62/509,651, filed on May 22, 2017.
(60) Provisional application No. 62/510,229, filed on May 23, 2017.
(60) Provisional application No. 62/510,660, filed on May 24, 2017.
(60) Provisional application No. 62/510,672, filed on May 24, 2017.
(60) Provisional application No. 62/511,218, filed on May 25, 2017.

(51) Int. Cl. A61K 31/352; A61K 31/01; A61K 31/05; A61K 31/015; A61K 31/045; A61K 31/122; A61K 31/35; A61K 36/185; A61K 31/353; A61K 33/00

(52) U.S. Cl. CPC A61K 31/352; A61K 31/01; A61K 31/05; A61K 31/015; A61K 33/00; A61K 31/122; A61K 31/35; A61K 36/185; A61K 31/353; A61K 31/045

(57) ABSTRACT

Disclosed herein are new compositions having combinations of purified cannabinoids. One embodiment of this disclosure provides compositions having one or more purified cannabinoids in combination with a purified terpene. One embodiment of this disclosure provides compositions having one or more purified cannabinoids in combination with a purified flavonoid. One embodiment of this disclosure provides compositions having one or more purified cannabinoids in combination with a purified mineral.

### CROSS REFERENCE TO RELATED APPLICATIONS

This application is a continuation of U.S. patent application Ser. No. 15/616,874, filed on Jun. 7, 2017, which is a continuation in part of U.S. patent application Ser. No. 15/055,499, filed on Feb. 26, 2016, which claims priority to U.S. Provisional Patent Application Ser. No. 62/126,365, filed on Feb. 27, 2015, which is hereby incorporated by reference. U.S. patent application Ser. No. 15/616,874 also claims priority to U.S. Provisional Patent Application Ser. No. 62/347,558, filed on Jun. 8, 2016, U.S. Provisional Patent Application Ser. No. 62/348,114, filed on Jun. 9, 2016, U.S. Provisional Patent Application Ser. No. 62/349,296 filed on Jun. 13, 2016, U.S. Provisional Patent Application Ser. No. 62/369,932 filed on Aug. 2, 2016, U.S. Provisional Patent Application Ser. No. 62/354,463 filed on Jun. 24, 2016, U.S. Provisional Patent Application Ser. No. 62/465,688 filed on Mar. 1, 2017, U.S. Provisional patent Application Ser. No. 62/469,415 filed on Mar. 9, 2017, U.S. Provisional Patent Application Ser. No. 62/475,153 filed on Mar. 22, 2017, U.S. Provisional Patent Application Ser. No. 62/477,990 filed on Mar. 28, 2017, U.S. Provisional Patent Application Ser. No. 62/489,360 filed on Apr. 24, 2017, U.S. Provisional Patent Application Ser. No. 62/490,465 filed on Apr. 26, 2017, U.S. Provisional Patent Application Ser. No. 62/490,567 filed on Apr. 26, 2017, U.S. Provisional Patent Application Ser. No. 62/491,160 filed on Apr. 27, 2017, U.S. Provisional Patent Application Ser. No. 62/491,175 filed on Apr. 27, 2017, U.S. Provisional Patent Application Ser. No. 62/491,899 filed on Apr. 28, 2017, U.S. Provisional Patent Application Ser. No. 62/491,980 filed on Apr. 28, 2017, U.S. Provisional Patent Application Ser. No. 62/508,129 filed on May 18, 2017, U.S. Provisional Patent Application Ser. No. 62/509,651 filed on May 22, 2017, U.S. Provisional patent Application Ser. No. 62/510,229 filed on May 23, 2017, U.S. Provisional Patent Application Ser. No. 62/510,660 filed on May 24, 2017, U.S. Provisional Patent Application Ser. No. 62/510,672 filed on May 24, 2017, and U.S. Provisional Patent Application Ser. No. 62/511,218 filed on May 25, 2017, each of which is hereby incorporated by reference.

### TECHNICAL FIELD

This disclosure relates to the cannabis industry. In particular, this disclosure relates to compositions and formulations comprising purified cannabinoids, flavonoids, and/or terpenes.

### BACKGROUND

The word “cannabis” refers to a genus of flowering plants. Plants of genus cannabis include several species, including Cannabis sativa, Cannabis indica, and Cannabis ruderalis. There is a long history of cultivating plants of genus cannabis for hemp fibers, seeds and seed oils, medicinal purposes, and recreational activities.

According to some accounts, cannabis is composed of at least 483 known chemical compounds, which include cannabinoids, terpenoids, flavonoids, nitrogenous compounds, amino acids, proteins, glycoproteins, enzymes, sugars and related compounds, hydrocarbons, alcohols, aldehydes, ketones, acids, fatty acids, esters, lactones, steroids, terpenes, non-cannabinoid phenols, vitamins, and pigments.

Cannabinoids are of particular interest for research and commercialization. Most extractions of cannabis plant matter aim to extract cannabinoids, particularly tetrahydrocannabinol (THC). THC is useful for relieving pain, treating glaucoma, and relieving nausea. THC is also gaining immense popularity as a recreational drug substance. Usually, cannabinoids are extracted from the cannabis plant as part of a crude mixture, combined with other chemical compounds found in the cannabis plant.

Despite developments in isolating single molecules, no work has been done in formulating compositions having particular combinations of the purified compounds. There has been no work developing compounds having repeatable, dependable ratios of purified molecules. In particular, there have been no developments in combining purified cannabinoids with purified terpenes, flavonoids, and/or minerals.

Despite developments in isolating a handful of the cannabinoids and terpenes found in cannabis plants, no work has been done in formulating compositions having particular combinations containing purified or isolated forms of these compounds. Some of the less common cannabinoids have neither been isolated nor studied alone or in any combination. Additionally, there has been little or no work developing compositions having purposefully engineered, repeatable, consistent, and dependable ratios of purified cannabinoids. In particular, there have been no developments in combining a cannabinoid with a purified terpene.

There exists a need for compositions comprising new combinations of purified cannabinoids. There also exists a particular need for compositions providing one or more purified cannabinoids in combination with a purified terpene. Furthermore, there exists a need for compositions providing one or more purified cannabinoids in combination with a purified flavonoid. Additionally, there exists a need for compositions providing one or more purified cannabinoids in combination with a purified mineral.

There exists a need for compositions comprising new man-made combinations of purified cannabis components. In particular, there exists a need for compositions comprising new combinations of at least one cannabinoid and at least one purified terpene.

### DETAILED DESCRIPTION

Disclosed herein are new compositions having combinations of purified cannabinoids. One embodiment of this disclosure provides compositions having one or more purified cannabinoids in combination with a purified terpene. One embodiment of this disclosure provides compositions having one or more purified cannabinoids in combination with a purified flavonoid. One embodiment of this disclosure provides compositions having one or more purified cannabinoids in combination with a purified mineral.

Disclosed herein are new compositions comprising purified cannabinoids and/or purified terpenes. In one embodiment of this disclosure, the compositions comprise a first purified cannabinoid and a first purified terpene. In one embodiment of this disclosure, the compositions comprise a first purified cannabinoid and a second purified cannabinoid. In one embodiment of this disclosure, the compositions comprise a first purified cannabinoid, a second purified cannabinoid, and a first purified terpene. In one embodiment of this disclosure, the compositions comprise a first purified cannabinoid, a first purified terpene, and a second purified terpene.

The compositions disclosed herein are purposefully formulated to provide man-made, non-naturally occurring combinations, concentrations, and/or ratios of compounds sometimes found in naturally occurring cannabis plants.

In one embodiment, the compositions disclosed herein include a non-naturally occurring ratio of a first purified cannabinoid and a first purified terpene. In one embodiment, the compositions disclosed herein include a non-naturally occurring concentration of a first purified cannabinoid and a first purified terpene.

In one embodiment, the compositions disclosed herein include a non-naturally occurring ratio of a first purified cannabinoid and a second purified cannabinoid. In one embodiment, the compositions disclosed herein include a non-naturally occurring concentration of a first purified cannabinoid and a second purified cannabinoid.

In one embodiment, the compositions disclosed herein include a non-naturally occurring ratio of a first purified cannabinoid, a second purified cannabinoid, and a first purified terpene. In one embodiment, the compositions disclosed herein include a non-naturally occurring concentration of a first purified cannabinoid, a second purified cannabinoid, and a first purified terpene.

In one embodiment, the compositions disclosed herein include a non-naturally occurring ratio of a first purified cannabinoid, a second purified cannabinoid, and a third purified cannabinoid. In one embodiment, the compositions disclosed herein include a non-naturally occurring concentration of a first purified cannabinoid, a second purified cannabinoid, and a third purified cannabinoid.

In one embodiment, the compositions disclosed herein include a non-naturally occurring ratio of a first purified terpene, a second purified terpene, and a first purified cannabinoid. In one embodiment, the compositions disclosed herein include a non-naturally occurring concentration of a first purified terpene, a second purified terpene, and a first purified cannabinoid.

In one embodiment of this disclosure, the compositions comprise an unnaturally occurring ratio of 10:1-1:1 of a purified cannabinoid to purified terpene.

In one embodiment of this disclosure, the compositions comprise an unnaturally occurring ratio of 10:1-5:1 of a purified cannabinoid to purified terpene.

In one embodiment of this disclosure, the compositions comprise an unnaturally occurring ratio of greater than 10:1 of a purified cannabinoid to purified terpene.

Disclosed herein are compositions comprising:
a first purified cannabinoid; and
a compound chosen from a second purified cannabinoid, a purified terpene, a purified flavonoid, and a purified mineral,
wherein the said composition is substantially free from cellulose.

Disclosed herein are compositions comprising:
a first purified cannabinoid; and
a compound chosen from a second purified cannabinoid or a first purified terpene,
wherein the said composition is substantially free from cellulose.

As used within the context of this application, the term “purified” means extracted, isolated, and/or separated from other compounds, formulations, compositions, matter, and/or mass. In one embodiment, the term “purified” refers to a cannabinoid that is separated from the plant matter from which it was derived.

In one embodiment, as used herein, the term “purified” means isolated from the plant using chromatography, distillation, extractions, or similar technique resulting in a greater than 60% purity. In some embodiments the “purified” compositions disclosed herein are greater than 70% purity. In some embodiments the “purified” compositions disclosed herein are greater than 80% purity. In some embodiments the “purified” compositions disclosed herein are greater than 90% purity.

In one embodiment, the term “purified” refers to a cannabinoid (a “purified cannabinoid”) that is separated from other cannabinoids that were present in the plant matter from which it was derived. In one embodiment, the term “purified” refers to a cannabinoid (a “purified cannabinoid”) that is separated from terpenes that were present in the plant matter from which it was derived. In one embodiment, the term “purified” refers to a cannabinoid (a “purified cannabinoid”) that is separated from secondary compounds that were present in the plant matter from which it was derived. In one embodiment, the term “purified” refers to a cannabinoid (a “purified cannabinoid”) that is separated from all material that was present in the plant matter from which it was derived.

In one embodiment, the term “purified” refers to a terpene (a “purified terpene”) that is separated from other cannabinoids that were present in the plant matter from which it was derived. In one embodiment, the term “purified” refers to a terpene (a “purified terpene”) that is separated from terpenes that were present in the plant matter from which it was derived. In one embodiment, the term “purified” refers to a terpene (a “purified terpene”) that is separated from secondary compounds that were present in the plant matter from which it was derived. In one embodiment, the term “purified” refers to a terpene (a “purified terpene”) that is separated from all material that was present in the plant matter from which it was derived.

Within the context of this disclosure, purified compounds may be purposely formulated with other compounds at various levels of purity. For example, depending on the desired outcome, a particular cannabinoid or terpene may be formulated with other molecules when it is 60-65% pure, 65-70% pure, 70-75% pure, 75-80% pure, 80-85% pure, 85-90% pure, 90-95% pure, 95-99% pure, 99-99.9% pure, 99.9+%, or greater than 99% pure. Provided that the ingredients used for purposeful formulation are purified prior to the said purposeful formulation, the act of subsequently formulating them does render them not “purified” within the context of an ingredient list.

In one embodiment, purified means “substantially free” from other material, e.g., compounds, particles, vegetative material, plant derived substances, solvents, etc. In one example, the term “purified” refers to a compound purified from a crude extract, such as a biologically derived substance or BDS, thereby resulting in a significant difference between the purified compound and the extract. In one embodiment, substantially free means that the compound comprises no (or insignificant amounts) of other materials.

Within the context of this disclosure, the term “plant” refers to the whole plant and parts of the plant, e.g., leaves, roots, bark, flower, etc. The term plant also encompasses a dried plant and dried parts of the plant.

Within the context of this disclosure, where a compound comprises stereogenic centers, the term “purified” includes isolated stereoisomers and also mixtures of stereoisomers, provided that the compound having the stereoisomers is free from other compounds having different atomic connectivity.

As used within the context of this application, the term “manmade” means engineered or purposefully created by a human in contrast to naturally “in nature” without human influence. In one embodiment, manmade compositions are formulations made by combining naturally occurring molecules in a new unnatural way, for example by separating the individual chemical components of a plant and then recombining some of those components to achieve different concentrations and/or ratios of those components. In one embodiment, manmade compositions are formulations made with one or more processes in a chemical extraction and purification lab. The properties of compositions of this disclosure—made by purposefully combining purified compounds with concentrations, combinations and/or ratios—differ from previously existing technology, such as extracts or other processed materials, e.g., biological drug substance, medically grade plant extracts, etc.

Cannabinoids and other plant molecules may be extracted using various solvents and technologies including, but not limited to ethanol, butane, methane, carbon dioxide, ice, water, steam. Cannabinoids and other plant molecules may be extracted from plants bred to express desired cannabinoid and/or terpene and/or flavonoid profiles for purity. Cannabinoids and other molecules may be purified using supercritical fluid (“SFC”) extraction and similar technologies. In one example, the process of crystallization involves placing the compound of interest in a liquid and then cooling or adding participants to the solution which would lower the solubility of the compound of interest so that it forms crystals. In this example, crystals are then separated from the liquid through filtration or centrifuge.

In one embodiment, purification comprises using various solvents, e.g., ethanol, butane, methane, carbon dioxide, ice, water, steam. In one embodiment, purification comprises various techniques, e.g., chromatography, crystallization, filtration, centrifuge, etc. or various combinations of said techniques. In one embodiment, purification comprises extracting cannabinoids and other plant molecules from plants bred to express desired cannabinoid and/or terpene profiles for purity.

As used herein, the term “cellulose” means an organic compound with the formula (C₆H₁₀O₅)ₙ. In one embodiment, the phrase “substantially free from cellulose” is intended to distinguish purified components of naturally occurring plants (for example the cannabis plant) from the isolated chemical compounds that are free from plant material, such as cellulose.

As used herein, the term “naturally occurring” refers to materializing, arising, happening, or synthesizing in nature. In one embodiment, naturally occurring refers to a secondary compound synthesized within a plant. In one embodiment, naturally occurring refers to a collection of cannabinoids synthesized within a plant of genus cannabis. In one embodiment, naturally occurring refers to the concentrations of compounds within a plant. In one embodiment, naturally occurring refers to the concentration of cannabinoids within a plant of genus cannabis. In one embodiment, naturally occurring refers to the ratio of secondary compounds within a plant. In one embodiment, the naturally occurring refers to the ratio of a first cannabinoid to a second cannabinoid within a plant of genus cannabis.

As used herein, the term “naturally occurring ratio” refers to the proportion of one compound or compounds in relation to another compound or compounds within a plant of genus cannabis. In one embodiment, the naturally occurring ratio is the amount of a cannabinoid in relation to the amount of a cannabinoid within a plant of genus cannabis. In one embodiment, the naturally occurring ratio is the amount of a cannabinoid in relation to the amount of a terpene within a plant of genus cannabis. In one embodiment, the naturally occurring ratio is the amount of a cannabinoid in relation to the amount of a terpene within a flower of genus cannabis. In one embodiment, the naturally occurring ratio is the amount of a cannabinoid in relation to the amount of a terpene within a plant extract of genus. In one embodiment, the naturally occurring ratio is the amount of a cannabinoid in relation to the amount of a terpene within a formulation made from plant extract of genus cannabis.

In one embodiment, the naturally occurring ratio is expressed as a molar ratio. In one embodiment, the naturally occurring ratio is expressed as a mass. In one embodiment, the mass and/or molar ratio is measured by chromatography and/or spectroscopy.

As used herein, the term “unnaturally occurring ratio” refers to the proportion of one compound or compounds in relation to another compound or compounds in a composition created by a human. In one embodiment, the unnaturally occurring ratio is the amount of a cannabinoid in relation to a terpene and is not observed in a plant of genus cannabis. In one embodiment, the unnaturally occurring ratio is expressed as a molar ratio. In one embodiment, the unnaturally occurring ratio is expressed as a mass. In one embodiment, the mass and/or molar ratio is measured by chromatography and/or spectroscopy.

In one embodiment, the unnaturally occurring ratio is the amount of a cannabinoid in relation to the amount of a terpene within a composition formulated by a human not observed in a plant of genus cannabis. In one embodiment, the unnaturally occurring ratio is the amount of a cannabinoid in relation to the amount of a second cannabinoid within a composition formulated by a human not observed in a plant of genus cannabis.

As used herein, the term “naturally occurring concentration” refers to the amount of a compound or compounds in relation to an entire naturally occurring reference sample. In one embodiment, the naturally occurring concentration is the amount of a cannabinoid in a sample of a plant of genus cannabis. In one embodiment, the naturally occurring concentration is the amount of a cannabinoid within the dried, or cured, flower of a plant of genus cannabis. In one embodiment, the naturally occurring concentration is the amount of a cannabinoid within a crude extract of a plant of genus cannabis.

In one embodiment, the naturally occurring concentration is the amount of a terpene in a sample of a plant of genus cannabis. In one embodiment, the naturally occurring concentration is the amount of a terpene within the dried, or cured, flower of a plant of genus cannabis. In one embodiment, the naturally occurring concentration is the amount of a terpene within a crude extract of a plant of genus cannabis. In one embodiment, the unnaturally occurring concentration is measured by moles and expressed as Molarity. In one embodiment, the naturally occurring concentration is measured by percent mass.

As used herein, the term “unnaturally occurring concentration” refers to the amount of a compound or compounds in relation to an entire sample within a manmade composition. In one embodiment, the unnaturally occurring concentration is the amount of a cannabinoid in relation to the total composition. In one embodiment, the unnaturally occurring concentration is the amount of a terpene in relation to the total composition. In one embodiment, the unnaturally occurring concentration is measured by moles and expressed as Molarity. In one embodiment, the unnaturally occurring concentration is measured by percent mass.

As used herein, the term “total mass” refers to the entire amount of matter for a given reference sample. In one embodiment, the total mass is measured by molecular mass.

The term “pharmacological fingerprint” refers to a collection of data about the activity of a single cannabinoid or terpene, or any combination of cannabinoids and/or terpenes, at one or more targets in the human body, e.g., a cannabinoid receptor, e.g., CB1, CB2, GPR55, 5HT-1A, 5HT-2A, TRPV1, serotonin receptor, or μ-opioid receptor. The compositions disclosed herein were created using cellular (e.g., pharmacological fingerprint) and/or clinical data to select and combine particular combinations of compounds having new properties.

As used herein, the term “potency” refers to the power, influence, activity, or effectiveness of the compositions disclosed herein at a particular cellular receptor. In one embodiment, potency is measured by the response of a subject. In one embodiment, potency is measured by the cellular reactivity of a cannabinoid at a receptor, e.g., CB1, CB2, GPR55, 5HT-1A, 5HT-2A, TRPV1, serotonin receptor, or μ-opioid receptor. In one embodiment, potency refers to the EC50. In one embodiment, potency refers to amplitude of response.

As used herein, the term “agonism” refers to the effect of a compound, agonist, activating a receptor and inducing a response. In one embodiment, the receptor is chosen from CB1, CB2, GPR55, 5HT-1A, 5HT-2A, TRPV1, serotonin receptor, or μ-opioid receptor. In one embodiment, the agonist is a cannabinoid. In one embodiment, the agonist is a terpene.

As used herein, the term “antagonism” refers to two or more compounds in combination having an effect. In one embodiment, antagonism is a composition comprising a cannabinoid affecting another cannabinoid, i.e., a cannabinoid affecting the binding of another cannabinoid to a receptor. In one embodiment, antagonism is a composition comprising a terpene affecting another cannabinoid, i.e., a terpene affecting the binding of another cannabinoid to a receptor.

For example, formulations of this disclosure have been achieved by expressing the human components of the endocannabinoid system (or other known targets of cannabis pharmacology) in cultured cells and treating the cells with serial dilutions of individual compounds or combinations of compounds in order to determine the EC50s of each component at each target individually, and whether adding other components shifts either the amplitude of the response or the effective concentration (EC50) of the response.

As used herein, the term “EC50” means “half maximal effective concentration,” which refers to the concentration of a formulation (e.g., a cannabinoid, terpene, or combination thereof) inducing a response halfway between the baseline and maximum after a specified exposure time. In one embodiment, the EC50 value is used as a measure of a drug's potency.

The EC50 of a graded dose response curve represents the concentration of a compound where 50% of its maximal effect is observed. The EC50 of a quantal dose response curve represents the concentration of a compound where 50% of the population exhibit a response, after a specified exposure duration.

As used herein, the term “amplitude of response” refers to the magnitude of reactivity to the compositions disclosed herein. In one embodiment, the amplitude of response is the reaction of a receptor, e.g., CB1, CB2, GPR55, 5HT-1A, 5HT-2A, TRPV1, serotonin receptor, or μ-opioid receptor, to a sample administered thereto.

For example, in one system, D9-THC activity at cannabinoid receptor 1 (CB1) has a consistent EC50 of around 1 micromolar, and a 40-50% amplitude of response compared to a known, synthetic full agonist (CP-55,940). The lower response of CB1 to THC relative to a full agonist is why THC is referred to as a “partial agonist” of the CB1 receptor.

In some embodiments, the purposefully engineered formulations disclosed herein were developed by establishing the EC50 of THC at CB1 numerous times and determining EC50s of THC in a background of constant concentrations of other cannabinoids and terpenes. This is one example of a method for determining how particular cannabis components my either synergize, add to, or compete with the THC response itself.

Using these purposefully engineered combinations (rather than naturally occurring combinations) provide a user with quantifiable control over the underlying pharmacology and biochemistry responsible for the observed physical and psychological effects. Accordingly, the compositions disclosed herein provide unprecedented control over the effects of cannabinoids and/or terpenes on the user.

In one embodiment, the composition provides a decrease in the EC50 for either or both of a first purified cannabinoid and/or a second purified cannabinoid at a particular receptor.

In one embodiment, the composition provides an increase in the EC50 for either or both of the purified cannabinoid and/or a second purified cannabinoid at a particular receptor, for example the CB1, CB2 GPR55, 5HT-1A, 5HT-2A, TRPV1, and/or μ-opioid receptor.

In one example, the compositions disclosed herein change the EC50 of THC at the CB1 receptor. In one embodiment, by using the synergistic compositions disclosed herein, the EC50 of THC is modulated, thereby providing new beneficial properties.

In one example, THC activity at the CB1 receptor has a consistent EC50 of about 1 micromolar and a 40-50% amplitude of response compared to a known, synthetic full agonist (e.g., CP-55,940).

In one example, the synergistic benefits of the compositions disclosed herein can be demonstrated by measuring the EC50 of THC at CB1 alone and then comparing it to the EC50 of THC at CB1 in the presence of other cannabinoids and terpenes.

In one example, the disclosed compositions provide a synergistic effect relative to a reference cannabinoid administered alone. In one example, the disclosed compositions add to the effects of a reference cannabinoid. In one example, the disclosed compositions compete with a reference cannabinoid at the CB1 receptor.

In one example, the disclosed compositions provide a synergistic effect relative to THC alone.

In one example, the disclosed compositions add to the effects of THC alone.

As such, the compositions disclosed herein provide for methods of increasing the response of THC at the CB1 receptor (relative to THC alone) comprising administering THC in concert with a terpene.

In one embodiment, the purified terpene is chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol.

Disclosed herein are methods of increasing the potency at a receptor, e.g. CB1, CB2 GPR55, 5HT-1A, 5HT-2A, TRPV1, serotonin receptor, μ-opioid receptor, etc., of a first purified cannabinoid in concert with a first purified terpene. In one embodiment, increasing the potency at a receptor comprises a second purified cannabinoid. In one embodiment, increasing the potency at a receptor comprises a second purified terpene.

In one embodiment, the compositions disclosed herein provide methods of increasing the response of THC at the CB1 receptor (relative to THC alone) comprising administering THC in concert with a terpene. In one example the method of increasing THC potency comprises administering THC in concert with alpha-humulene. In one example the method of increasing THC potency comprises administering THC in concert with linalool. In one example the method of increasing THC potency comprises administering THC in concert with nerolidol. In one example the method of increasing THC potency comprises administering THC in concert with limonene. In one example the method of increasing THC potency comprises administering THC in concert with alpha-terpineol. In one example the method of increasing THC potency comprises administering THC in concert with beta-pinene. In one example the method of increasing THC potency comprises administering THC in concert with p-cymene. In one example the method of increasing THC potency comprises administering THC in concert with eucalyptol.

In one embodiment, the compositions disclosed herein provide methods of increasing the response of THC at the CB1 receptor (relative to THC alone) comprising administering THC in concert with a second cannabinoid. In one example the method of increasing THC potency comprises administering THC in concert with CBC. In one example the method of increasing THC potency comprises administering THC in concert with CBG. In one example the method of increasing THC potency comprises administering THC in concert with THCV. In one example the method of increasing THC potency comprises administering THC in concert with CBD.

In one example, the disclosed compositions compete with THC at the CB1 receptor.

In one example, the EC50 of THC at CB1 is decreased (relative to THC alone) by formulating a composition comprising purified THC and a purified terpene chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol.

In one example, the EC50 of THC at CB1 is increased (relative to THC alone) by formulating a composition comprising purified THC and beta-caryophyllene.

In one embodiment of the compositions disclosed herein, the phrase “comprising a purified THC and a purified terpene” means that the composition includes between about 0.5% to about 10% combined terpene content.

An exemplary non-limiting terpene is linalool. One embodiment of this disclosure comprises 0.5-1% purified linalool. One embodiment of this disclosure comprises 1-2% purified linalool. One embodiment of this disclosure comprises 2-5% purified linalool. One embodiment of this disclosure comprises 5-10% purified linalool.

In one example, the EC50 of THC at CB1 is decreased by formulating a composition comprising purified THC and purified Limonene.

In one example, the EC50 of THC at CB1 is decreased by formulating a composition comprising purified THC and purified Nerolidol.

In one example, the EC50 of THC at CB1 is decreased by formulating a composition comprising purified THC and purified Beta-Myrcene.

In one example, the EC50 of THC at CB1 is decreased by formulating a composition comprising purified THC and purified Linalool.

In one example, the EC50 of THC at CB1 is decreased by formulating a composition comprising purified THC and purified Alpha-Caryophyllene.

In one example, the EC50 of THC at CB1 is decreased by formulating a composition comprising purified THC and purified Beta-Caryophyllene.

In one example, the EC50 of THC at CB1 is decreased by formulating a composition comprising purified THC and purified Alpha-Pinene.

In one example, the EC50 of THC at CB1 is decreased by formulating a composition comprising purified THC and purified Beta-Pinene.

In one example, the EC50 of THC at CB1 is decreased by formulating a composition comprising purified THC and purified Alpha-Bisabolol.

In one example, the EC50 of THC at CB1 is decreased by formulating a composition comprising purified THC and purified Delta-3-Carene.

In one example, the EC50 of THC at CB1 is decreased by formulating a composition comprising purified THC and purified Borneol.

In one example, the EC50 of THC at CB1 is decreased by formulating a composition comprising purified THC and purified Eucalyptol.

In one example, the EC50 of THC at CB1 is decreased by formulating a composition comprising purified THC and purified Alpha-Terpineol.

In one example, the EC50 of THC at CB1 is decreased by formulating a composition comprising purified THC and purified p-Cymene.

In one example, the EC50 of THC at CB1 is decreased by formulating a composition comprising purified THC and purified Alpha-Humulene.

In one example, the EC50 of THC at CB1 is decreased by formulating a composition comprising purified THC and purified Terpinolene.

In one example, the EC50 of THC at CB1 is decreased by formulating a composition comprising purified THC and purified Pulegone.

In one example, the EC50 of THC at CB1 is decreased by formulating a composition comprising purified THC and purified Camphene.

In one example, the EC50 of THC at CB1 is decreased by formulating a composition comprising purified THC and purified Geraniol.

In one example, the disclosed compositions increase the EC50 of a first cannabinoid at CB1 while increasing the EC50 of a second cannabinoid at CB2.

In one example, the disclosed compositions increase the EC50 of a first cannabinoid at CB1 while increasing the EC50 of a second cannabinoid at GPR55.

In one example, the disclosed compositions increase the EC50 of a first cannabinoid at CB1 while increasing the EC50 of a second cannabinoid at 5HT-1A.

In one example, the disclosed compositions increase the EC50 of a first cannabinoid at CB1 while increasing the EC50 of a second cannabinoid at 5HT-2A.

In one example, the disclosed compositions increase the EC50 of a first cannabinoid at CB1 while decreasing the EC50 of a second cannabinoid at CB2.

In one example, the disclosed compositions increase the EC50 of a first cannabinoid at CB1 while decreasing the EC50 of a second cannabinoid at GPR55.

In one example, the disclosed compositions increase the EC50 of a first cannabinoid at CB1 while decreasing the EC50 of a second cannabinoid at 5HT-1A.

In one example, the disclosed compositions increase the EC50 of a first cannabinoid at CB1 while decreasing the EC50 of a second cannabinoid at 5HT-2A.

In one example, the disclosed compositions increase the EC50 of a first cannabinoid at CB2 while increasing the EC50 of a second cannabinoid at CB1.

In one example, the disclosed compositions increase the EC50 of a first cannabinoid at CB2 while increasing the EC50 of a second cannabinoid at GPR55.

In one example, the disclosed compositions increase the EC50 of a first cannabinoid at CB2 while increasing the EC50 of a second cannabinoid at 5HT-1A.

In one example, the disclosed compositions increase the EC50 of a first cannabinoid at CB2 while increasing the EC50 of a second cannabinoid at 5HT-2A.

In one example, the disclosed compositions increase the EC50 of a first cannabinoid at CB2 while decreasing the EC50 of a second cannabinoid at CB1.

In one example, the disclosed compositions increase the EC50 of a first cannabinoid at CB2 while decreasing the EC50 of a second cannabinoid at GPR55.

In one example, the disclosed compositions increase the EC50 of a first cannabinoid at CB2 while decreasing the EC50 of a second cannabinoid at 5HT-1A.

In one example, the disclosed compositions increase the EC50 of a first cannabinoid at CB2 while decreasing the EC50 of a second cannabinoid at 5HT-2A.

In one example, the disclosed compositions increase the EC50 of a first cannabinoid at GPR55 while increasing the EC50 of a second cannabinoid at CB1.

In one example, the disclosed compositions increase the EC50 of a first cannabinoid at GPR55 while increasing the EC50 of a second cannabinoid at CB2.

In one example, the disclosed compositions increase the EC50 of a first cannabinoid at GPR55 while increasing the EC50 of a second cannabinoid at 5HT-1A.

In one example, the disclosed compositions increase the EC50 of a first cannabinoid at GPR55 while increasing the EC50 of a second cannabinoid at 5HT-2A.

In one example, the disclosed compositions increase the EC50 of a first cannabinoid at GPR55 while decreasing the EC50 of a second cannabinoid at CB1.

In one example, the disclosed compositions increase the EC50 of a first cannabinoid at GPR55 while decreasing the EC50 of a second cannabinoid at CB2.

In one example, the disclosed compositions increase the EC50 of a first cannabinoid at GPR55 while decreasing the EC50 of a second cannabinoid at 5HT-1A.

In one example, the disclosed compositions increase the EC50 of a first cannabinoid at GPR55 while decreasing the EC50 of a second cannabinoid at 5HT-2A.

In one example, the disclosed compositions increase the EC50 of a first cannabinoid at 5HT-1A while increasing the EC50 of a second cannabinoid at CB1.

In one example, the disclosed compositions increase the EC50 of a first cannabinoid at 5HT-1A while increasing the EC50 of a second cannabinoid at CB2.

In one example, the disclosed compositions increase the EC50 of a first cannabinoid at 5HT-1A while increasing the EC50 of a second cannabinoid at GPR55.

In one example, the disclosed compositions increase the EC50 of a first cannabinoid at 5HT-1A while increasing the EC50 of a second cannabinoid at 5HT-2A.

In one example, the disclosed compositions increase the EC50 of a first cannabinoid at 5HT-1A while decreasing the EC50 of a second cannabinoid at CB1.

In one example, the disclosed compositions increase the EC50 of a first cannabinoid at 5HT-1A while decreasing the EC50 of a second cannabinoid at CB2.

In one example, the disclosed compositions increase the EC50 of a first cannabinoid at 5HT-1A while decreasing the EC50 of a second cannabinoid at GPR55.

In one example, the disclosed compositions increase the EC50 of a first cannabinoid at 5HT-1A while decreasing the EC50 of a second cannabinoid at 5HT-2A.

In one example, the disclosed compositions increase the EC50 of a first cannabinoid at 5HT-2A while increasing the EC50 of a second cannabinoid at CB1.

In one example, the disclosed compositions increase the EC50 of a first cannabinoid at 5HT-2A while increasing the EC50 of a second cannabinoid at CB2.

In one example, the disclosed compositions increase the EC50 of a first cannabinoid at 5HT-2A while increasing the EC50 of a second cannabinoid at GPR55.

In one example, the disclosed compositions increase the EC50 of a first cannabinoid at 5HT-2A while increasing the EC50 of a second cannabinoid at 5HT-1A.

In one example, the disclosed compositions increase the EC50 of a first cannabinoid at 5HT-2A while decreasing the EC50 of a second cannabinoid at CB1.

In one example, the disclosed compositions increase the EC50 of a first cannabinoid at 5HT-2A while decreasing the EC50 of a second cannabinoid at CB2.

In one example, the disclosed compositions increase the EC50 of a first cannabinoid at 5HT-2A while decreasing the EC50 of a second cannabinoid at GPR55.

In one example, the disclosed compositions increase the EC50 of a first cannabinoid at 5HT-2A while decreasing the EC50 of a second cannabinoid at 5HT-1A.

In one example, the disclosed compositions decrease the EC50 of a first cannabinoid at CB1 while increasing the EC50 of a second cannabinoid at CB2.

In one example, the disclosed compositions decrease the EC50 of a first cannabinoid at CB1 while increasing the EC50 of a second cannabinoid at GPR55.

In one example, the disclosed compositions decrease the EC50 of a first cannabinoid at CB1 while increasing the EC50 of a second cannabinoid at 5HT-1A.

In one example, the disclosed compositions decrease the EC50 of a first cannabinoid at CB1 while increasing the EC50 of a second cannabinoid at 5HT-2A.

In one example, the disclosed compositions decrease the EC50 of a first cannabinoid at CB1 while decreasing the EC50 of a second cannabinoid at CB2.

In one example, the disclosed compositions decrease the EC50 of a first cannabinoid at CB1 while decreasing the EC50 of a second cannabinoid at GPR55.

In one example, the disclosed compositions decrease the EC50 of a first cannabinoid at CB1 while decreasing the EC50 of a second cannabinoid at 5HT-1A.

In one example, the disclosed compositions decrease the EC50 of a first cannabinoid at CB1 while decreasing the EC50 of a second cannabinoid at 5HT-2A.

In one example, the disclosed compositions decrease the EC50 of a first cannabinoid at CB2 while increasing the EC50 of a second cannabinoid at CB1.

In one example, the disclosed compositions decrease the EC50 of a first cannabinoid at CB2 while increasing the EC50 of a second cannabinoid at GPR55.

In one example, the disclosed compositions decrease the EC50 of a first cannabinoid at CB2 while increasing the EC50 of a second cannabinoid at 5HT-1A.

In one example, the disclosed compositions decrease the EC50 of a first cannabinoid at CB2 while increasing the EC50 of a second cannabinoid at 5HT-2A.

In one example, the disclosed compositions decrease the EC50 of a first cannabinoid at CB2 while decreasing the EC50 of a second cannabinoid at CB1.

In one example, the disclosed compositions decrease the EC50 of a first cannabinoid at CB2 while decreasing the EC50 of a second cannabinoid at GPR55.

In one example, the disclosed compositions decrease the EC50 of a first cannabinoid at CB2 while decreasing the EC50 of a second cannabinoid at 5HT-1A.

In one example, the disclosed compositions decrease the EC50 of a first cannabinoid at CB2 while decreasing the EC50 of a second cannabinoid at 5HT-2A.

In one example, the disclosed compositions decrease the EC50 of a first cannabinoid at GPR55 while increasing the EC50 of a second cannabinoid at CB1.

In one example, the disclosed compositions decrease the EC50 of a first cannabinoid at GPR55 while increasing the EC50 of a second cannabinoid at CB2.

In one example, the disclosed compositions decrease the EC50 of a first cannabinoid at GPR55 while increasing the EC50 of a second cannabinoid at 5HT-1A.

In one example, the disclosed compositions decrease the EC50 of a first cannabinoid at GPR55 while increasing the EC50 of a second cannabinoid at 5HT-2A.

In one example, the disclosed compositions decrease the EC50 of a first cannabinoid at GPR55 while decreasing the EC50 of a second cannabinoid at CB1.

In one example, the disclosed compositions decrease the EC50 of a first cannabinoid at GPR55 while decreasing the EC50 of a second cannabinoid at CB2.

In one example, the disclosed compositions decrease the EC50 of a first cannabinoid at GPR55 while decreasing the EC50 of a second cannabinoid at 5HT-1A.

In one example, the disclosed compositions decrease the EC50 of a first cannabinoid at GPR55 while decreasing the EC50 of a second cannabinoid at 5HT-2A.

In one example, the disclosed compositions decrease the EC50 of a first cannabinoid at 5HT-1A while increasing the EC50 of a second cannabinoid at CB1.

In one example, the disclosed compositions decrease the EC50 of a first cannabinoid at 5HT-1A while increasing the EC50 of a second cannabinoid at CB2.

In one example, the disclosed compositions decrease the EC50 of a first cannabinoid at 5HT-1A while increasing the EC50 of a second cannabinoid at GPR55.

In one example, the disclosed compositions decrease the EC50 of a first cannabinoid at 5HT-1A while increasing the EC50 of a second cannabinoid at 5HT-2A.

In one example, the disclosed compositions decrease the EC50 of a first cannabinoid at 5HT-1A while decreasing the EC50 of a second cannabinoid at CB1.

In one example, the disclosed compositions decrease the EC50 of a first cannabinoid at 5HT-1A while decreasing the EC50 of a second cannabinoid at CB2.

In one example, the disclosed compositions decrease the EC50 of a first cannabinoid at 5HT-1A while decreasing the EC50 of a second cannabinoid at GPR55.

In one example, the disclosed compositions decrease the EC50 of a first cannabinoid at 5HT-1A while decreasing the EC50 of a second cannabinoid at 5HT-2A.

In one example, the disclosed compositions decrease the EC50 of a first cannabinoid at 5HT-2A while increasing the EC50 of a second cannabinoid at CB1.

In one example, the disclosed compositions decrease the EC50 of a first cannabinoid at 5HT-2A while increasing the EC50 of a second cannabinoid at CB2.

In one example, the disclosed compositions decrease the EC50 of a first cannabinoid at 5HT-2A while increasing the EC50 of a second cannabinoid at GPR55.

In one example, the disclosed compositions decrease the EC50 of a first cannabinoid at 5HT-2A while increasing the EC50 of a second cannabinoid at 5HT-1A.

In one example, the disclosed compositions decrease the EC50 of a first cannabinoid at 5HT-2A while decreasing the EC50 of a second cannabinoid at CB1.

In one example, the disclosed compositions decrease the EC50 of a first cannabinoid at 5HT-2A while decreasing the EC50 of a second cannabinoid at CB2.

In one example, the disclosed compositions decrease the EC50 of a first cannabinoid at 5HT-2A while decreasing the EC50 of a second cannabinoid at GPR55.

In one example, the disclosed compositions decrease the EC50 of a first cannabinoid at 5HT-2A while decreasing the EC50 of a second cannabinoid at 5HT-1A.

In one example, the Amplitude of Response of THC at CB1 is decreased by formulating a composition comprising purified THC and purified Limonene.

In one example, the Amplitude of Response of THC at CB1 is decreased by formulating a composition comprising purified THC and purified Nerolidol.

In one example, the Amplitude of Response of THC at CB1 is decreased by formulating a composition comprising purified THC and purified Beta-Myrcene.

In one example, the Amplitude of Response of THC at CB1 is decreased by formulating a composition comprising purified THC and purified Linalool.

In one example, the Amplitude of Response of THC at CB1 is decreased by formulating a composition comprising purified THC and purified Alpha-Caryophyllene.

In one example, the Amplitude of Response of THC at CB1 is decreased by formulating a composition comprising purified THC and purified Beta-Caryophyllene.

In one example, the Amplitude of Response of THC at CB1 is decreased by formulating a composition comprising purified THC and purified Alpha-Pinene.

In one example, the Amplitude of Response of THC at CB1 is decreased by formulating a composition comprising purified THC and purified Beta-Pinene.

In one example, the Amplitude of Response of THC at CB1 is decreased by formulating a composition comprising purified THC and purified Alpha-Bisabolol.

In one example, the Amplitude of Response of THC at CB1 is decreased by formulating a composition comprising purified THC and purified Delta-3-Carene.

In one example, the Amplitude of Response of THC at CB1 is decreased by formulating a composition comprising purified THC and purified Borneol.

In one example, the Amplitude of Response of THC at CB1 is decreased by formulating a composition comprising purified THC and purified Eucalyptol.

In one example, the Amplitude of Response of THC at CB1 is decreased by formulating a composition comprising purified THC and purified Alpha-Terpineol.

In one example, the Amplitude of Response of THC at CB1 is decreased by formulating a composition comprising purified THC and purified p-Cymene.

In one example, the Amplitude of Response of THC at CB1 is decreased by formulating a composition comprising purified THC and purified Alpha-Humulene.

In one example, the Amplitude of Response of THC at CB1 is decreased by formulating a composition comprising purified THC and purified Terpinolene.

In one example, the Amplitude of Response of THC at CB1 is decreased by formulating a composition comprising purified THC and purified Camphene.

In one example, the Amplitude of Response of THC at CB1 is decreased by formulating a composition comprising purified THC and purified Geraniol.

In one example, the Amplitude of Response of THC at CB1 is decreased by formulating a composition comprising purified THC and purified Pulegone.

In one example, the Amplitude of Response of THC at CB1 is increased by formulating a composition comprising purified THC and purified Limonene.

In one example, the Amplitude of Response of THC at CB1 is increased by formulating a composition comprising purified THC and purified Nerolidol.

In one example, the Amplitude of Response of THC at CB1 is increased by formulating a composition comprising purified THC and purified Beta-Myrcene.

In one example, the Amplitude of Response of THC at CB1 is increased by formulating a composition comprising purified THC and purified Linalool.

In one example, the Amplitude of Response of THC at CB1 is increased by formulating a composition comprising purified THC and purified Alpha-Caryophyllene.

In one example, the Amplitude of Response of THC at CB1 is increased by formulating a composition comprising purified THC and purified Beta-Caryophyllene.

In one example, the Amplitude of Response of THC at CB1 is increased by formulating a composition comprising purified THC and purified Alpha-Pinene.

In one example, the Amplitude of Response of THC at CB1 is increased by formulating a composition comprising purified THC and purified Beta-Pinene.

In one example, the Amplitude of Response of THC at CB1 is increased by formulating a composition comprising purified THC and purified Alpha-Bisabolol.

In one example, the Amplitude of Response of THC at CB1 is increased by formulating a composition comprising purified THC and purified Delta-3-Carene.

In one example, the Amplitude of Response of THC at CB1 is increased by formulating a composition comprising purified THC and purified Borneol.

In one example, the Amplitude of Response of THC at CB1 is increased by formulating a composition comprising purified THC and purified Eucalyptol.

In one example, the Amplitude of Response of THC at CB1 is increased by formulating a composition comprising purified THC and purified Alpha-Terpineol.

In one example, the Amplitude of Response of THC at CB1 is increased by formulating a composition comprising purified THC and purified p-Cymene.

In one example, the Amplitude of Response of THC at CB1 is increased by formulating a composition comprising purified THC and purified Alpha-Humulene.

In one example, the Amplitude of Response of THC at CB1 is increased by formulating a composition comprising purified THC and purified Camphene.

In one example, the Amplitude of Response of THC at CB1 is increased by formulating a composition comprising purified THC and purified Terpinolene.

In one example, the Amplitude of Response of THC at CB1 is increased by formulating a composition comprising purified THC and purified Geraniol.

In one example, the Amplitude of Response of THC at CB1 is increased by formulating a composition comprising purified THC and purified Pulegone.

In one example, the disclosed compositions increase the Amplitude of Response of a first cannabinoid at CB1 while increasing the Amplitude of Response of a second cannabinoid at CB2.

In one example, the disclosed compositions increase the Amplitude of Response of a first cannabinoid at CB1 while increasing the Amplitude of Response of a second cannabinoid at GPR55.

In one example, the disclosed compositions increase the Amplitude of Response of a first cannabinoid at CB1 while increasing the Amplitude of Response of a second cannabinoid at 5HT-1A.

In one example, the disclosed compositions increase the Amplitude of Response of a first cannabinoid at CB1 while increasing the Amplitude of Response of a second cannabinoid at 5HT-2A.

In one example, the disclosed compositions increase the Amplitude of Response of a first cannabinoid at CB1 while decreasing the Amplitude of Response of a second cannabinoid at CB2.

In one example, the disclosed compositions increase the Amplitude of Response of a first cannabinoid at CB1 while decreasing the Amplitude of Response of a second cannabinoid at GPR55.

In one example, the disclosed compositions increase the Amplitude of Response of a first cannabinoid at CB1 while decreasing the Amplitude of Response of a second cannabinoid at 5HT-1A.

In one example, the disclosed compositions increase the Amplitude of Response of a first cannabinoid at CB1 while decreasing the Amplitude of Response of a second cannabinoid at 5HT-2A.

In one example, the disclosed compositions increase the Amplitude of Response of a first cannabinoid at CB2 while increasing the Amplitude of Response of a second cannabinoid at CB1.

In one example, the disclosed compositions increase the Amplitude of Response of a first cannabinoid at CB2 while increasing the Amplitude of Response of a second cannabinoid at GPR55.

In one example, the disclosed compositions increase the Amplitude of Response of a first cannabinoid at CB2 while increasing the Amplitude of Response of a second cannabinoid at 5HT-1A.

In one example, the disclosed compositions increase the Amplitude of Response of a first cannabinoid at CB2 while increasing the Amplitude of Response of a second cannabinoid at 5HT-2A.

In one example, the disclosed compositions increase the Amplitude of Response of a first cannabinoid at CB2 while decreasing the Amplitude of Response of a second cannabinoid at CB1.

In one example, the disclosed compositions increase the Amplitude of Response of a first cannabinoid at CB2 while decreasing the Amplitude of Response of a second cannabinoid at GPR55.

In one example, the disclosed compositions increase the Amplitude of Response of a first cannabinoid at CB2 while decreasing the Amplitude of Response of a second cannabinoid at 5HT-1A.

In one example, the disclosed compositions increase the Amplitude of Response of a first cannabinoid at CB2 while decreasing the Amplitude of Response of a second cannabinoid at 5HT-2A.

In one example, the disclosed compositions increase the Amplitude of Response of a first cannabinoid at GPR55 while increasing the Amplitude of Response of a second cannabinoid at CB1.

In one example, the disclosed compositions increase the Amplitude of Response of a first cannabinoid at GPR55 while increasing the Amplitude of Response of a second cannabinoid at CB2.

In one example, the disclosed compositions increase the Amplitude of Response of a first cannabinoid at GPR55 while increasing the Amplitude of Response of a second cannabinoid at 5HT-1A.

In one example, the disclosed compositions increase the Amplitude of Response of a first cannabinoid at GPR55 while increasing the Amplitude of Response of a second cannabinoid at 5HT-2A.

In one example, the disclosed compositions increase the Amplitude of Response of a first cannabinoid at GPR55 while decreasing the Amplitude of Response of a second cannabinoid at CB1.

In one example, the disclosed compositions increase the Amplitude of Response of a first cannabinoid at GPR55 while decreasing the Amplitude of Response of a second cannabinoid at CB2.

In one example, the disclosed compositions increase the Amplitude of Response of a first cannabinoid at GPR55 while decreasing the Amplitude of Response of a second cannabinoid at 5HT-1A.

In one example, the disclosed compositions increase the Amplitude of Response of a first cannabinoid at GPR55 while decreasing the Amplitude of Response of a second cannabinoid at 5HT-2A.

In one example, the disclosed compositions increase the Amplitude of Response of a first cannabinoid at 5HT-1A while increasing the Amplitude of Response of a second cannabinoid at CB1.

In one example, the disclosed compositions increase the Amplitude of Response of a first cannabinoid at 5HT-1A while increasing the Amplitude of Response of a second cannabinoid at CB2.

In one example, the disclosed compositions increase the Amplitude of Response of a first cannabinoid at 5HT-1A while increasing the Amplitude of Response of a second cannabinoid at GPR55.

In one example, the disclosed compositions increase the Amplitude of Response of a first cannabinoid at 5HT-1A while increasing the Amplitude of Response of a second cannabinoid at 5HT-2A.

In one example, the disclosed compositions increase the Amplitude of Response of a first cannabinoid at 5HT-1A while decreasing the Amplitude of Response of a second cannabinoid at CB1.

In one example, the disclosed compositions increase the Amplitude of Response of a first cannabinoid at 5HT-1A while decreasing the Amplitude of Response of a second cannabinoid at CB2.

In one example, the disclosed compositions increase the Amplitude of Response of a first cannabinoid at 5HT-1A while decreasing the Amplitude of Response of a second cannabinoid at GPR55.

In one example, the disclosed compositions increase the Amplitude of Response of a first cannabinoid at 5HT-1A while decreasing the Amplitude of Response of a second cannabinoid at 5HT-2A.

In one example, the disclosed compositions increase the Amplitude of Response of a first cannabinoid at 5HT-2A while increasing the Amplitude of Response of a second cannabinoid at CB1.

In one example, the disclosed compositions increase the Amplitude of Response of a first cannabinoid at 5HT-2A while increasing the Amplitude of Response of a second cannabinoid at CB2.

In one example, the disclosed compositions increase the Amplitude of Response of a first cannabinoid at 5HT-2A while increasing the Amplitude of Response of a second cannabinoid at GPR55.

In one example, the disclosed compositions increase the Amplitude of Response of a first cannabinoid at 5HT-2A while increasing the Amplitude of Response of a second cannabinoid at 5HT-1A.

In one example, the disclosed compositions increase the Amplitude of Response of a first cannabinoid at 5HT-2A while decreasing the Amplitude of Response of a second cannabinoid at CB1.

In one example, the disclosed compositions increase the Amplitude of Response of a first cannabinoid at 5HT-2A while decreasing the Amplitude of Response of a second cannabinoid at CB2.

In one example, the disclosed compositions increase the Amplitude of Response of a first cannabinoid at 5HT-2A while decreasing the Amplitude of Response of a second cannabinoid at GPR55.

In one example, the disclosed compositions increase the Amplitude of Response of a first cannabinoid at 5HT-2A while decreasing the Amplitude of Response of a second cannabinoid at 5HT-1A.

In one example, the disclosed compositions decrease the Amplitude of Response of a first cannabinoid at CB1 while increasing the Amplitude of Response of a second cannabinoid at CB2.

In one example, the disclosed compositions decrease the Amplitude of Response of a first cannabinoid at CB1 while increasing the Amplitude of Response of a second cannabinoid at GPR55.

In one example, the disclosed compositions decrease the Amplitude of Response of a first cannabinoid at CB1 while increasing the Amplitude of Response of a second cannabinoid at 5HT-1A.

In one example, the disclosed compositions decrease the Amplitude of Response of a first cannabinoid at CB1 while increasing the Amplitude of Response of a second cannabinoid at 5HT-2A.

In one example, the disclosed compositions decrease the Amplitude of Response of a first cannabinoid at CB1 while decreasing the Amplitude of Response of a second cannabinoid at CB2.

In one example, the disclosed compositions decrease the Amplitude of Response of a first cannabinoid at CB1 while decreasing the Amplitude of Response of a second cannabinoid at GPR55.

In one example, the disclosed compositions decrease the Amplitude of Response of a first cannabinoid at CB1 while decreasing the Amplitude of Response of a second cannabinoid at 5HT-1A.

In one example, the disclosed compositions decrease the Amplitude of Response of a first cannabinoid at CB1 while decreasing the Amplitude of Response of a second cannabinoid at 5HT-2A.

In one example, the disclosed compositions decrease the Amplitude of Response of a first cannabinoid at CB2 while increasing the Amplitude of Response of a second cannabinoid at CB1.

In one example, the disclosed compositions decrease the Amplitude of Response of a first cannabinoid at CB2 while increasing the Amplitude of Response of a second cannabinoid at GPR55.

In one example, the disclosed compositions decrease the Amplitude of Response of a first cannabinoid at CB2 while increasing the Amplitude of Response of a second cannabinoid at 5HT-1A.

In one example, the disclosed compositions decrease the Amplitude of Response of a first cannabinoid at CB2 while increasing the Amplitude of Response of a second cannabinoid at 5HT-2A.

In one example, the disclosed compositions decrease the Amplitude of Response of a first cannabinoid at CB2 while decreasing the Amplitude of Response of a second cannabinoid at CB1.

In one example, the disclosed compositions decrease the Amplitude of Response of a first cannabinoid at CB2 while decreasing the Amplitude of Response of a second cannabinoid at GPR55.

In one example, the disclosed compositions decrease the Amplitude of Response of a first cannabinoid at CB2 while decreasing the Amplitude of Response of a second cannabinoid at 5HT-1A.

In one example, the disclosed compositions decrease the Amplitude of Response of a first cannabinoid at CB2 while decreasing the Amplitude of Response of a second cannabinoid at 5HT-2A.

In one example, the disclosed compositions decrease the Amplitude of Response of a first cannabinoid at GPR55 while increasing the Amplitude of Response of a second cannabinoid at CB1.

In one example, the disclosed compositions decrease the Amplitude of Response of a first cannabinoid at GPR55 while increasing the Amplitude of Response of a second cannabinoid at CB2.

In one example, the disclosed compositions decrease the Amplitude of Response of a first cannabinoid at GPR55 while increasing the Amplitude of Response of a second cannabinoid at 5HT-1A.

In one example, the disclosed compositions decrease the Amplitude of Response of a first cannabinoid at GPR55 while increasing the Amplitude of Response of a second cannabinoid at 5HT-2A.

In one example, the disclosed compositions decrease the Amplitude of Response of a first cannabinoid at GPR55 while decreasing the Amplitude of Response of a second cannabinoid at CB1.

In one example, the disclosed compositions decrease the Amplitude of Response of a first cannabinoid at GPR55 while decreasing the Amplitude of Response of a second cannabinoid at CB2.

In one example, the disclosed compositions decrease the Amplitude of Response of a first cannabinoid at GPR55 while decreasing the Amplitude of Response of a second cannabinoid at 5HT-1A.

In one example, the disclosed compositions decrease the Amplitude of Response of a first cannabinoid at GPR55 while decreasing the Amplitude of Response of a second cannabinoid at 5HT-2A.

In one example, the disclosed compositions decrease the Amplitude of Response of a first cannabinoid at 5HT-1A while increasing the Amplitude of Response of a second cannabinoid at CB1.

In one example, the disclosed compositions decrease the Amplitude of Response of a first cannabinoid at 5HT-1A while increasing the Amplitude of Response of a second cannabinoid at CB2.

In one example, the disclosed compositions decrease the Amplitude of Response of a first cannabinoid at 5HT-1A while increasing the Amplitude of Response of a second cannabinoid at GPR55.

In one example, the disclosed compositions decrease the Amplitude of Response of a first cannabinoid at 5HT-1A while increasing the Amplitude of Response of a second cannabinoid at 5HT-2A.

In one example, the disclosed compositions decrease the Amplitude of Response of a first cannabinoid at 5HT-1A while decreasing the Amplitude of Response of a second cannabinoid at CB1.

In one example, the disclosed compositions decrease the Amplitude of Response of a first cannabinoid at 5HT-1A while decreasing the Amplitude of Response of a second cannabinoid at CB2.

In one example, the disclosed compositions decrease the Amplitude of Response of a first cannabinoid at 5HT-1A while decreasing the Amplitude of Response of a second cannabinoid at GPR55.

In one example, the disclosed compositions decrease the Amplitude of Response of a first cannabinoid at 5HT-1A while decreasing the Amplitude of Response of a second cannabinoid at 5HT-2A.

In one example, the disclosed compositions decrease the Amplitude of Response of a first cannabinoid at 5HT-2A while increasing the Amplitude of Response of a second cannabinoid at CB1.

In one example, the disclosed compositions decrease the Amplitude of Response of a first cannabinoid at 5HT-2A while increasing the Amplitude of Response of a second cannabinoid at CB2.

In one example, the disclosed compositions decrease the Amplitude of Response of a first cannabinoid at 5HT-2A while increasing the Amplitude of Response of a second cannabinoid at GPR55.

In one example, the disclosed compositions decrease the Amplitude of Response of a first cannabinoid at 5HT-2A while increasing the Amplitude of Response of a second cannabinoid at 5HT-1A.

In one example, the disclosed compositions decrease the Amplitude of Response of a first cannabinoid at 5HT-2A while decreasing the Amplitude of Response of a second cannabinoid at CB1.

In one example, the disclosed compositions decrease the Amplitude of Response of a first cannabinoid at 5HT-2A while decreasing the Amplitude of Response of a second cannabinoid at CB2.

In one example, the disclosed compositions decrease the Amplitude of Response of a first cannabinoid at 5HT-2A while decreasing the Amplitude of Response of a second cannabinoid at GPR55.

In one example, the disclosed compositions decrease the Amplitude of Response of a first cannabinoid at 5HT-2A while decreasing the Amplitude of Response of a second cannabinoid at 5HT-1A.

In one illustration of the above embodiments, the EC50 of THC at CB1 is decreased within a composition comprising THC and Alpha-Humulene.

In one embodiment, the EC50 of THC at CB1 is decreased to between about 0.1 micromolar to about 0.5 micromolar.

In one illustration of the above embodiments, the EC50 of THC at CB1 is decreased within a composition comprising THC and Linalool.

In one embodiment, the EC50 of THC at CB1 is decreased to between about 0.05 micromolar to about 0.2 micromolar.

The above described examples of cellular effect can be further correlated with the results from the clinical studies, such as those exemplified in this application.

As used herein, the term “cannabinoid” refers to a compound belonging to a class of secondary compounds commonly found in plants of genus cannabis. In one embodiment, the cannabinoid is found in a plant, e.g., a plant of genus cannabis, and is sometimes referred to as a phytocannabinoid. In one embodiment, the cannabinoid is found in a mammal, sometimes called a endocannabinoid. In one embodiment, the cannabinoid is made in a laboratory setting, sometimes called a synthetic cannabinoid. In one embodiment, the cannabinoid acts upon a cellular receptor, such as a G-coupled protein receptor (e.g., a serotonin receptor, a cannabinoid receptor, TRPV1, an opioid receptor, etc.) thereby causing a response on the brain or body. In one embodiment, the cannabinoid affects the activity of other compounds at one or more receptors by acting as an agonist, partial agonist, inverse agonist, antagonist, etc.

In many cases, a cannabinoid can be identified because its chemical name will include the text string “*cannabi* in the name.

Within the context of this application, where reference is made to a particular cannabinoid, each of the acid and/or decarboxylated forms are contemplated as both single molecules and mixtures.

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C₄ (CBD-C₄), Cannabidivarinic Acid (CBDVA), Cannabidivarin (CBDV), Cannabidiorcol (CBD-C₁), Tetrahydrocannabinolic acid A (THCA-A), Tetrahydrocannabinolic acid B (THCA-B), Tetrahydrocannabinolic Acid (THCA), Tetrahydrocannabinol (THC), Tetrahydrocannabinolic acid C₄ (THCA-C₄), Tetrahydrocannbinol C₄ (THC-C₄), Tetrahydrocannabivarinic acid (THCVA), Tetrahydrocannabivarin (THCV), Tetrahydrocannabiorcolic acid (THCA-C₁), Tetrahydrocannabiorcol (THC-C₁), Δ⁷-cis-iso-tetrahydrocannabivarin, Δ⁸-tetrahydrocannabinolic acid (A8-THCA), Cannabivarinodiolic (CBNDVA), Cannabivarinodiol (CBNDV), Δ⁸-tetrahydrocannabinol (Δ⁸-THC), Δ⁹-tetrahydrocannabinol (Δ⁹-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabivarinselsoin (CBEV), Cannabivarinselsoinic Acid (CBEVA), Cannabielsoic Acid (CBEA), Cannabielvarinsoin (CBLV), Cannabielvarinsoinic Acid (CBLVA), Cannabinolic acid (CBNA), Cannabinol (CBN), Cannabivarinic Acid (CBNVA), Cannabinol methylether (CBNM), Cannabinol-C₄ (CBN-C₄), Cannabivarin (CBV), Cannabino-C₂ (CBN-C₂), Cannabiorcol (CBN-C₁), Cannabinodiol (CBND), Cannabinodiolic Acid (CBNDA), Cannabinodivarin (CBDV), Cannabitriol (CBT), 10-Ethoxy-9-hydroxy-Δ^{6a}-tetrahydrocannabinol, 8,9-Dihydroxy-Δ^{6a(10a)}-tetrahydrocannabinol (8,9-Di-OH-CBT-05), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ^{6a(10a)}-tetrahydrocannabinol (OTHC), Δ⁹-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E,4E,8Z,10Z-tetraenoic acid isobutylamide, and Dodeca-2E,4E-dienoic acid isobutylamide.

In one embodiment, the purified cannabinoid is chosen from THC, D9-THC, D8-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

As used herein, the term “THC” refers to tetrahydrocannabinol and has the

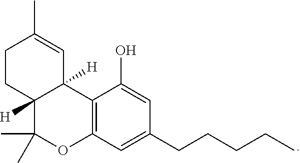 [SMILES: CCCCCc1cc(O)c2c(c1)OC(C)(C)[C@@H]1CCC(C)=C[C@@H]21]

Within the context of this disclosure, compositions comprising THC are formulated with other compounds, thereby providing previously unavailable potency, control, consistency, purity, etc.

As used herein, the term “THCA” refers to tetrahydrocannabinolic acid and has the following structural formula:

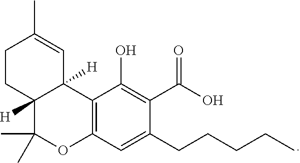 [SMILES: CCCCCc1cc2c(c(O)c1C(=O)O)[C@@H]1C=C(C)CC[C@H]1C(C)(C)O2]

Decarboxylating THCA with heat, light, etc., forms THC, D8-THC, D9-THC, and other potential cannabinoids. Within the context of this disclosure, compositions comprising THCA are formulated with other compounds, thereby providing previously unavailable potency, control, consistency, purity, etc.

As used herein, the term “THCV” refers to tetrahydrocannabivarin and has the following structural formula:

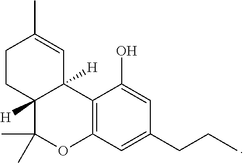 [SMILES: CCCc1cc(O)c2c(c1)OC(C)(C)[C@@H]1CCC(C)=C[C@@H]21]

Within the context of this disclosure, compositions comprising THCV are formulated with other compounds, thereby providing previously unavailable potency, control, consistency, purity, etc.

As used herein, the term “THCVA” refers to tetrahydrocannabivarinic acid and has the following structural formula:

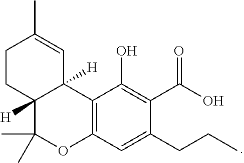 [SMILES: CCCc1cc2c(c(O)c1C(=O)O)[C@@H]1C=C(C)CC[C@H]1C(C)(C)O2]

Decarboxylating THCVA with heat, light, etc., forms THCV, D8-THCV, D9-THCV, and other possible cannabinoid derivatives. Within the context of this disclosure, compositions comprising THCVA are formulated with other compounds, thereby providing previously unavailable potency, control, consistency, purity, etc.

As used herein, the term “D8-THC” refers to delta-8-tetrahydrocannabinol and has the following structural formula:

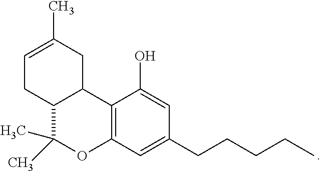

Within the context of this disclosure, compositions comprising D8-THC are formulated with other compounds, thereby providing previously unavailable potency, control, consistency, purity, etc.

As used herein, the term “D8-THCV” refers to delta-8-tetrahydrocannabivarin and has the following structural formula:

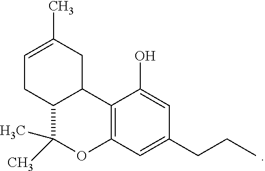

Within the context of this disclosure, compositions comprising D8-THCV are formulated with other compounds, thereby providing previously unavailable potency, control, consistency, purity, etc.

As used herein, the term “D9-THC” refers to delta-9-tetrahydrocannabinol and has the following structural formula:

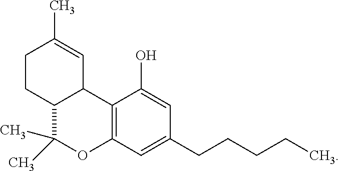

Within the context of this disclosure, compositions comprising D9-THC are formulated with other compounds, thereby providing previously unavailable potency, control, consistency, purity, etc.

As used herein, the term “D9-THCV” refers to delta-9-tetrahydrocannabivarin and has the following structural formula:

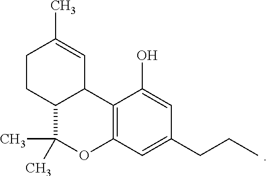

Within the context of this disclosure, compositions comprising D9-THCV are formulated with other compounds, thereby providing previously unavailable potency, control, consistency, purity, etc.

As used herein, the term “CBD” refers to cannabidiol and has the

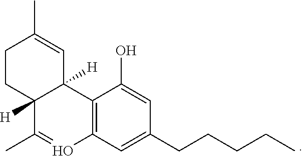

Within the context of this disclosure, compositions comprising CBD are formulated with other compounds, thereby providing previously unavailable potency, control, consistency, purity, etc.

As used herein, the term “CBDA” refers to cannabidiolic acid and has the following structural formula:

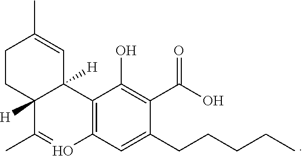

Decarboxylating CBDA with heat, light, etc., forms CBD and other possible cannabinoid derivatives. Within the context of this disclosure, compositions comprising CBDA are formulated with other compounds, thereby providing previously unavailable potency, control, consistency, purity, etc.

As used herein, the term “CBDV” refers to cannabidivarin and has the following structural formula:

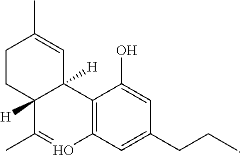

Within the context of this disclosure, compositions comprising CBDV are formulated with other compounds, thereby providing previously unavailable potency, control, consistency, purity, etc.

As used herein, the term “CBDVA” refers to cannabidivarinic acid and has the following structural formula:

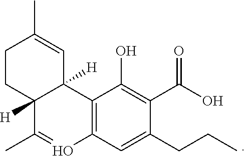

Decarboxylating CBDVA with heat, light, etc., forms CBDV and other possible cannabinoid derivatives. Within the context of this disclosure, compositions comprising CBDVA are formulated with other compounds, thereby providing previously unavailable potency, control, consistency, purity, etc.

As used herein, the term “CBC” refers to cannabichromene and has the following structural formula:

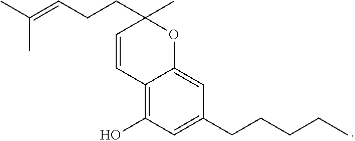 [SMILES: CCCCCc1cc(O)c2c(c1)OC(C)(CCC=C(C)C)C=C2]

Within the context of this disclosure, compositions comprising CBC are formulated with other compounds, thereby providing previously unavailable potency, control, consistency, purity, etc.

As used herein, the term “CBCA” refers to cannabichromenic acid and has the following structural formula:

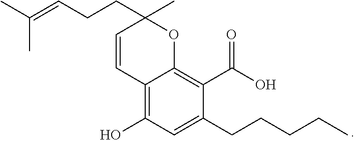 [SMILES: CCCCCc1cc(O)c2c(c1C(=O)O)OC(C)(CCC=C(C)C)C=C2]

Decarboxylating CBCA with heat, light, etc., forms CBC and other possible cannabinoid derivatives. Within the context of this disclosure, compositions comprising CBCA are formulated with other compounds, thereby providing previously unavailable potency, control, consistency, purity, etc.

As used herein, the term “CBCV” refers to cannabichromevarin and has the following structural formula:

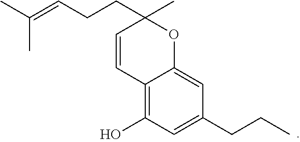 [SMILES: CCCc1cc(O)c2c(c1)OC(C)(CCC=C(C)C)C=C2]

Within the context of this disclosure, compositions comprising CBCV are formulated with other compounds, thereby providing previously unavailable potency, control, consistency, purity, etc.

As used herein, the term “CBCVA” refers to cannabichromevarinic acid and has the following structural formula:

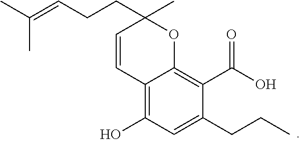 [SMILES: CCCc1cc(O)c2c(c1C(=O)O)OC(C)(CCC=C(C)C)C=C2]

Decarboxylating CBCVA with heat, light, etc., forms CBCV and other possible cannabinoid derivatives. Within the context of this disclosure, compositions comprising CBCVA are formulated with other compounds, thereby providing previously unavailable potency, control, consistency, purity, etc.

As used herein, the term “CBG” refers to cannabigerol and has the

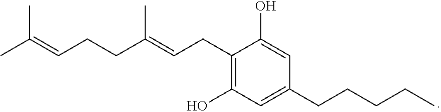 [SMILES: CCCCCc1cc(O)c(C/C=C(\C)CCC=C(C)C)c(O)c1]

Within the context of this disclosure, compositions comprising CBG are formulated with other compounds, thereby providing previously unavailable potency, control, consistency, purity, etc.

As used herein, the term “CBGA” refers to cannabigerolic acid and has the following structural formula:

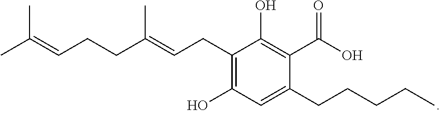 [SMILES: CCCCCc1cc(O)c(C/C=C(\C)CCC=C(C)C)c(O)c1C(=O)O]

Decarboxylating CBGA with heat, light, etc., forms CBG and other possible cannabinoid derivatives. Within the context of this disclosure, compositions comprising CBGA are formulated with other compounds, thereby providing previously unavailable potency, control, consistency, purity, etc.

As used herein, the term “CBGV” refers to cannabigerovarin and has the following structural formula:

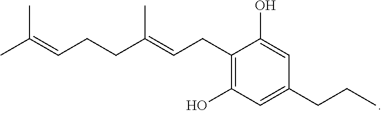 [SMILES: CCCc1cc(O)c(C/C=C(\C)CCC=C(C)C)c(O)c1]

Within the context of this disclosure, compositions comprising CBGV are formulated with other compounds, thereby providing previously unavailable potency, control, consistency, purity, etc.

As used herein, the term “CBGVA” refers to cannabigerovarinic acid and has the following structural formula:

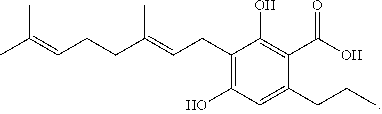 [SMILES: CCCc1cc(O)c(C/C=C(\C)CCC=C(C)C)c(O)c1C(=O)O]

Decarboxylating CBGVA with heat, light, etc., forms CBGV and other possible cannabinoid derivatives. Within the context of this disclosure, compositions comprising CBGVA are formulated with other compounds, thereby providing previously unavailable potency, control, consistency, purity, etc.

As used herein, the term “CBN” refers to cannabinol and has the following structural formula:

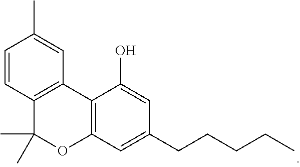 [SMILES: CCCCCc1cc(O)c2c(c1)OC(C)(C)c1ccc(C)cc1-2]

Within the context of this disclosure, compositions comprising CBN are formulated with other compounds, thereby providing previously unavailable potency, control, consistency, purity, etc.

As used herein, the term “CBNA” refers to cannabinolic acid and has the following structural formula:

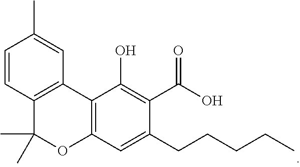 [SMILES: CCCCCc1cc2c(c(O)c1C(=O)O)-c1cc(C)ccc1C(C)(C)O2]

Decarboxylating CBNA with heat, light, etc., forms CBN and other possible cannabinoid derivatives. Within the context of this disclosure, compositions comprising CBNA are formulated with other compounds, thereby providing previously unavailable potency, control, consistency, purity, etc.

As used herein, the term “CBNV” or “CBV” refers to cannabivarin and has the following structural formula:

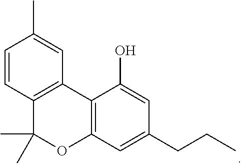 [SMILES: CCCc1cc(O)c2c(c1)OC(C)(C)c1ccc(C)cc1-2]

Within the context of this disclosure, compositions comprising CBNV are formulated with other compounds, thereby providing previously unavailable potency, control, consistency, purity, etc.

As used herein, the term “CBNVA” refers to cannabivarinic acid and has the following structural formula:

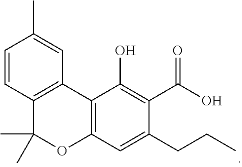 [SMILES: CCCc1cc2c(c(O)c1C(=O)O)-c1cc(C)ccc1C(C)(C)O2]

Decarboxylating CBNVA with heat, light, etc., forms CBNV and other possible cannabinoid derivatives. Within the context of this disclosure, compositions comprising CBNVA are formulated with other compounds, thereby providing previously unavailable potency, control, consistency, purity, etc.

As used herein, the term “CBND” refers to cannabinodiol and has the following structural formula:

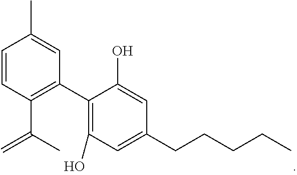 [SMILES: C=C(C)c1ccc(C)cc1-c1c(O)cc(CCCCC)cc1O]

Within the context of this disclosure, compositions comprising CBND are formulated with other compounds, thereby providing previously unavailable potency, control, consistency, purity, etc.

As used herein, the term “CBNDA” refers to cannabinodiolic acid and has the following structural formula:

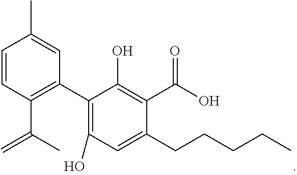 [SMILES: C=C(C)c1ccc(C)cc1-c1c(O)cc(CCCCC)c(C(=O)O)c1O]

Decarboxylating CBNDA with heat, light, etc., forms CBND and other possible cannabinoid derivatives. Within the context of this disclosure, compositions comprising CBNDA are formulated with other compounds, thereby providing previously unavailable potency, control, consistency, purity, etc.

As used herein, the term “CBNDV” refers to cannabivarinodiol and has the following structural formula:

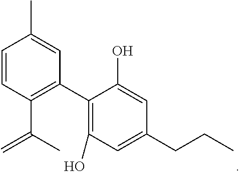 [SMILES: C=C(C)c1ccc(C)cc1-c1c(O)cc(CCC)cc1O]

Within the context of this disclosure, compositions comprising CBNDV are formulated with other compounds, thereby providing previously unavailable potency, control, consistency, purity, etc.

As used herein, the term “CBNDVA” refers to cannabivarinodiolic acid and has the following structural formula:

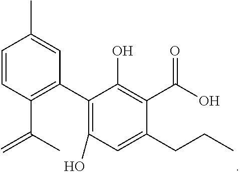 [SMILES: C=C(C)c1ccc(C)cc1-c1c(O)cc(CCC)c(C(=O)O)c1O]

Decarboxylating CBNDVA with heat, light, etc., forms CBNDV and other possible cannabinoid derivatives. Within the context of this disclosure, compositions comprising CBNDVA are formulated with other compounds, thereby providing previously unavailable potency, control, consistency, purity, etc.

As used herein, the term “CBL” refers to cannabicyclol and has the following structural formula:

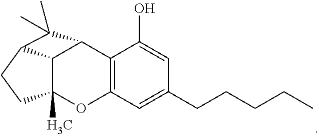

Within the context of this disclosure, compositions comprising CBL are formulated with other compounds, thereby providing previously unavailable potency, control, consistency, purity, etc.

As used herein, the term “CBLA” refers to cannabicyclolic acid and has the following structural formula:

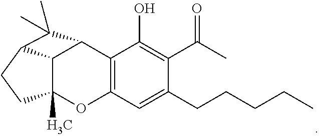

Decarboxylating CBLA with heat, light, etc., forms CBL and other possible cannabinoid derivatives. Within the context of this disclosure, compositions comprising CBLA are formulated with other compounds, thereby providing previously unavailable potency, control, consistency, purity, etc.

As used herein, the term “CBLV” refers to cannabicyclovarin and has the following structural formula:

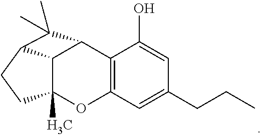

Within the context of this disclosure, compositions comprising CBLV are formulated with other compounds, thereby providing previously unavailable potency, control, consistency, purity, etc.

As used herein, the term “CBLVA” refers to cannabielvarinsoinic acid and has the following structural formula:

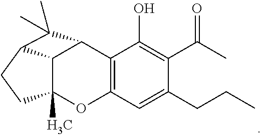

Decarboxylating CBLVA with heat, light, etc., forms CBLV and other possible cannabinoid derivatives. Within the context of this disclosure, compositions comprising CBLVA are formulated with other compounds, thereby providing previously unavailable potency, control, consistency, purity, etc.

As used herein, the term “CBE” refers to cannabielsoin and has the following structural formula:

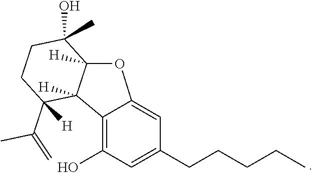

Within the context of this disclosure, compositions comprising CBE are formulated with other compounds, thereby providing previously unavailable potency, control, consistency, purity, etc.

As used herein, the term “CBEA” refers to cannabielsoic acid and has the following structural formula:

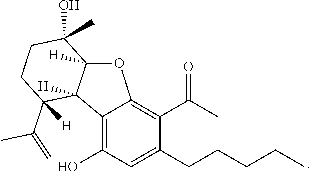

Decarboxylating CBEA with heat, light, etc., forms CBE and other possible cannabinoid derivatives. Within the context of this disclosure, compositions comprising CBEA are formulated with other compounds, thereby providing previously unavailable potency, control, consistency, purity, etc.

As used herein, the term “CBEV” refers to cannabivarinselsoin and has the following structural formula:

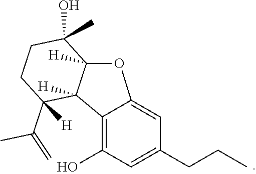

Within the context of this disclosure, compositions comprising CBEV are formulated with other compounds, thereby providing previously unavailable potency, control, consistency, purity, etc.

As used herein, the term “CBEVA” refers to cannabivarinselsoinic acid and has the following structural formula:

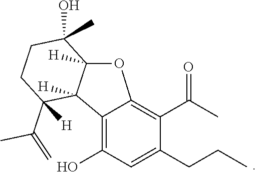

Decarboxylating CBEVA with heat, light, etc., forms CBEV and other possible cannabinoid derivatives. Within the context of this disclosure, compositions comprising CBEVA are formulated with other compounds, thereby providing previously unavailable potency, control, consistency, purity, etc.

As used herein, the term “terpene” refers to a compound built on an isoprenoid structure or produced by combining isoprene units, 5 carbon structures. Terpenes are also associated with producing smell in plants where terpenes are part of a class of secondary compounds. In one embodiment, the terpene is a hydrocarbon.

Within the context of this disclosure, the term “terpene” does not necessarily require 5 carbons or multiples of 5 carbons. It is understood that a reaction with isoprene units does not always result in a terpene comprising all the carbon atoms.

Within the context of this disclosure, the term “terpene” includes Hemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives. As well as isomeric, enantiomeric, or optically active derivatives.

Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids.

Within the context of this disclosure, the term terpene includes the α- (alpha), β- (beta), γ- (gamma), oxo-, isomers, or any combinations thereof.

Examples of terpenes within the context of this disclosure include: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Alpha-Caryophyllene, Beta-Caryophyllene, Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thuj one, Thymol, Alpha-Tocopherol, Tonka Undecanone, Undecanal, Valeraldehyde/Pentanal, Verdoxan, Alpha-Ylangene, Umbelliferone, or Vanillin.

As used herein, the term “7,8-dihydro-alpha-ionone” refers to a compound with the following structural formula:

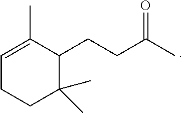

7,8-dihydro-alpha-ionone is often characterized as having a woody, floral odor.

As used herein, the term “7,8-dihydro-beta-ionone” refers to a compound with the following structural formula:

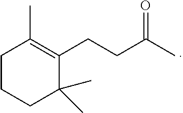

7,8-dihydro-beta-ionone is often characterized as having a woody aroma.

As used herein, the term “acetanisole” refers to a compound with the

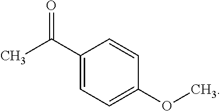

Acetanisole is often characterized as having a sweet, anisic, vanilla-like aroma with powdery, balsamic, and benzaldehyde nuances. Acetanisole is also used as a flavoring agent.

As used herein, the term “acetic acid” refers to a compound with the following structural formula:

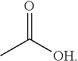

Acetic acid is often characterized as a one of the main constituents of vinegar.

As used herein, the term “acetyl cedrene” refers to a compound with the following structural formula:

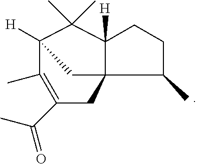 [SMILES: CC(=O)C1=C(C)[C@@H]2C[C@@]3(C1)[C@H](C)CC[C@H]3C2(C)C]

Acetyl cedrene is often characterized as having a warm, woody, amber musky aroma. Acetyl cedrene is also a fragrance agent.

As used herein, the term “anethole” refers to an aromatic compound having the following structural formula:

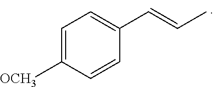

Anethole is often characterized as having a sweet, anise, licorice aroma. Anethole is used in a wide variety of fragrances and flavors.

As used herein, the term “anisole” refers to a compound with the following structural formula:

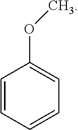

Anisole is often characterized as smelling like anise seeds.

As used herein, the term “benzaldehyde” refers to a compound with the following structural formula:

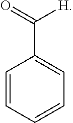

Benzaldehyde is often characterized as having an almond-like odor.

As used herein, the term “bergamotene” refers to a compound including either or both of alpha-cis-bergamotene and/or alpha-trans-Bergamotene in a pure and/or mixture of any ratio.

Alpha-cis-bergamotene refers to a compound with the following structural formula:

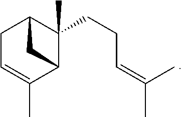

Alpha-cis-bergamotene is often characterized as having a strong odor of ground black pepper. Alpha-cis-bergamotene is an aroma component of many species of the family orchidaceae.

Alpha-trans-Bergamotene refers to the following structural formula:

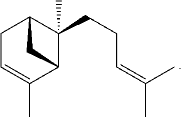

Alpha-trans-Bergamotene is often characterized as having a medium strength, warm, tea-leaf-like odor. Alpha-trans-Bergamotene is used in the chemical communication system of some species of aphids.

As used herein, the term “bisabolol” refers to a compound including either or both of alpha bisabolol and/or beta bisabolol in a pure and/or mixture of any ratio.

Alpha-bisabolol refers to a compound with the following structural formula:

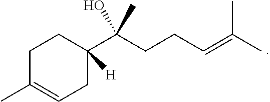 [SMILES: CC(C)=CCC[C@@](C)(O)[C@@H]1CC=C(C)CC1]

Alpha-bisabolol is often characterized as the active component of chamomile.

Beta-bisabolol refers to a compound with the following structural formula:

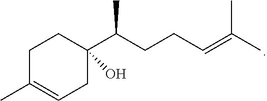 [SMILES: CC(C)=CCC[C@H](C)[C@@]1(O)CC=C(C)CC1]

Beta-bisabolol is often characterized as having a citrus, floral, lemon, sweet, herbaceous aroma.

As used herein, the term “borneol” refers to a compound having the following structural formula:

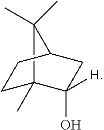

Borneol is often characterized as having a menthol aroma, similar to camphor. Borneol can also be converted into camphor.

As used herein, the term “bornyl acetate” refers to a compound with the

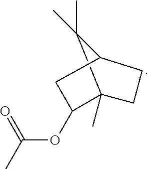

Bornyl acetate is often characterized as having a pine, camphoraceous, herbal, and balsamic odor.

As used herein, “butanoic/butyric acid” refers to a compound with the following structural formula:

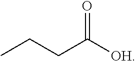

Butyric acid is often characterized as having an unpleasant, acrid odor normally found in perspiration, flatulence, and rancid butter.

As used herein, the term “cadinene” refers to a compound including either or both of alpha-cadinene and/or gamma-cadinene in a pure and/or mixture of any ratio.

Alpha-cadinene refers to a compound with the following structural formula:

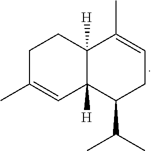

Alpha-cadinene is often characterized as having a pungent, smoky, woody, guaiac wood-like odor.

Gamma-cadinene refers to a compound having either of the following structural formula:

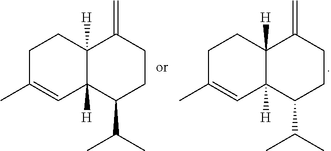

Gamma-cadinene is often characterized as having an herbaceous, herbal, woody aroma. Gamma-cadinene has shown some antimicrobial and antibacterial properties. Many species of termites and a few beetles utilize gamma-cadinene in their chemical communication systems.

As used herein, the term “cafestol” refers to a compound with the following structural formula:

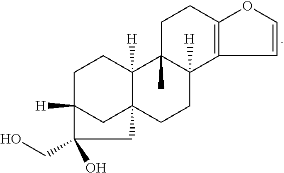

Cafestol is often characterized as major constituent in coffee.

As used herein, the term “caffeic acid” refers to a compound with the

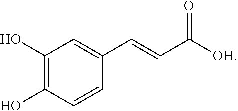 [SMILES: O=C(O)/C=C/c1ccc(O)c(O)c1]

Caffeic acid is often characterized as a key intermediate in the synthesis of lignin.

As used herein, the term “camphene” refers to a compound with the following structural formula:

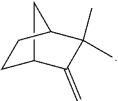

Camphene is often characterized as having a pungent, herbal, fir needle smell. Camphene is used in fragrances and food additives. Camphene is a minor constituent of many essential oils such as turpentine, cypress oil, camphor oil, citronella oil, neroli, ginger oil, and valerian.

As used herein, the term “camphor” refers to a compound with either of the following structural formulas:

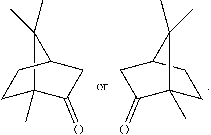

Within the context of this disclosure, the term “camphor” includes enantiomers as either a single form or a mixture in any ratio. Camphor has a very characteristic odor for which the tree is named. Camphor is used as a flavor and fragrance agent in chewing gum, hard candy, etc.

As used herein, the term “capsaicin” refers to a chemical compound with following structural formula:

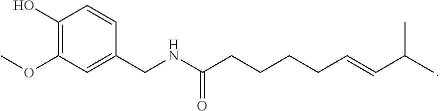 [SMILES: COc1cc(CNC(=O)CCCC/C=C/C(C)C)ccc1O]

Capsaicin is often odorless. Capsaicin is often characterized as an irritant for mammals, known for the sensation it provokes when inhaled, eaten, or applied to the skin.

As used herein, the term “carene” or “delta-3-carene” refers to a compound with the following structural formula:

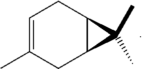

Delta-3-carene, sometimes referred to as “Carene”, is often characterized as having a sweet and pungent odor.

As used herein, the term “carotene” refers to any one of a series of related compounds having the chemical formula C₄₀Hₓ.

Within the context of this disclosure, the term “carotene” refers to any of the isomeric forms of carotene in a pure and/or mixture in any ratio. Carotene is often characterized as an important compound in the photosynthesis process.

As used herein, term “carvacrol” refers to a compound with the following structural formula:

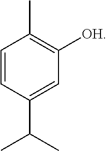

Carvacrol is often characterized as having a pungent odor of oregano. Carvacrol is often used as a flavor and fragrance agent and its flavor has been described as spicy, herbal, phenolic, medicinal, and woody.

As used herein, the term “dextro-carvone” refers to the S-(+) enantiomer of carvone with the following structural formula:

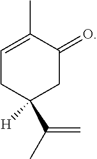

Dextro-carvone is often characterized as having a spicy, bready, caraway aroma. Dextro-carvone is found in mandarin peel oil and gingergrass oil. Dextro-carvone is the principal constituent of the oil from caraway seeds.

As used herein, the term “laevo-carvone” refers to the R-(−) enantiomer of carvone with the following structural formula:

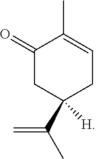

Laevo-carvone is often characterized as having a sweet, minty, herbaceous, spearmint odor. Laevo-carvone is found in spearmint and kuromoji oils.

As used herein, the term “beta-caryophyllene” refers to a compound with the following structural formula:

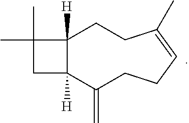

Beta-Caryophyllene is often characterized as a flavor component of black pepper. Beta-caryophyllene is often referred to as caryophyllene.

As used herein, the term “caryophyllene oxide” refers a compound with the following structural formula:

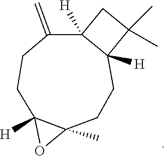 [SMILES: C=C1CC[C@H]2O[C@]2(C)CC[C@@H]2[C@@H]1CC2(C)C]

Caryophyllene oxide is often characterized as having a lemon balm odor.

As used herein, the term “cedrene” refers to either or both of alpha-cedrene and/or beta-Cedrene as pure forms or mixtures in any ratio.

Alpha-cedrene refers to a compound having the following structural formula:

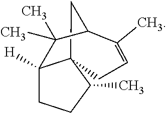

Alpha-cedrene is often characterized as having a medium strength, woody, sweet, fresh aroma of cedar. Alpha-cedrene is used in bakery items, sherbet and sorbet. Alpha-cedrene is a major component in the essential oil of cedar.

Beta-cedrene refers to a compound with the following structural formula:

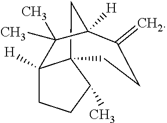

Beta-cedrene is often characterized as having a woody aroma of cedar. Beta-cedrene is a major component in the essential oil of cedar.

As used herein, the term “cedrene epoxide” or “alpha-cedrene epoxide” refers to a compound with the following structural formula:

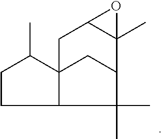 [SMILES: CC1CCC2C(C)(C)C3CC12CC1OC13C]

Alpha-cedrene epoxide is often characterized as having a woody, amber, tobacco, sandalwood, and fresh patchouli aroma. Alpha-cedrene epoxide is commonly used as a fragrance agent and a perfuming agent for cosmetics.

As used herein, the term “cedrol” refers to a compound with the following structural formula:

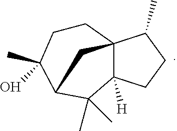 [SMILES: C[C@@H]1CC[C@H]2C(C)(C)[C@H]3C[C@@]12CC[C@@]3(C)O]

Cedrol is often characterized as having a very faint aroma that is sweet, cedar, and woody. Cedrol is a major component of cedar wood oil.

As used herein, the term “cembrene A” refers to a compound with the following structural formula:

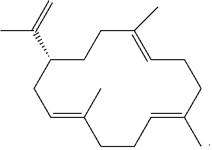 [SMILES: C=C(C)[C@H]1C/C=C(\C)CC/C=C(\C)CC/C=C(\C)CC1]

Cembrene A is often characterized as having a faint wax like odor.

As used herein, the term “chlorogenic acid” refers to a compound with the following structural formula:

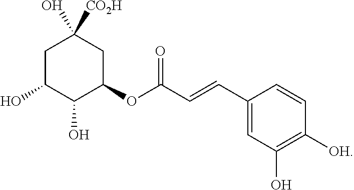 [SMILES: O=C(/C=C/c1ccc(O)c(O)c1)O[C@@H]1C[C@](O)(C(=O)O)C[C@@H](O)[C@H]1O]

Chlorogenic acid can often be found in peaches, prunes, and green coffee bean extract.

As used herein, the term “cinnamaldehyde” refers to a compound with the following structural formula:

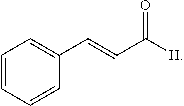

Cinnamaldehyde is often characterized as having a spicy, sweet cinnamon odor, and gives cinnamon its flavor. Cinnamaldehyde occurs naturally in the bark of cinnamon trees and other species of the genus Cinnamomum.

As used herein, the term “alpha-hexyl-cinnamaldehyde” refers to a compound with the following structural formula:

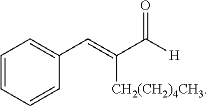

Alpha-hexyl-cinnamaldehyde is often characterized as having a sweet, floral, green, jasmine, citrus, and fruity aroma with powdery, tropical or spicy notes.

As used herein, the term “alpha-amyl-cinnamaldehyde” refers to a compound with the following structural formula:

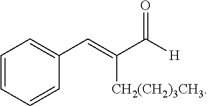

Alpha-amyl-cinnamaldehyde is often characterized as having a sweet, floral, fruity, herbal, jasmine, and tropical aroma.

As used herein, the term “cinnamic acid” refers to a compound with the following structural formula:

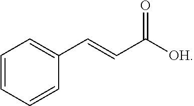 [SMILES: O=C(O)/C=C/c1ccccc1]

Cinnamic acid is often characterized as having a balsamic, sweet, storax, honey-like odor. Cinnamic acid is obtained from the oil of cinnamon or from balsams such as storax.

As used herein, the term “cinnamyl alcohol” refers to a compound with the following structural formula:

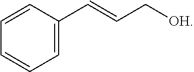

Cinnamyl alcohol is often characterized as having a cinnamon spice, floral green, and fermented odor with powdery balsamic nuances. As a flavor component cinnamyl alcohol has a green, floral, spicy, and honey flavor with a fermented yeasty nuance.

As used herein, the term “citronellal” refers to a compound with the following structural formula:

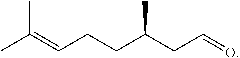

Citronellal is often characterized as making up to 80% of the leaf oil from Kaffir lime leaves and as the characteristic aroma.

As used herein, the term “citronellol” refers to either or both of the (+) and (−) enantiomers as pure forms or mixtures in any ratio.

The (+) enantiomer has the following structural formula:

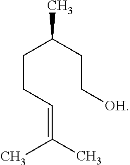

The (−) form has the following structural formula:

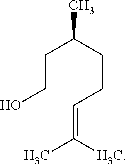

Citronellol is often characterized as having a floral, rosy, sweet, citrus with green, fatty, terpene-nuanced odor.

As used herein, the term “cryptone” refers to a compound with the following structural formula:

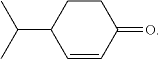

Cryptone is often characterized as having a woody minty herbaceous smell.

As used herein, the term “curcumene” refers either or both alpha-curcumene and/or gamma-curcumene as pure forms or mixtures in any ratio.

Alpha-curcumene has the following structural formula:

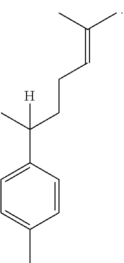

Alpha-curcumene is often characterized as having an odor similar to turmeric. Alpha-curcumene is found prominently in the Zingiber genus of ginger.

Gamma-curcumene has the following structural formula:

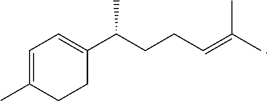

Gamma-curcumene is often characterized as having an earthy aroma. Gamma-curcumene is found prominently in the Libocedrus bidwillii tree.

As used herein, the term “decanal” refers to a compound with the following structural formula:

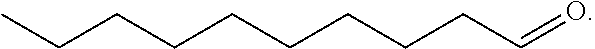

Decanal is often characterized as having a citrus odor. Decanal is used as a fragrance and flavoring agent.

As used herein, the term “dehydrovomifoliol” refers to a compound with the following structural formula:

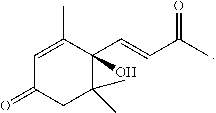 [SMILES: CC(=O)/C=C/[C@@]1(O)C(C)=CC(=O)CC1(C)C]

Dehydrovomifoliol is often characterized as having a fruity and flowery odor.

As used herein, the term “diallyl disulfide” refers to a compound with the following structural formula:

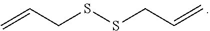

Diallyl disulfide is often characterized as having a strong, alliaceous, onion, and garlic-like odor.

As used herein, the term “dihydroactinidiolide” refers to a compound w the following structural formula:

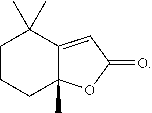

Dihydroactinidiolide is often characterized as having a fruity, musky, coumarin tea-like, peach aroma.

As used herein, the term “dimethyl disulfide” refers to a compound with the following structural formula:

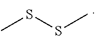

Dimethyl disulfide is often characterized as having a sulfurous, rotten aroma.

As used herein, the term “eicosane” or “icosane” refers to a compound with the chemical formula C₂₀H₄₂ and having 366,619 constitutional isomers with the following skeletal structure:

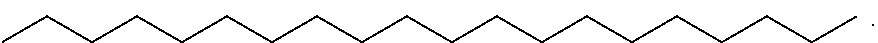 [SMILES: CCCCCCCCCCCCCCCCCCCC]

Eicosane is often characterized as having a waxy odor. Eicosane is used in fragrance concentrates.

As used herein, the term “beta-elemene” refers to a compound with the following structural formula:

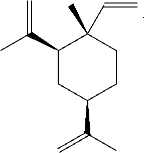

Beta-elemene is often characterized as having a sweet aroma.

As used herein, the term “estragole” refers to a compound with the

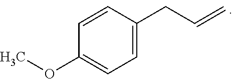

Estragole is an isomer of anethole. Estragole is often characterized as having an anise seed smell and occurs in tarragon oil, turpentine, and other essential oils.

As used herein, the term “ethyl acetate” refers to a compound with the following structural formula:

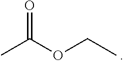

Ethyl acetate is often characterized as having an acidic, fruity, dirty, cheesy, fermented odor.

As used herein, the term “ethyl cinnamate” refers to a compound with the following structural formula:

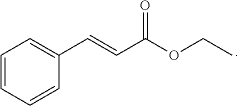

Ethyl Cinnamate is often characterized as having a sweet, balsamic, spicy, fruity, berry, and plum odor.

As used herein, the term “ethyl maltol” refers to a compound with the

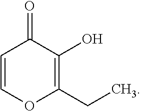

Ethyl maltol is often characterized as having a sweet smell similar to caramelized sugar and cooked fruit.

As used herein, the term “eucalyptol” refers to a terpene with the following structural formula:

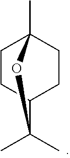

Eucalyptol is often characterized as having a mint-like smell. Eucalyptol is also known by 1,8-cineol, 1,8-cineole, cajeputol, 1,8-epoxy-p-menthane, 1,8-oxido-p-menthane, eucalyptol, eucalyptole, 1,3,3-trimethyl-2-oxabicyclo[2,2,2]octane, cineol, and cineole.

As used herein, the term “eudesmol” refers to alpha-eudesmol, beta-eudesmol, or gamma-eudesmol as pure forms or mixtures in any ratio.

Alpha-eudesmol has the following structural formula:

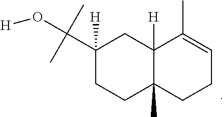 [SMILES: CC1=CCC[C@]2(C)CC[C@@H](C(C)(C)O)CC12]

Alpha-eudesmol is often characterized as having a sweet, woody odor.

Beta-eudesmol has the following structural formula:

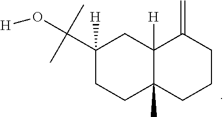 [SMILES: C=C1CCC[C@]2(C)CC[C@@H](C(C)(C)O)CC12]

Beta-eudesmol is often characterized as having a sweet, green, woody, yuzu-like aroma.

Gamma-eudesmol has the following structural formula:

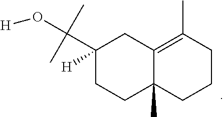 [SMILES: CC1=C2C[C@H](C(C)(C)O)CC[C@@]2(C)CCC1]

Gamma-eudesmol is often characterized as having a waxy, sweet, woody, floral odor.

As used herein the term “eugenol” refers to a compound with the following structural formula:

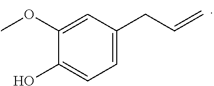

Eugenol is often characterized as causing the aromatic smell of cloves. Eugenol is found in insect attractants as well as UV absorbers.

As used herein, the term “euphol” refers to a compound with the following structural formula:

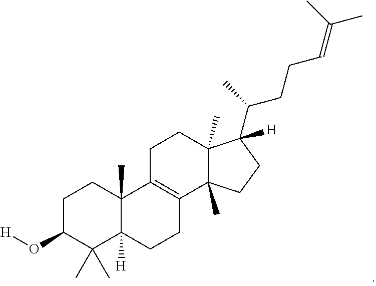 [SMILES: CC(C)=CCC[C@@H](C)[C@@H]1CC[C@]2(C)C3=C(CC[C@@]12C)[C@@]1(C)CC[C@H](O)C(C)(C)[C@@H]1CC3]

Euphol is often characterized as the main constituent in the sap of Euphorbia tirucalli.

As used herein, the term “farnesene” refers to six closely related compounds. (E,E)-alpha-farnesene is one of these six molecules and has the following structural formula:

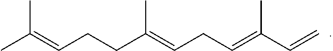

Within the context of this disclosure, the term “farnesene” refers to any one of the six closely related compounds, either alone or in combination of any other of those six closely related compounds. Farnesene is often characterized as having a fragrance of magnolia flowers and has citrus notes with green, woody, vegetative odor with hints of lavender.

As used herein, the term “farnesol” refers to a compound with the following structural formula:

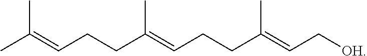 [SMILES: CC(C)=CCC/C(C)=C/CC/C(C)=C/CO]

Farnesol is often characterized as having a mild, fresh, sweet, floral, linden tree odor. Farnesol is used in cosmetics, flavors and fragrances.

As used herein, the term “fenchol” or “beta-fenchol” refers to a compound with the following structural formula:

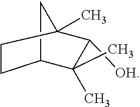

Beta-fenchol is often characterized as having a camphorous, piney, woody, dry, sweet, lemon scent. Beta-fenchol is used as a flavor and fragrance agent.

As used herein, the term “fenchone” refers to a compound with the following structural formula:

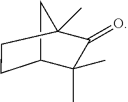

Fenchone is often characterized as having a camphorous, thuja, cedar leaf, herbal, earthy, woody aroma.

As used herein, the term “geraniol” refers to a compound of the following structural formula:

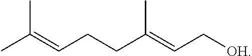

Geraniol is often characterized as having a sweet-rose like scent.

As used herein, the term “geranyl acetate” refers to a compound with the following structural formula:

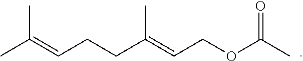

Geranyl acetate is often characterized as having a floral and fruit aroma. Geranyl acetate is found in a variety of natural oils from plants such as citronella, lemon grass, sassafras, rose, and many others.

As used herein, the term “geranylfarnesol” refers to a compound with the following structural formula:

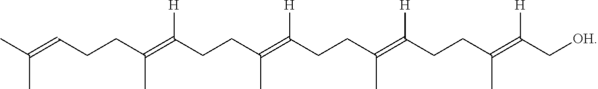 [SMILES: CC(C)=CCC/C(C)=C/CC/C(C)=C/CC/C(C)=C/CC/C(C)=C/CO]

Geranylfarnesol is often characterized as a clear colorless liquid.

As used herein, the term “germacrene” refers to a class of hydrocarbons. Within the context of this disclosure, the term “germacrene” refers to any of the five isomers as either pure forms or in any combination of the five isomers.

Germacrene A refers to the following structural formula:

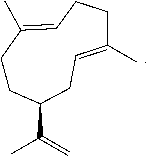

Germacrene B refers to the following structural formula:

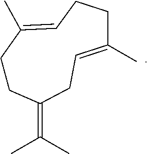

Germacrene C refers to the following structural formula:

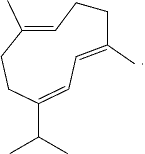

Germacrene D refers to the following structural formula:

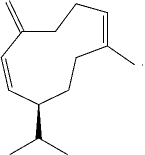

Germacrene E refers to the following structural formula:

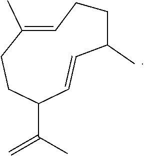

As used herein, the term “guaia-1(10), 11-diene” refers to a compound with the following structural formula:

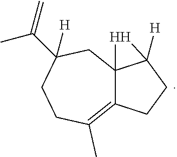

Guaia-1(10),11-diene is often characterized as having a sweet, woody aroma. Guaia-1(10),11-diene is used as a fragrance for a wide range of products from food additives, tobacco flavorings and general cosmetics, to odorizing rooms.

As used herein, the term “guaiacol” refers to a compound with the following structural formula:

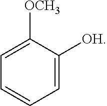

Guaiacol is often characterized as having a smoke-like, spicy, woody, medicinal odor. Guaiacol has a sweet, powdery, musty, vanilla, floral, almond flavor.

As used herein, the term “alpha-guaiene” refers to a compound with the following structural formula:

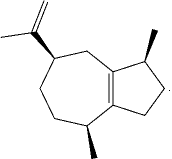

Alpha-guaiene is often characterized as having a sweet, earthy, woody, balsamic, peppery aroma.

As used herein, the term “gurjunene” or “alpha-gurjunene” refers to a compound with the following structural formula:

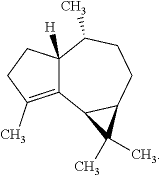

Alpha-gurjunene is often characterized as having a woody, balsamic odor. Alpha-gurjunene is used in cosmetics and fragrances.

As used herein, the term “herniarin” refers to a compound with the following structural formula:

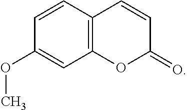

Herniarin is often found in Herniaria glabra, Ayanpana triplinervis, and in species of the genus Prunus (P. mahaleb, P. pensylvanica, and P. maximowiczil).

As used herein, the term “hexanaldehyde” refers to a compound with the following structural formula:

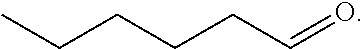

Hexanaldehyde is often characterized as having a grassy odor. Hexanaldehyde is also called hexanal.

As used herein, the term “hexanoic acid” refers to a compound with the

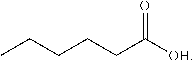

Hexanoic acid is often characterized as having a pungent, acrid, sour, fatty, sweaty, rancid cheese odor.

As used herein, the term “alpha-humulene” refers to a compound with the following structural formula:

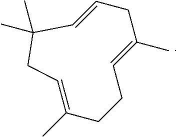

Alpha-humulene is often characterized as contributing to the flavor profile of beer. Alpha-humulene is sometimes often referred to as simply “Humulene” or “Caryophyllene” and in the context of this disclosure both may be used interchangeably.

As used herein, the term “beta-humulene” refers to a compound with the following structural formula:

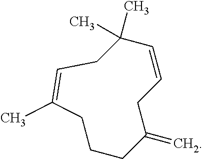

Beta-humulene is often characterized as having a green aroma.

As used herein, the term “ionol” refers to a compound with the following structural formula:

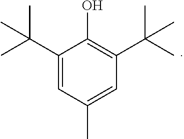 [SMILES: Cc1cc(C(C)(C)C)c(O)c(C(C)(C)C)c1]

Ionol is also known as Butylated Hydroxy Toluene (BHT). For the purpose of this disclosure “ionol” also refers to 3-oxo-alpha-ionol and/or beta-ionol in a pure and/or mixture of any ratio. Ionol is often characterized as having a sweet, woody, herbal, fruity, floral, tropical, and berry aroma.

As used herein, the term “ionone” refers to either or both alpha-ionone and/or beta-ionone as pure forms or mixtures in any ratio.

Alpha-ionone refers to the following structural formula:

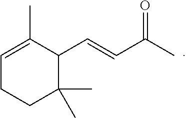

Beta-ionone refers to the following structural formula:

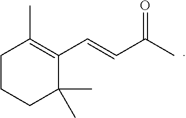

Alpha-ionone and beta-ionone as a mixture have an aroma similar to the violet flower.

As used herein, the term “ipsdienol” refers to a compound with the

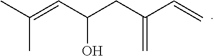

Ipsdienol is often characterized as having a balsamic, piney aroma.

As used herein, the term “isoamyl acetate” refers to a compound with the following structural formula:

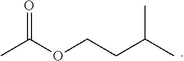

Isoamyl acetate is often characterized as having an odor similar to banana and pears.

As used herein, the term “isoamyl alcohol” refers to a compound with the following structural formula:

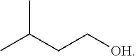

Isoamyl alcohol is often characterized as having a pungent, fermented, fusel, alcohol-like, aroma with fruity, banana and molasses notes. Isoamyl alcohol is used as a flavor and fragrance agent.

As used herein, the term “isoamyl formate” refers to a compound with the following structural formula:

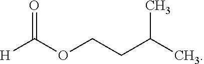

Isoamyl formate is often characterized as having a vinous, dry, earthy, fruity, green, plum, and blackcurrant aroma.

As used herein, the term “isoborneol” refers to a compound with the following structural formula:

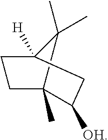

Isoborneol is often characterized as having a camphoraceous, sweet & musty aroma.

As used herein, the term “isomyrcenol” refers to a compound with the following structural formula:

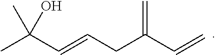

Isomyrcenol is often characterized as having a fresh, floral, lime-like odor. Isomyrcenol is used in the chemical communication systems of beetles.

As used herein, the term “isopulegol” refers to a compound with the

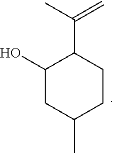

Isopulegol is often characterized as having a medium strength odor that is minty, cooling and bittersweet. Within the context of this disclosure, “isopulegol” may also refer to any number of isomeric forms.

As used herein, the term “isovaleric acid” refers to a compound with the following structural formula:

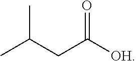

Isovaleric acid is often characterized as having a strong, pungent, sour, and sweaty cheese smell. Isovaleric acid is also known as 3-methylbutanoic acid.

As used herein, the term “isoprene” refers to a compound with the following structural formula:

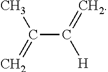

Isoprene is often considered as a building block for many other terpenes.

As used herein, the term “kahweol” refers to a compound with the

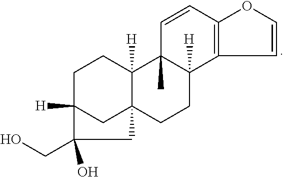

As used herein, the term “lavandulol” refers to either the R and S enantiomers as pure forms or mixture in any ratio with the following structural formula:

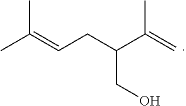

Lavandulol is often found in the essential oil of lavender.

As used herein, the term “limonene” refers to a terpene with the following structural formula:

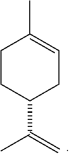

Limonene is often characterized as having a smell similar to oranges and other citrus fruits. Within the context of this disclosure, the term “Limonene” encompasses all possible enantiomers and isomers of the compound in as either individual compounds or in a racemic mixture.

As used herein, the term “gamma-linolenic acid” refers to a compound with the following structural formula:

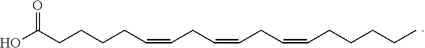 [SMILES: CCCCC/C=C\C/C=C\C/C=C\CCCCC(=O)O]

Gamma-linolenic acid is often characterized as an unsaturated fatty acid found primarily in vegetable oils.

As used herein, the term “linalool” refers to a terpene with the following structural formula:

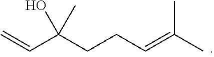

Linalool has two known enantiomeric forms. (S)-(+)-Linalool is often characterized as sweet and floral and the (R)-form is more woody and lavender-like. Within the context of this disclosure, the “Linalool” refers to either of the enantiomers or a racemic mixture of the two.

As used herein, the term “longifolene” refers to a compound having both (+) and (−) enantiomers. The (+) enantiomer refers to the following structural formula:

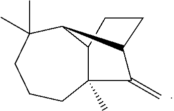

Within the context of this disclosure, the term “longifolene” refers to either of its (+) and/or (−) enantiomers in a pure form or mixture in any ratio. Longifolene is often characterized as having a sweet, woody, rosy, medical, fir needle odor.

As used herein, the term “alpha-longipinene” refers to a compound with the following structural formula:

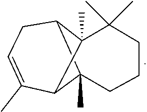 [SMILES: CC1=CCC2[C@]3(C)CCCC(C)(C)[C@@]2(C)C13]

Alpha-longipinene is often characterized as having a pine aroma.

As used herein, the term “lycopene” refers to a compound with the following structural formula:

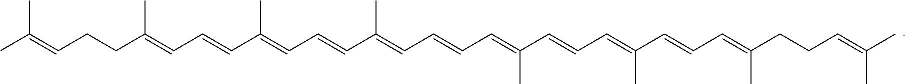 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

Lycopene is often characterized for its red color.

As used herein, the term “luteolin” refers to a compound with the structural formula:

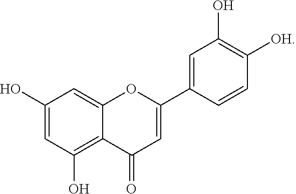 [SMILES: O=c1cc(-c2ccc(O)c(O)c2)oc2cc(O)cc(O)c12]

Luteolin is often found in leaves, rinds, barks, clover blossoms, and ragweed pollen.

As used herein, the term “menthol” refers to a compound with the following structural formula:

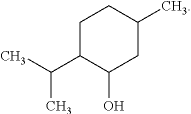

Menthol is often characterized as having a cooling, minty, peppermint aroma and flavor. Menthol is obtained from cornmint, peppermint or other mint oils.

As used herein, the term “methyl butyrate” refers to a compound with the following structural formula:

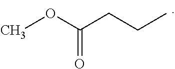

Methyl butyrate is often characterized as having a sulfurous odor.

As used herein, the term “3-Mercapto-2-Methylpentanal” refers to a compound with the following structural formula:

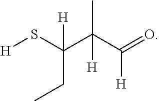

3-Mercapto-2-Methylpentanal is often characterized as having a sulfurous, alliaceous, garlic odor.

As used herein, the term “3-Mercapto-2-Methylpentanol” refers to a compound with the following structural formula:

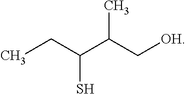

3-Mercapto-2-Methylpentanol is often characterized as having a very strong, sulfurous, onion type aroma.

As used herein, the term “mercaptan” refers to an organosulfur compound containing a carbon-bonded sulfhydryl. Mercaptan is often characterized as the main odor constituent added to assist in the detection of natural gas (which in pure form is odorless), and the “smell of natural gas” is due to the smell of the mercaptan thiol used as the odorant. Mercaptan is also referred to as thiol.

As used herein, the term “beta-mercaptoethanol” refers to a compound with the following structural formula:

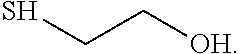

Beta-mercaptoethanol is often characterized as having an extremely strong and persistent aroma of sulfur.

As used herein, the term “mercaptoacetic acid” refers to a compound with the following structural formula:

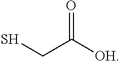

Mercaptoacetic acid is often characterized as having an ugly, sharp, acrid, skunk smell with plenty of endurance. Mercaptoacetic acid is also referred to as thioglycolic acid.

As used herein, the term “allyl mercaptan” refers to a compound with the following structural formula:

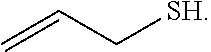

Allyl mercaptan is often characterized as having a strong sulfurous and alliaceous aroma.

As used herein, the term “benzyl mercaptan” refers to a compound with the following structural formula:

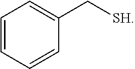

Benzyl mercaptan is often characterized as having a strong, sharp, alliaceous, sulfurous, onion, garlic, and horseradish aroma.

As used herein, the term “butyl mercaptan” refers to a compound with the following chemical formula C₄H₁₀S. In one embodiment, the butyl mercaptan has the following structural formula:

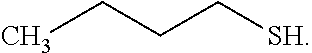

In one embodiment, the butyl mercaptan has the following structural formula:

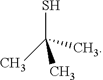

Butyl mercaptan is often characterized as having an extremely strong foul-smelling odor, commonly described as “skunk” odor.

As used herein, the term “methyl mercaptan” refers to a compound with the following structural formula:

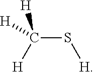

Methyl mercaptan is often characterized as having an extremely strong, sulfurous, decomposing cabbage, skunky aroma. Methyl mercaptan is also called methanethiol.

As used herein, the term “furfuryl mercaptan” refers to a compound with the following structural formula:

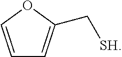

Furfuryl mercaptan is often characterized as having a very strong, oily, fatty, sulfurous, skunky odor.

As used herein, the term “ethylene mercaptan” refers to a compound with the following structural formula:

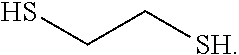

Ethylene mercaptan is often characterized as having a very high strength, sulfurous, skunky odor.

As used herein, the term “propyl mercaptan” refers to a compound with the following structural formula:

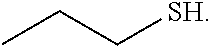

Propyl mercaptan is often characterized as having a high strength, alliaceous, natural gas, sweet onion, cabbage odor. Propyl mercaptan is also referred to as propanethiol.

As used herein, the term “thenyl mercaptan” refers to a compound with the following structural formula:

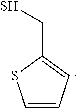

Thenyl mercaptan is often characterized as having a strong roast coffee, fishy aroma. Thenyl mercaptan is used as a flavoring agent mimicking the flavor of coffee.

As used herein, the term “methyl salicylate” refers to a compound with the

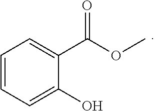

Methyl salicylate is often characterized as having a strong, distinct wintergreen aroma. Methyl salicylate is used as a fragrance agent, a food and beverage agent.

As used herein, the term “methylbutenol” refers to a compound with the following structural formula:

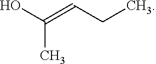

Methylbutenol is often characterized as having a very strong, malty herb aroma. Methylbutenol is released in high concentrations from lodgepole pine trees.

As used herein, the term “methyl-2-methylvalerate” refers to a carboxylic acid with the following structural formula:

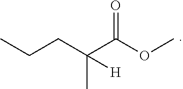

Methyl-2-methylvalerate is often characterized as having a fruity, sweet, berry, pineapple, apple, banana, green melon, and tropical aromas.

As used herein, the term “methyl thiobutyrate” refers to a compound with the following structural formula:

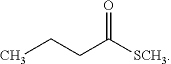

Methyl thiobutyrate is often characterized as a flavoring and food agent.

As used herein, the term “beta-myrcene” refers to a terpene with the following structural formula:

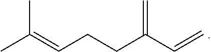

Beta-Myrcene is often characterized as having an earthy, fruity clove-like odor. Beta-myrcene is also referred to as “myrcene”.

As used herein, the term “gamma-muurolene” refers to a compound with the following structural formula:

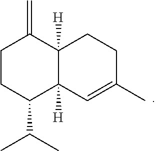

Gamma-muurolene is often characterized as having an herbal, woody, and spicy aroma.

As used herein, the term “nepetalactone” refers to a compound with the

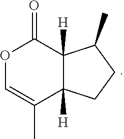

Nepetalactone is often characterized as having a valerian odor. Nepetalactone is also one of the main attractants and stimulants in catnip, which, when inhaled, insights wild play behavior in many cats.

As used herein, the term “nerol” refers to a compound with the following structural formula:

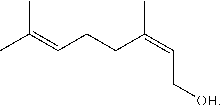

Nerol is often characterized a constituent of fragrances in cosmetics and as a flavor agent.

As used herein, the term “nerolidol” refers to a compound with either of the following structural formulas or any mixture thereby:

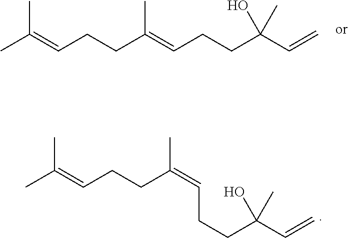

Nerolidol is often characterized as having a woody aroma, similar to fresh bark. Within the context of this disclosure, the term “Nerolidol” refers to either or both of the cis and trans isomers.

As used herein, the term “neryl acetate” refers to a compound with the following structural formula:

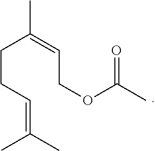

Neryl acetate is often characterized as having a floral and fruity aroma.

As used herein, the term “nonanaldehyde” refers to a compound with the following structural formula:

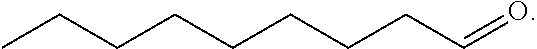

Nonanaldehyde is often characterized as having a soapy, citrus odor. Nonanaldehyde is a component of perfumes, although it also occurs in several natural oils. Nonanaldehyde is also referred to as nonanal.

As used herein, the term “nonanoic acid” refers to a carboxylic acid with the following structural formula:

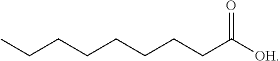

Nonanoic acid is often characterized as having an unpleasant, rancid, waxy, dirty, and cheesy aroma.

As used herein, the term “ocimene” refers to a compound with one of the isomeric forms:

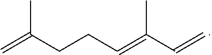

Within the context of this disclosure, ocimene refers to any of the isomers in a single pure form or a mixture in any ratio.

As used herein, the term “octanal” refers to a compound with the following structural formula:

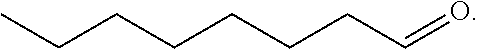

Octanal is often characterized as having a very strong waxy, citrus, orange peel aroma.

As used herein, the term “octanoic acid” refers to a compound with the following structural formula:

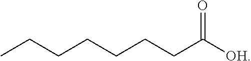

Octanoic acid is often characterized as having a musty, rancid, cheesy, sweat-like odor Octanoic acid is used as a cosmetic surfactant and as a flavoring agent for processed cheeses. Octanoic acid is also referred to as caprylic acid.

As used herein, the term “p-cymene” refers to a compound with the

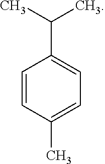

Within context of this disclosure, Cymene may refer to p-Cymene or any other isomeric form, e.g., m-Cymene or o-Cymene.

As used herein, the term “pentyl butyrate” refers to a compound with the following structural formula:

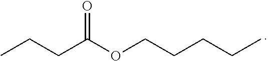

Pentyl butyrate is often characterized as having a smell reminiscent of pear or apricot.

As used herein, the term “phellandrene” refers to either of the two structural formulas:

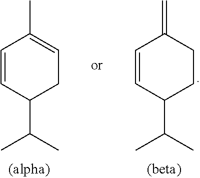

Alpha-phellandrene is often characterized as having a citrus, herbal, black pepper-like odor. Beta-phellandrene is often characterized as having a peppery-minty and slightly citrusy odor with a slight minty-turpentine note.

As used herein, the term “phenylacetaldehyde” refers to a compound with the following structural formula:

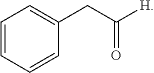

Phenylacetaldehyde is often characterized as having a strong pungent honey-like sweet and green floral odor. Phenylacetaldehyde is found in buckwheat, chocolate, and many other foods and flowers.

As used herein, the term “phenylethanethiol” refers to a compound with the following chemical formula: C₈H₁₀S comprising of multiple isomers.

Within the context of this disclosure, phenylethanethiol refers to any of the isomers as a pure compound and/or in any mixture. Phenylethanethiol is often characterized as having the high strength odor of rubber.

As used herein, the term “phenylacetic acid” refers to a compound with the following structural formula:

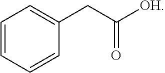

Phenylacetic acid is often characterized as having a sour yet sweet, waxy, civet, honey, rosey, floral odor similar to honeysuckle with notes of chocolate and tobacco. Phenylacetic acid is also used in cosmetics for perfuming.

As used herein, the term “phytol” refers to a compound with the following structural formula:

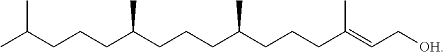 [SMILES: C/C(=C\CO)CCC[C@H](C)CCC[C@H](C)CCCC(C)C]

Phytol is often characterized having a mild floral, balsamic, and green tea type of aroma.

As used herein, the term “alpha-pinene” refers to a compound with either of the following structural formulas:

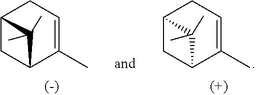

Alpha-Pinene is often characterized as having a pine tree like aroma.

As used herein, the term “beta-pinene” refers to a terpene with the following structural formula:

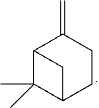

Beta-Pinene is often characterized as having a woody-green pine-like smell. Beta-Pinene is one of the most abundant compounds released by forest trees. Beta-Pinene is an isomer of pinene.

As used herein the term “pristimerin” refers to a compound with the

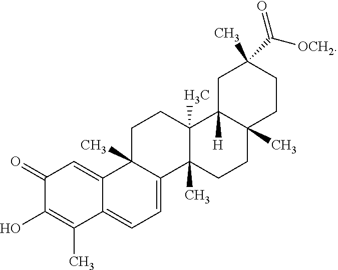

Pristimerin is often characterized as having antiviral and antitumor properties. Pristimerin is also called celastrol methyl ester celastrol.

As used herein, the term “pulegone” refers to a compound with the following structural formula:

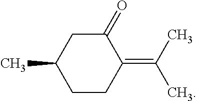

Pulegone is often characterized as having a smell similar to peppermint.

As used herein, the term “quercetin” refers to a compound with the following structural formula:

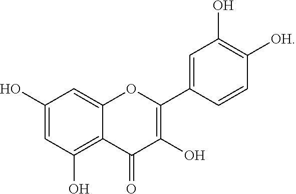 [SMILES: O=c1c(O)c(-c2ccc(O)c(O)c2)oc2cc(O)cc(O)c12]

As used herein, the term “retinol” refers to a compound with the following structural formula:

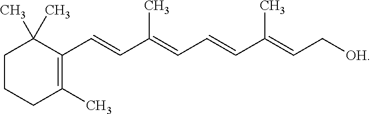 [SMILES: CC1=C(/C=C/C(C)=C/C=C/C(C)=C/CO)C(C)(C)CCC1]

Retinol is often characterized as a type of vitamin A that can be converted to other types of vitamin A.

As used herein, the term “rutin” refers to a compound with the following structural formula:

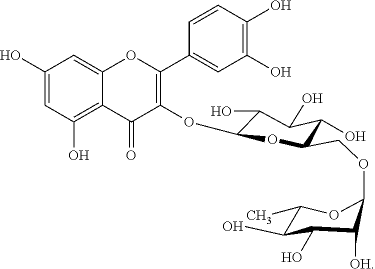

Rutin is often characterized as citrus flavonoid and found in many plants.

As used herein, the term “sabinene” refers to a compound with the following structural formula:

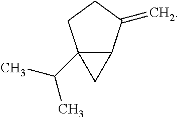

Sabinene is often characterized as having a peppery, woody, herbaceous, and spicy pine odor with citrus notes. Sabinene is found in oak trees, tea tree oil, black pepper and is a major constituent of carrot seed oil. Within the context of this disclosure, the term sabinene refers to either a enantiomeric form or a mixture in any ratio.

As used herein, the term “sabinene hydrate” refers to a compound with the following structural formula:

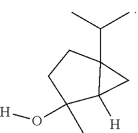

Sabinene hydrate is often characterized as having an herbal, minty, eucalyptol, and terpy odor with a spicy nuance.

As used herein, the term “cis-sabinene hydrate” refers to a compound with the following structural formula.

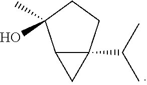

Cis-sabinene hydrate is often characterized as having a balsamic odor.

As used herein, the term “trans-sabinene hydrate” refers to the following structural formula.

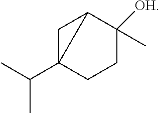

Trans-sabinene hydrate is often characterized as having a woody, balsamic odor.

As used herein, the term “safranal” refers to a compound the following structural formula:

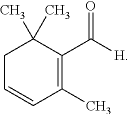

Safranal is often characterized as the component primarily responsible for the aroma of saffron.

As used herein, the term “alpha-selinene” refers to a compound with the following structural formula:

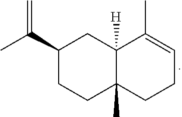

Alpha-selinene is often characterized as having a distinct odor of amber. Alpha-selinene is one of the principal components of the oil from celery seeds.

As used herein, the term “alpha-sinensal” refers to a compound with the following structural formula:

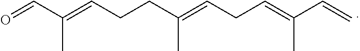 [SMILES: C=C/C(C)=C/C/C=C(\C)CC/C=C(\C)C=O]

Alpha-sinensal is often characterized as having a citrus orange, mandarin aroma.

As used herein, the term “beta-sinensal” refers to a compound with the following structural formula:

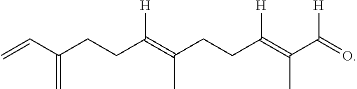 [SMILES: C=CC(=C)CC/C=C(\C)CC/C=C(\C)C=O]

Beta-sinensal is often characterized as having a sweet, fresh, waxy, and citrus odor. Beta-sinensal is used in fragrances.

As used herein, the term “beta-sitosterol” refers to a compound with the following structural formula:

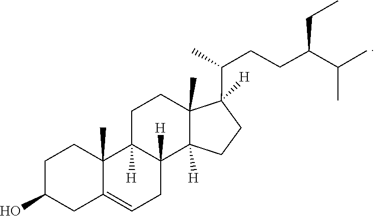

Beta-sitosterol is found in pecans, avocados, pumpkin seeds, cashews, corn oils, and soybeans.

As used herein, the term “squalene” refers to a compound with the following structural formula:

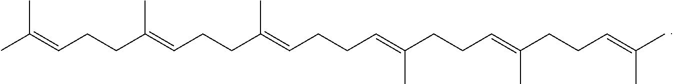 [SMILES: CC(C)=CCC/C(C)=C/CC/C(C)=C/CC/C=C(\C)CC/C=C(\C)CCC=C(C)C]

Squalene is often characterized as playing an important part in the synthesis of plant and animal sterols.

As used herein, the term “taxadiene” refers to a compound with the following structural formula:

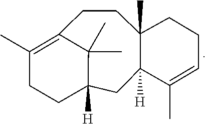 [SMILES: CC1=CCC[C@@]2(C)CCC3=C(C)CC[C@@H](C[C@H]12)C3(C)C]

Taxadiene is an intermediate in the synthesis of taxol.

As used herein, the term “terpin” refers to a compound of the following structural formula:

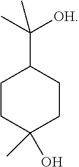

As used herein the term “terpineol” refers to a compound with the following structural formula:

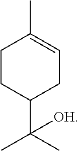

Within the context of this disclosure, terpineol includes any of the isomeric forms either as a single form or a mixture in any ratio. Terpineol is used extensively in the perfume industry. Within the context of this disclosure, terpineol refers to any and all isomeric forms, alpha, beta, gamma, etc, in any ratio or combination,

As used herein, the term “terpinen-4-ol” refers to the isomer of terpineol that has the following structural formula:

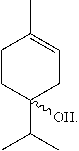

Terpinen-4-ol is often characterized as having an herbaceous, peppery, woody odor and is used in commercial fragrances. Terpinen-4-ol is considered the primary active ingredient of tea tree oil.

As used herein, the term “alpha-terpinene” refers a compound the following structural formula:

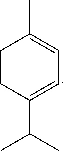

Alpha-terpinene is often characterized as having a lemony-citrus aroma. Alpha-terpinene has been isolated from cardamom and marjoram oils.

As used herein, the term “gamma-terpinene” refers to a compound with the

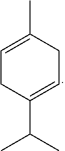

Gamma-terpinene is often characterized as having an herbaceous, citrusy sweet aroma.

As used herein, the term “terpinolene” or “delta-terpinene” refers to a compound with the following structural formula:

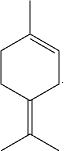

Terpinolene is often characterized as having an herbal aroma.

Terpinolene's flavor has been described as sweet, woody, lemon, and lime-like.

As used herein, the term “thiophenol” refers to a compound with the following structural formula:

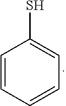

Thiophenol is often characterized as having a sulfurous odor.

As used herein, the term “thujone” refers to a compound with either of the following structural formulas:

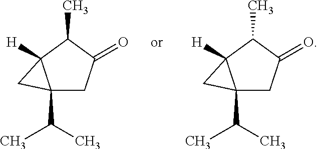

Within the context of this closure, the term “thujone” refers to any of the isomeric forms in a pure form or mixture of any ratio. Thuj one is often characterized as having a menthol like odor.

As used herein, the term “thymol” refers to a compound with the following structural formula:

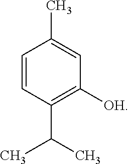

Thymol is often found in oil of thyme.

As used herein, the term “alpha-tocopherol” refers to a compound with the

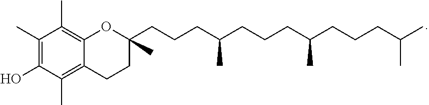 [SMILES: Cc1c(C)c2c(c(C)c1O)CC[C@@](C)(CCC[C@H](C)CCC[C@H](C)CCCC(C)C)O2]

Alpha-tocopherol is often characterized as having a very bland, slight, vegetable oil-like odor.

As used herein, the term “tonka undecanone” refers to a compound with the following structural formula:

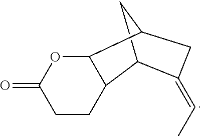

Tonka undecanone is often characterized as having a sweet, spicy, balsamic, and Tonka woody tobacco aroma.

As used herein, the term “undecanal” refers to an aldehyde that has the following structural formula:

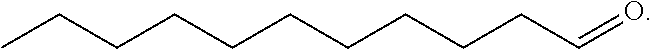

Undecanal is often characterized as having a soapy, aldehydic, waxy, and slightly effervescent orangey citrus-like odor with a watermelon, pineapple and cilantro background. Undecanal is a common component of perfumes.

As used herein, the term “valeraldehyde” or “pentanal” refers to a compound with the following structural formula:

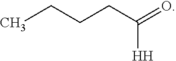

Valeraldehyde is often characterized as having a fermented type, diffusive, bready, fruity odor with berry nuances. Used as a flavoring agent, valeraldehyde has a wine-like, fermented, bready, cocoa flavor with chocolate notes.

As used herein, the term “verdoxan” refers to a cyclic ether with the following structural formula:

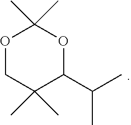

Verdoxan is often characterized as having a medium strength odor of earthy, fruity, green, herbal, sawn old wood. Verdoxan is used in fragrances and cosmetics.

As used herein, the term “alpha-ylangene” refers to a compound with the following structural formula:

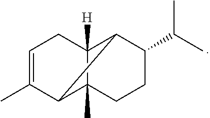

Alpha-ylangene is often characterized as having a spicy, fruity aroma. Alpha-ylangene is a natural substance often found in wine and is emitted by some plants.

As used herein, the term “umbelliferone” refers to a compound with the following structural formula:

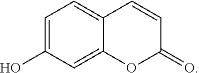 [SMILES: O=c1ccc2ccc(O)cc2o1]

As used herein, the term “vanillin” refers to a compound with the following structural formula:

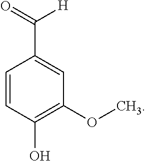 [SMILES: COc1cc(C=O)ccc1O]

Vanillin is often characterized as having a pleasant, sweet aroma, and the characteristic vanilla-like flavor.

As used herein, the term “flavonoid” means a class of secondary plant metabolites that have a general 15-carbon skeleton structure which consists of two phenyl rings and a heterocyclic ring. Examples of flavonoids within the context of this disclosure include broadly phenolic acids, stilbenoids, dihydroflavonols, anthocyanins, anthocyanidins, polyphenols, tannins, flavones, flavan-3-ols, Flavan-4-ol, Flavan-3,4-diol flavonols, phytochemicals, antioxidants, homoisoflavonoids, and any keytone containing compound including: flavonoids or bioflavonoids, isoflavonoids, neoflavonoids or their derivatives.

Disclosed herein, is a composition comprising a purified cannabinoid and a purified flavonoid.

In one embodiment, the composition herein disclosed comprises a flavonoid chosen from phenolic acids, stilbenoids, dihydroflavonols, anthocyanins, anthocyanidins, polyphenols, tannins, flavones, flavan-3-ols, Flavan-4-ol, Flavan-3,4-diol flavonols, phytochemicals, antioxidants, homoisoflavonoids, phenylpropanoids, Phloroglucinols coumarins, Naphthodianthrones, Steroid glycosides, bioflavonoids, isoflavonoids, and neoflavonoids. In one embodiment, the composition herein disclosed comprises a flavonoid chosen from Adenosine, Adhyperforin, amentoflavone, Anandamide, Apigenin, Cannaflavin B, Catechin (C), Catechin 3-gallate (Cg), Chlorogenic acid, cichoric acid, caftaric acid, Daidzein, Delphinidin, Eleutherosides, Epicatechin 3-gallate (ECg), Epicatechins, Epicatechin, epigallocatechin, myricetin, Oxalic acid, Pelargonidin, Tannin, Theaflavin-3-gallate, Theanine, Theobromine, Theophylline, Tryptophan, Tyramine, Xanthine, Caffeine, Cannaflavin A, Cannaflavin B, Catechin (C), Catechin 3-gallate (Cg), Epicatechin 3-gallate (ECg), Epicatechins (Epicatechin (EC)), epigallocatechin, Epigallocatechin (EGC), Epigallocatechin 3-gallate (EGCg), Gallocatechin (GC), Gallocatechin 3-gallate (GCg)), Gamma amino butyric acid, Genistein, Ginkgo biloba, Ginsenosides, Quercetin, Quercitrin, or Rutin. In one embodiment, the composition herein disclosed comprises a flavonoid chosen from Caffeine, Cannaflavin A, Cannaflavin B, Catechin (C), Catechin 3-gallate (Cg), Epicatechin 3-gallate (ECg), Epicatechins (Epicatechin (EC)), epigallocatechin, Epigallocatechin (EGC), Epigallocatechin 3-gallate (EGCg), Gallocatechin (GC), Gallocatechin 3-gallate (GCg)), Gamma amino butyric acid, Genistein, Ginkgo biloba, Ginsenosides, Quercetin, Quercitrin, or Rutin.

As used herein, the term “mineral” means a naturally occurring substance that is solid, inorganic, and has an ordered atomic structure. Examples of minerals within the context of this disclosure include lithium.

Disclosed herein are compositions comprising a purified cannabinoid and a purified mineral. In one embodiment, the purified mineral is lithium. In one embodiment, the composition consists essentially of a first purified cannabinoid and a purified mineral.

Disclosed herein are new compositions comprising unique combinations of a first purified cannabinoid and a first purified terpene. In one embodiment, the first purified cannabinoid and first purified terpene are present in non-naturally occurring concentrations. In one embodiment, the first purified cannabinoid and first purified terpene are present in non-naturally occurring ratios. In one embodiment, the compositions disclosed herein provide particular benefits previously unavailable with naturally occurring cannabinoid and/or terpene profiles, such as those found in plants, harvested flowers, extracts, or conventional products derived from the same.

In one embodiment, the said combination of a first purified cannabinoid and a first purified terpene provide a synergistic effect at a mammalian cellular receptor, such as a G protein coupled receptor. In one embodiment, the receptor is CB1. In one embodiment, the receptor is CB2. In one embodiment, the receptor is GPR55. In one embodiment, the receptor is 5HT-1A. In one embodiment, the receptor is 5HT-2A. In one embodiment, the receptor is TRPV1. In one embodiment, the receptor is an opioid receptor, e.g., μ-opioid receptor.

Disclosed herein is a composition comprising:
a first purified cannabinoid, chosen from THC, D9-THC, D8-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA; and
a first purified terpene chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol.

Within the context of this disclosure, using “combination of a first purified cannabinoid and a first purified terpene” may be accomplished by a formulating a homogeneous mixture prior to consuming (e.g., combusting, vaporizing, inhaling, or ingesting) the mixture.

In one embodiment, the compositions disclosed herein are combined with a carrier oil.

As used herein, the term “carrier oil” refers to a substance facilitating the administration of the compositions disclosed herein. In one embodiment, the carrier oil is grapeseed oil. In one embodiment, the carrier oil is coconut oil. In one embodiment, the carrier oil is polyethylene glycol.

As used herein, the term “polyethylene glycol” refers to a compound with the following structural formula:

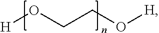

wherein n is an integer.

Polyethylene glycol is also known as PEG, polyethylene oxide (PEO), and polyoxyethylene (POE).

In one embodiment, the purified components disclosed herein are purposefully chosen for a desired effect. In one embodiment, the purposefully chosen purified components are then combined with a carrier oil. In a further embodiment, the purposefully purified components in the carrier oil is combined with a food product. In a further embodiment, the purposefully purified components in the carrier oil is combined with a beverage product. In a further embodiment, the purposefully purified components in the carrier oil is combined with a personal care product, e.g., topical cream, soap, shampoo, etc. In a further embodiment, the purposefully purified components in the carrier oil is combined with a drug. In a further embodiment, the purposefully purified components in the carrier oil is combined with a plant extract. In a further embodiment, the purposefully purified components in the carrier oil is used with a device, e.g., vaporizer, intravenous drug, etc. In a further embodiment, the purposefully purified components in the carrier oil is used in combination with any of the above-mentioned embodiments.

In one example, particular purified components, such as purified terpenes, may be added to purified or unpurified cannabinoids to create the compositions disclosed herein at the time of use.

Within the context of this disclosure the term “use” includes consuming via ingestion or heating and inhaling by, for example, combusting or vaporizing.

In one example, particular purified components, such as purified terpenes, may be added to purified or unpurified cannabinoids to create the compositions disclosed herein at the time of use. Within the context of this disclosure the term “use” includes consuming via ingestion or heating and inhaling by, for example, combusting or vaporizing.

In one embodiment, the first purified cannabinoid is THC and the first purified terpene is Limonene.

In one embodiment, the first purified cannabinoid is THC and the first purified terpene is Nerolidol.

In one embodiment, the first purified cannabinoid is THC and the first purified terpene is Beta-Myrcene.

In one embodiment, the first purified cannabinoid is THC and the first purified terpene is Linalool.

In one embodiment, the first purified cannabinoid is THC and the first purified terpene is Alpha-Caryophyllene.

In one embodiment, the first purified cannabinoid is THC and the first purified terpene is Beta-Caryophyllene.

In one embodiment, the first purified cannabinoid is THC and the first purified terpene is Alpha-Pinene.

In one embodiment, the first purified cannabinoid is THC and the first purified terpene is Beta-Pinene.

In one embodiment, the first purified cannabinoid is THC and the first purified terpene is Alpha-Bisabolol.

In one embodiment, the first purified cannabinoid is THC and the first purified terpene is Delta-3-Carene.

In one embodiment, the first purified cannabinoid is THC and the first purified terpene is Borneol.

In one embodiment, the first purified cannabinoid is THC and the first purified terpene is Eucalyptol.

In one embodiment, the first purified cannabinoid is THC and the first purified terpene is p-Cymene.

In one embodiment, the first purified cannabinoid is THC and the first purified terpene is Alpha-Terpineol.

In one embodiment, the first purified cannabinoid is THC and the first purified terpene is Alpha-Humulene.

In one embodiment, the first purified cannabinoid is THC and the first purified terpene is Terpinolene.

In one embodiment, the first purified cannabinoid is THC and the first purified terpene is Pulegone.

In one embodiment, the first purified cannabinoid is THC and the first purified terpene is Camphene.

In one embodiment, the first purified cannabinoid is THC and the first purified terpene is Geraniol.

In one embodiment, the first purified cannabinoid is D9-THC and the first purified terpene is Limonene.

In one embodiment, the first purified cannabinoid is D9-THC and the first purified terpene is Nerolidol.

In one embodiment, the first purified cannabinoid is D9-THC and the first purified terpene is Beta-Myrcene.

In one embodiment, the first purified cannabinoid is D9-THC and the first purified terpene is Linalool.

In one embodiment, the first purified cannabinoid is D9-THC and the first purified terpene is Alpha-Caryophyllene.

In one embodiment, the first purified cannabinoid is D9-THC and the first purified terpene is Beta-Caryophyllene.

In one embodiment, the first purified cannabinoid is D9-THC and the first purified terpene is Alpha-Pinene.

In one embodiment, the first purified cannabinoid is D9-THC and the first purified terpene is Beta-Pinene.

In one embodiment, the first purified cannabinoid is D9-THC and the first purified terpene is Alpha-Bisabolol.

In one embodiment, the first purified cannabinoid is D9-THC and the first purified terpene is Delta-3-Carene.

In one embodiment, the first purified cannabinoid is D9-THC and the first purified terpene is Borneol.

In one embodiment, the first purified cannabinoid is D9-THC and the first purified terpene is Eucalyptol.

In one embodiment, the first purified cannabinoid is D9-THC and the first purified terpene is p-Cymene.

In one embodiment, the first purified cannabinoid is D9-THC and the first purified terpene is Alpha-Terpineol.

In one embodiment, the first purified cannabinoid is D9-THC and the first purified terpene is Alpha-Humulene.

In one embodiment, the first purified cannabinoid is D9-THC and the first purified terpene is Terpinolene.

In one embodiment, the first purified cannabinoid is D9-THC and the first purified terpene is Pulegone.

In one embodiment, the first purified cannabinoid is D9-THC and the first purified terpene is Camphene.

In one embodiment, the first purified cannabinoid is D9-THC and the first purified terpene is Geraniol.

In one embodiment, the first purified cannabinoid is D8-THC and the first purified terpene is Limonene.

In one embodiment, the first purified cannabinoid is D8-THC and the first purified terpene is Nerolidol.

In one embodiment, the first purified cannabinoid is D8-THC and the first purified terpene is Beta-Myrcene.

In one embodiment, the first purified cannabinoid is D8-THC and the first purified terpene is Linalool.

In one embodiment, the first purified cannabinoid is D8-THC and the first purified terpene is Alpha-Caryophyllene.

In one embodiment, the first purified cannabinoid is D8-THC and the first purified terpene is Beta-Caryophyllene.

In one embodiment, the first purified cannabinoid is D8-THC and the first purified terpene is Alpha-Pinene.

In one embodiment, the first purified cannabinoid is D8-THC and the first purified terpene is Beta-Pinene.

In one embodiment, the first purified cannabinoid is D8-THC and the first purified terpene is Alpha-Bisabolol.

In one embodiment, the first purified cannabinoid is D8-THC and the first purified terpene is Delta-3-Carene.

In one embodiment, the first purified cannabinoid is D8-THC and the first purified terpene is Borneol.

In one embodiment, the first purified cannabinoid is D8-THC and the first purified terpene is Eucalyptol.

In one embodiment, the first purified cannabinoid is D8-THC and the first purified terpene is p-Cymene.

In one embodiment, the first purified cannabinoid is D8-THC and the first purified terpene is Alpha-Terpineol.

In one embodiment, the first purified cannabinoid is D8-THC and the first purified terpene is Alpha-Humulene.

In one embodiment, the first purified cannabinoid is D8-THC and the first purified terpene is Terpinolene.

In one embodiment, the first purified cannabinoid is D8-THC and the first purified terpene is Pulegone.

In one embodiment, the first purified cannabinoid is D8-THC and the first purified terpene is Camphene.

In one embodiment, the first purified cannabinoid is D8-THC and the first purified terpene is Geraniol.

In one embodiment, the first purified cannabinoid is THCA and the first purified terpene is Limonene.

In one embodiment, the first purified cannabinoid is THCA and the first purified terpene is Nerolidol.

In one embodiment, the first purified cannabinoid is THCA and the first purified terpene is Beta-Myrcene.

In one embodiment, the first purified cannabinoid is THCA and the first purified terpene is Linalool.

In one embodiment, the first purified cannabinoid is THCA and the first purified terpene is Alpha-Caryophyllene.

In one embodiment, the first purified cannabinoid is THCA and the first purified terpene is Beta-Caryophyllene.

In one embodiment, the first purified cannabinoid is THCA and the first purified terpene is Alpha-Pinene.

In one embodiment, the first purified cannabinoid is THCA and the first purified terpene is Beta-Pinene.

In one embodiment, the first purified cannabinoid is THCA and the first purified terpene is Alpha-Bisabolol.

In one embodiment, the first purified cannabinoid is THCA and the first purified terpene is Delta-3-Carene.

In one embodiment, the first purified cannabinoid is THCA and the first purified terpene is Borneol.

In one embodiment, the first purified cannabinoid is THCA and the first purified terpene is Eucalyptol.

In one embodiment, the first purified cannabinoid is THCA and the first purified terpene is p-Cymene.

In one embodiment, the first purified cannabinoid is THCA and the first purified terpene is Alpha-Terpineol.

In one embodiment, the first purified cannabinoid is THCA and the first purified terpene is Alpha-Humulene.

In one embodiment, the first purified cannabinoid is THCA and the first purified terpene is Terpinolene.

In one embodiment, the first purified cannabinoid is THCA and the first purified terpene is Pulegone.

In one embodiment, the first purified cannabinoid is THCA and the first purified terpene is Camphene.

In one embodiment, the first purified cannabinoid is THCA and the first purified terpene is Geraniol.

In one embodiment, the first purified cannabinoid is THCV and the first purified terpene is Limonene.

In one embodiment, the first purified cannabinoid is THCV and the first purified terpene is Nerolidol.

In one embodiment, the first purified cannabinoid is THCV and the first purified terpene is Beta-Myrcene.

In one embodiment, the first purified cannabinoid is THCV and the first purified terpene is Linalool.

In one embodiment, the first purified cannabinoid is THCV and the first purified terpene is Alpha-Caryophyllene.

In one embodiment, the first purified cannabinoid is THCV and the first purified terpene is Beta-Caryophyllene.

In one embodiment, the first purified cannabinoid is THCV and the first purified terpene is Alpha-Pinene.

In one embodiment, the first purified cannabinoid is THCV and the first purified terpene is Beta-Pinene.

In one embodiment, the first purified cannabinoid is THCV and the first purified terpene is Alpha-Bisabolol.

In one embodiment, the first purified cannabinoid is THCV and the first purified terpene is Delta-3-Carene.

In one embodiment, the first purified cannabinoid is THCV and the first purified terpene is Borneol.

In one embodiment, the first purified cannabinoid is THCV and the first purified terpene is Eucalyptol.

In one embodiment, the first purified cannabinoid is THCV and the first purified terpene is p-Cymene.

In one embodiment, the first purified cannabinoid is THCV and the first purified terpene is Alpha-Terpineol.

In one embodiment, the first purified cannabinoid is THCV and the first purified terpene is Alpha-Humulene.

In one embodiment, the first purified cannabinoid is THCV and the first purified terpene is Terpinolene.

In one embodiment, the first purified cannabinoid is THCV and the first purified terpene is Pulegone.

In one embodiment, the first purified cannabinoid is THCV and the first purified terpene is Camphene.

In one embodiment, the first purified cannabinoid is THCV and the first purified terpene is Geraniol.

In one embodiment, the first purified cannabinoid is THCVA and the first purified terpene is Limonene.

In one embodiment, the first purified cannabinoid is THCVA and the first purified terpene is Nerolidol.

In one embodiment, the first purified cannabinoid is THCVA and the first purified terpene is Beta-Myrcene.

In one embodiment, the first purified cannabinoid is THCVA and the first purified terpene is Linalool.

In one embodiment, the first purified cannabinoid is THCVA and the first purified terpene is Alpha-Caryophyllene.

In one embodiment, the first purified cannabinoid is THCVA and the first purified terpene is Beta-Caryophyllene.

In one embodiment, the first purified cannabinoid is THCVA and the first purified terpene is Alpha-Pinene.

In one embodiment, the first purified cannabinoid is THCVA and the first purified terpene is Beta-Pinene.

In one embodiment, the first purified cannabinoid is THCVA and the first purified terpene is Alpha-Bisabolol.

In one embodiment, the first purified cannabinoid is THCVA and the first purified terpene is Delta-3-Carene.

In one embodiment, the first purified cannabinoid is THCVA and the first purified terpene is Borneol.

In one embodiment, the first purified cannabinoid is THCVA and the first purified terpene is Eucalyptol.

In one embodiment, the first purified cannabinoid is THCVA and the first purified terpene is p-Cymene.

In one embodiment, the first purified cannabinoid is THCVA and the first purified terpene is Alpha-Terpineol.

In one embodiment, the first purified cannabinoid is THCVA and the first purified terpene is Alpha-Humulene.

In one embodiment, the first purified cannabinoid is THCVA and the first purified terpene is Terpinolene.

In one embodiment, the first purified cannabinoid is THCVA and the first purified terpene is Pulegone.

In one embodiment, the first purified cannabinoid is THCVA and the first purified terpene is Camphene.

In one embodiment, the first purified cannabinoid is THCVA and the first purified terpene is Geraniol.

In one embodiment, the first purified cannabinoid is CBD and the first purified terpene is Limonene.

In one embodiment, the first purified cannabinoid is CBD and the first purified terpene is Nerolidol.

In one embodiment, the first purified cannabinoid is CBD and the first purified terpene is Beta-Myrcene.

In one embodiment, the first purified cannabinoid is CBD and the first purified terpene is Linalool.

In one embodiment, the first purified cannabinoid is CBD and the first purified terpene is Alpha-Caryophyllene.

In one embodiment, the first purified cannabinoid is CBD and the first purified terpene is Beta-Caryophyllene.

In one embodiment, the first purified cannabinoid is CBD and the first purified terpene is Alpha-Pinene.

In one embodiment, the first purified cannabinoid is CBD and the first purified terpene is Beta-Pinene.

In one embodiment, the first purified cannabinoid is CBD and the first purified terpene is Alpha-Bisabolol.

In one embodiment, the first purified cannabinoid is CBD and the first purified terpene is Delta-3-Carene.

In one embodiment, the first purified cannabinoid is CBD and the first purified terpene is Borneol.

In one embodiment, the first purified cannabinoid is CBD and the first purified terpene is Eucalyptol.

In one embodiment, the first purified cannabinoid is CBD and the first purified terpene is p-Cymene.

In one embodiment, the first purified cannabinoid is CBD and the first purified terpene is Alpha-Terpineol.

In one embodiment, the first purified cannabinoid is CBD and the first purified terpene is Alpha-Humulene.

In one embodiment, the first purified cannabinoid is CBD and the first purified terpene is Terpinolene.

In one embodiment, the first purified cannabinoid is CBD and the first purified terpene is Pulegone.

In one embodiment, the first purified cannabinoid is CBD and the first purified terpene is Camphene.

In one embodiment, the first purified cannabinoid is CBD and the first purified terpene is Geraniol.

In one embodiment, the first purified cannabinoid is CBDA and the first purified terpene is Limonene.

In one embodiment, the first purified cannabinoid is CBDA and the first purified terpene is Nerolidol.

In one embodiment, the first purified cannabinoid is CBDA and the first purified terpene is Beta-Myrcene.

In one embodiment, the first purified cannabinoid is CBDA and the first purified terpene is Linalool.

In one embodiment, the first purified cannabinoid is CBDA and the first purified terpene is Alpha-Caryophyllene.

In one embodiment, the first purified cannabinoid is CBDA and the first purified terpene is Beta-Caryophyllene.

In one embodiment, the first purified cannabinoid is CBDA and the first purified terpene is Alpha-Pinene.

In one embodiment, the first purified cannabinoid is CBDA and the first purified terpene is Beta-Pinene.

In one embodiment, the first purified cannabinoid is CBDA and the first purified terpene is Alpha-Bisabolol.

In one embodiment, the first purified cannabinoid is CBDA and the first purified terpene is Delta-3-Carene.

In one embodiment, the first purified cannabinoid is CBDA and the first purified terpene is Borneol.

In one embodiment, the first purified cannabinoid is CBDA and the first purified terpene is Eucalyptol.

In one embodiment, the first purified cannabinoid is CBDA and the first purified terpene is p-Cymene.

In one embodiment, the first purified cannabinoid is CBDA and the first purified terpene is Alpha-Terpineol.

In one embodiment, the first purified cannabinoid is CBDA and the first purified terpene is Alpha-Humulene.

In one embodiment, the first purified cannabinoid is CBDA and the first purified terpene is Terpinolene.

In one embodiment, the first purified cannabinoid is CBDA and the first purified terpene is Pulegone.

In one embodiment, the first purified cannabinoid is CBDA and the first purified terpene is Camphene.

In one embodiment, the first purified cannabinoid is CBDA and the first purified terpene is Geraniol.

In one embodiment, the first purified cannabinoid is CBDV and the first purified terpene is Limonene.

In one embodiment, the first purified cannabinoid is CBDV and the first purified terpene is Nerolidol.

In one embodiment, the first purified cannabinoid is CBDV and the first purified terpene is Beta-Myrcene.

In one embodiment, the first purified cannabinoid is CBDV and the first purified terpene is Linalool.

In one embodiment, the first purified cannabinoid is CBDV and the first purified terpene is Alpha-Caryophyllene.

In one embodiment, the first purified cannabinoid is CBDV and the first purified terpene is Beta-Caryophyllene.

In one embodiment, the first purified cannabinoid is CBDV and the first purified terpene is Alpha-Pinene.

In one embodiment, the first purified cannabinoid is CBDV and the first purified terpene is Beta-Pinene.

In one embodiment, the first purified cannabinoid is CBDV and the first purified terpene is Alpha-Bisabolol.

In one embodiment, the first purified cannabinoid is CBDV and the first purified terpene is Delta-3-Carene.

In one embodiment, the first purified cannabinoid is CBDV and the first purified terpene is Borneol.

In one embodiment, the first purified cannabinoid is CBDV and the first purified terpene is Eucalyptol.

In one embodiment, the first purified cannabinoid is CBDV and the first purified terpene is p-Cymene.

In one embodiment, the first purified cannabinoid is CBDV and the first purified terpene is Alpha-Terpineol.

In one embodiment, the first purified cannabinoid is CBDV and the first purified terpene is Alpha-Humulene.

In one embodiment, the first purified cannabinoid is CBDV and the first purified terpene is Terpinolene.

In one embodiment, the first purified cannabinoid is CBDV and the first purified terpene is Pulegone.

In one embodiment, the first purified cannabinoid is CBDV and the first purified terpene is Camphene.

In one embodiment, the first purified cannabinoid is CBDV and the first purified terpene is Geraniol.

In one embodiment, the first purified cannabinoid is CBDVA and the first purified terpene is Limonene.

In one embodiment, the first purified cannabinoid is CBDVA and the first purified terpene is Nerolidol.

In one embodiment, the first purified cannabinoid is CBDVA and the first purified terpene is Beta-Myrcene.

In one embodiment, the first purified cannabinoid is CBDVA and the first purified terpene is Linalool.

In one embodiment, the first purified cannabinoid is CBDVA and the first purified terpene is Alpha-Caryophyllene.

In one embodiment, the first purified cannabinoid is CBDVA and the first purified terpene is Beta-Caryophyllene.

In one embodiment, the first purified cannabinoid is CBDVA and the first purified terpene is Alpha-Pinene.

In one embodiment, the first purified cannabinoid is CBDVA and the first purified terpene is Beta-Pinene.

In one embodiment, the first purified cannabinoid is CBDVA and the first purified terpene is Alpha-Bisabolol.

In one embodiment, the first purified cannabinoid is CBDVA and the first purified terpene is Delta-3-Carene.

In one embodiment, the first purified cannabinoid is CBDVA and the first purified terpene is Borneol.

In one embodiment, the first purified cannabinoid is CBDVA and the first purified terpene is Eucalyptol.

In one embodiment, the first purified cannabinoid is CBDVA and the first purified terpene is p-Cymene.

In one embodiment, the first purified cannabinoid is CBDVA and the first purified terpene is Alpha-Terpineol.

In one embodiment, the first purified cannabinoid is CBDVA and the first purified terpene is Alpha-Humulene.

In one embodiment, the first purified cannabinoid is CBDVA and the first purified terpene is Terpinolene.

In one embodiment, the first purified cannabinoid is CBDVA and the first purified terpene is Pulegone.

In one embodiment, the first purified cannabinoid is CBDVA and the first purified terpene is Camphene.

In one embodiment, the first purified cannabinoid is CBDVA and the first purified terpene is Geraniol.

In one embodiment, the first purified cannabinoid is CBC and the first purified terpene is Limonene.

In one embodiment, the first purified cannabinoid is CBC and the first purified terpene is Nerolidol.

In one embodiment, the first purified cannabinoid is CBC and the first purified terpene is Beta-Myrcene.

In one embodiment, the first purified cannabinoid is CBC and the first purified terpene is Linalool.

In one embodiment, the first purified cannabinoid is CBC and the first purified terpene is Alpha-Caryophyllene.

In one embodiment, the first purified cannabinoid is CBC and the first purified terpene is Beta-Caryophyllene.

In one embodiment, the first purified cannabinoid is CBC and the first purified terpene is Alpha-Pinene.

In one embodiment, the first purified cannabinoid is CBC and the first purified terpene is Beta-Pinene.

In one embodiment, the first purified cannabinoid is CBC and the first purified terpene is Alpha-Bisabolol.

In one embodiment, the first purified cannabinoid is CBC and the first purified terpene is Delta-3-Carene.

In one embodiment, the first purified cannabinoid is CBC and the first purified terpene is Borneol.

In one embodiment, the first purified cannabinoid is CBC and the first purified terpene is Eucalyptol.

In one embodiment, the first purified cannabinoid is CBC and the first purified terpene is p-Cymene.

In one embodiment, the first purified cannabinoid is CBC and the first purified terpene is Alpha-Terpineol.

In one embodiment, the first purified cannabinoid is CBC and the first purified terpene is Alpha-Humulene.

In one embodiment, the first purified cannabinoid is CBC and the first purified terpene is Terpinolene.

In one embodiment, the first purified cannabinoid is CBC and the first purified terpene is Pulegone.

In one embodiment, the first purified cannabinoid is CBC and the first purified terpene is Camphene.

In one embodiment, the first purified cannabinoid is CBC and the first purified terpene is Geraniol.

In one embodiment, the first purified cannabinoid is CBCA and the first purified terpene is Limonene.

In one embodiment, the first purified cannabinoid is CBCA and the first purified terpene is Nerolidol.

In one embodiment, the first purified cannabinoid is CBCA and the first purified terpene is Beta-Myrcene.

In one embodiment, the first purified cannabinoid is CBCA and the first purified terpene is Linalool.

In one embodiment, the first purified cannabinoid is CBCA and the first purified terpene is Alpha-Caryophyllene.

In one embodiment, the first purified cannabinoid is CBCA and the first purified terpene is Beta-Caryophyllene.

In one embodiment, the first purified cannabinoid is CBCA and the first purified terpene is Alpha-Pinene.

In one embodiment, the first purified cannabinoid is CBCA and the first purified terpene is Beta-Pinene.

In one embodiment, the first purified cannabinoid is CBCA and the first purified terpene is Alpha-Bisabolol.

In one embodiment, the first purified cannabinoid is CBCA and the first purified terpene is Delta-3-Carene.

In one embodiment, the first purified cannabinoid is CBCA and the first purified terpene is Borneol.

In one embodiment, the first purified cannabinoid is CBCA and the first purified terpene is Eucalyptol.

In one embodiment, the first purified cannabinoid is CBCA and the first purified terpene is p-Cymene.

In one embodiment, the first purified cannabinoid is CBCA and the first purified terpene is Alpha-Terpineol.

In one embodiment, the first purified cannabinoid is CBCA and the first purified terpene is Alpha-Humulene.

In one embodiment, the first purified cannabinoid is CBCA and the first purified terpene is Terpinolene.

In one embodiment, the first purified cannabinoid is CBCA and the first purified terpene is Pulegone.

In one embodiment, the first purified cannabinoid is CBCA and the first purified terpene is Camphene.

In one embodiment, the first purified cannabinoid is CBCA and the first purified terpene is Geraniol.

In one embodiment, the first purified cannabinoid is CBCV and the first purified terpene is Limonene.

In one embodiment, the first purified cannabinoid is CBCV and the first purified terpene is Nerolidol.

In one embodiment, the first purified cannabinoid is CBCV and the first purified terpene is Beta-Myrcene.

In one embodiment, the first purified cannabinoid is CBCV and the first purified terpene is Linalool.

In one embodiment, the first purified cannabinoid is CBCV and the first purified terpene is Alpha-Caryophyllene.

In one embodiment, the first purified cannabinoid is CBCV and the first purified terpene is Beta-Caryophyllene.

In one embodiment, the first purified cannabinoid is CBCV and the first purified terpene is Alpha-Pinene.

In one embodiment, the first purified cannabinoid is CBCV and the first purified terpene is Beta-Pinene.

In one embodiment, the first purified cannabinoid is CBCV and the first purified terpene is Alpha-Bisabolol.

In one embodiment, the first purified cannabinoid is CBCV and the first purified terpene is Delta-3-Carene.

In one embodiment, the first purified cannabinoid is CBCV and the first purified terpene is Borneol.

In one embodiment, the first purified cannabinoid is CBCV and the first purified terpene is Eucalyptol.

In one embodiment, the first purified cannabinoid is CBCV and the first purified terpene is p-Cymene.

In one embodiment, the first purified cannabinoid is CBCV and the first purified terpene is Alpha-Terpineol.

In one embodiment, the first purified cannabinoid is CBCV and the first purified terpene is Alpha-Humulene.

In one embodiment, the first purified cannabinoid is CBCV and the first purified terpene is Terpinolene.

In one embodiment, the first purified cannabinoid is CBCV and the first purified terpene is Pulegone.

In one embodiment, the first purified cannabinoid is CBCV and the first purified terpene is Camphene.

In one embodiment, the first purified cannabinoid is CBCV and the first purified terpene is Geraniol.

In one embodiment, the first purified cannabinoid is CBCVA and the first purified terpene is Limonene.

In one embodiment, the first purified cannabinoid is CBCVA and the first purified terpene is Nerolidol.

In one embodiment, the first purified cannabinoid is CBCVA and the first purified terpene is Beta-Myrcene.

In one embodiment, the first purified cannabinoid is CBCVA and the first purified terpene is Linalool.

In one embodiment, the first purified cannabinoid is CBCVA and the first purified terpene is Alpha-Caryophyllene.

In one embodiment, the first purified cannabinoid is CBCVA and the first purified terpene is Beta-Caryophyllene.

In one embodiment, the first purified cannabinoid is CBCVA and the first purified terpene is Alpha-Pinene.

In one embodiment, the first purified cannabinoid is CBCVA and the first purified terpene is Beta-Pinene.

In one embodiment, the first purified cannabinoid is CBCVA and the first purified terpene is Alpha-Bisabolol.

In one embodiment, the first purified cannabinoid is CBCVA and the first purified terpene is Delta-3-Carene.

In one embodiment, the first purified cannabinoid is CBCVA and the first purified terpene is Borneol.

In one embodiment, the first purified cannabinoid is CBCVA and the first purified terpene is Eucalyptol.

In one embodiment, the first purified cannabinoid is CBCVA and the first purified terpene is p-Cymene.

In one embodiment, the first purified cannabinoid is CBCVA and the first purified terpene is Alpha-Terpineol.

In one embodiment, the first purified cannabinoid is CBCVA and the first purified terpene is Alpha-Humulene.

In one embodiment, the first purified cannabinoid is CBCVA and the first purified terpene is Terpinolene.

In one embodiment, the first purified cannabinoid is CBCVA and the first purified terpene is Pulegone.

In one embodiment, the first purified cannabinoid is CBCVA and the first purified terpene is Camphene.

In one embodiment, the first purified cannabinoid is CBCVA and the first purified terpene is Geraniol.

In one embodiment, the first purified cannabinoid is CBG and the first purified terpene is Limonene.

In one embodiment, the first purified cannabinoid is CBG and the first purified terpene is Nerolidol.

In one embodiment, the first purified cannabinoid is CBG and the first purified terpene is Beta-Myrcene.

In one embodiment, the first purified cannabinoid is CBG and the first purified terpene is Linalool.

In one embodiment, the first purified cannabinoid is CBG and the first purified terpene is Alpha-Caryophyllene.

In one embodiment, the first purified cannabinoid is CBG and the first purified terpene is Beta-Caryophyllene.

In one embodiment, the first purified cannabinoid is CBG and the first purified terpene is Alpha-Pinene.

In one embodiment, the first purified cannabinoid is CBG and the first purified terpene is Beta-Pinene.

In one embodiment, the first purified cannabinoid is CBG and the first purified terpene is Alpha-Bisabolol.

In one embodiment, the first purified cannabinoid is CBG and the first purified terpene is Delta-3-Carene.

In one embodiment, the first purified cannabinoid is CBG and the first purified terpene is Borneol.

In one embodiment, the first purified cannabinoid is CBG and the first purified terpene is Eucalyptol.

In one embodiment, the first purified cannabinoid is CBG and the first purified terpene is p-Cymene.

In one embodiment, the first purified cannabinoid is CBG and the first purified terpene is Alpha-Terpineol.

In one embodiment, the first purified cannabinoid is CBG and the first purified terpene is Alpha-Humulene.

In one embodiment, the first purified cannabinoid is CBG and the first purified terpene is Terpinolene.

In one embodiment, the first purified cannabinoid is CBG and the first purified terpene is Pulegone.

In one embodiment, the first purified cannabinoid is CBG and the first purified terpene is Camphene.

In one embodiment, the first purified cannabinoid is CBG and the first purified terpene is Geraniol.

In one embodiment, the first purified cannabinoid is CBGA and the first purified terpene is Limonene.

In one embodiment, the first purified cannabinoid is CBGA and the first purified terpene is Nerolidol.

In one embodiment, the first purified cannabinoid is CBGA and the first purified terpene is Beta-Myrcene.

In one embodiment, the first purified cannabinoid is CBGA and the first purified terpene is Linalool.

In one embodiment, the first purified cannabinoid is CBGA and the first purified terpene is Alpha-Caryophyllene.

In one embodiment, the first purified cannabinoid is CBGA and the first purified terpene is Beta-Caryophyllene.

In one embodiment, the first purified cannabinoid is CBGA and the first purified terpene is Alpha-Pinene.

In one embodiment, the first purified cannabinoid is CBGA and the first purified terpene is Beta-Pinene.

In one embodiment, the first purified cannabinoid is CBGA and the first purified terpene is Alpha-Bisabolol.

In one embodiment, the first purified cannabinoid is CBGA and the first purified terpene is Delta-3-Carene.

In one embodiment, the first purified cannabinoid is CBGA and the first purified terpene is Borneol.

In one embodiment, the first purified cannabinoid is CBGA and the first purified terpene is Eucalyptol.

In one embodiment, the first purified cannabinoid is CBGA and the first purified terpene is p-Cymene.

In one embodiment, the first purified cannabinoid is CBGA and the first purified terpene is Alpha-Terpineol.

In one embodiment, the first purified cannabinoid is CBGA and the first purified terpene is Alpha-Humulene.

In one embodiment, the first purified cannabinoid is CBGA and the first purified terpene is Terpinolene.

In one embodiment, the first purified cannabinoid is CBGA and the first purified terpene is Pulegone.

In one embodiment, the first purified cannabinoid is CBGA and the first purified terpene is Camphene.

In one embodiment, the first purified cannabinoid is CBGA and the first purified terpene is Geraniol.

In one embodiment, the first purified cannabinoid is CBGV and the first purified terpene is Limonene.

In one embodiment, the first purified cannabinoid is CBGV and the first purified terpene is Nerolidol.

In one embodiment, the first purified cannabinoid is CBGV and the first purified terpene is Beta-Myrcene.

In one embodiment, the first purified cannabinoid is CBGV and the first purified terpene is Linalool.

In one embodiment, the first purified cannabinoid is CBGV and the first purified terpene is Alpha-Caryophyllene.

In one embodiment, the first purified cannabinoid is CBGV and the first purified terpene is Beta-Caryophyllene.

In one embodiment, the first purified cannabinoid is CBGV and the first purified terpene is Alpha-Pinene.

In one embodiment, the first purified cannabinoid is CBGV and the first purified terpene is Beta-Pinene.

In one embodiment, the first purified cannabinoid is CBGV and the first purified terpene is Alpha-Bisabolol.

In one embodiment, the first purified cannabinoid is CBGV and the first purified terpene is Delta-3-Carene.

In one embodiment, the first purified cannabinoid is CBGV and the first purified terpene is Borneol.

In one embodiment, the first purified cannabinoid is CBGV and the first purified terpene is Eucalyptol.

In one embodiment, the first purified cannabinoid is CBGV and the first purified terpene is p-Cymene.

In one embodiment, the first purified cannabinoid is CBGV and the first purified terpene is Alpha-Terpineol.

In one embodiment, the first purified cannabinoid is CBGV and the first purified terpene is Alpha-Humulene.

In one embodiment, the first purified cannabinoid is CBGV and the first purified terpene is Terpinolene.

In one embodiment, the first purified cannabinoid is CBGV and the first purified terpene is Pulegone.

In one embodiment, the first purified cannabinoid is CBGV and the first purified terpene is Camphene.

In one embodiment, the first purified cannabinoid is CBGV and the first purified terpene is Geraniol.

In one embodiment, the first purified cannabinoid is CBGVA and the first purified terpene is Limonene.

In one embodiment, the first purified cannabinoid is CBGVA and the first purified terpene is Nerolidol.

In one embodiment, the first purified cannabinoid is CBGVA and the first purified terpene is Beta-Myrcene.

In one embodiment, the first purified cannabinoid is CBGVA and the first purified terpene is Linalool.

In one embodiment, the first purified cannabinoid is CBGVA and the first purified terpene is Alpha-Caryophyllene.

In one embodiment, the first purified cannabinoid is CBGVA and the first purified terpene is Beta-Caryophyllene.

In one embodiment, the first purified cannabinoid is CBGVA and the first purified terpene is Alpha-Pinene.

In one embodiment, the first purified cannabinoid is CBGVA and the first purified terpene is Beta-Pinene.

In one embodiment, the first purified cannabinoid is CBGVA and the first purified terpene is Alpha-Bisabolol.

In one embodiment, the first purified cannabinoid is CBGVA and the first purified terpene is Delta-3-Carene.

In one embodiment, the first purified cannabinoid is CBGVA and the first purified terpene is Borneol.

In one embodiment, the first purified cannabinoid is CBGVA and the first purified terpene is Eucalyptol.

In one embodiment, the first purified cannabinoid is CBGVA and the first purified terpene is p-Cymene.

In one embodiment, the first purified cannabinoid is CBGVA and the first purified terpene is Alpha-Terpineol.

In one embodiment, the first purified cannabinoid is CBGVA and the first purified terpene is Alpha-Humulene.

In one embodiment, the first purified cannabinoid is CBGVA and the first purified terpene is Terpinolene.

In one embodiment, the first purified cannabinoid is CBGVA and the first purified terpene is Pulegone.

In one embodiment, the first purified cannabinoid is CBGVA and the first purified terpene is Camphene.

In one embodiment, the first purified cannabinoid is CBGVA and the first purified terpene is Geraniol.

In one embodiment, the first purified cannabinoid is CBN and the first purified terpene is Limonene.

In one embodiment, the first purified cannabinoid is CBN and the first purified terpene is Nerolidol.

In one embodiment, the first purified cannabinoid is CBN and the first purified terpene is Beta-Myrcene.

In one embodiment, the first purified cannabinoid is CBN and the first purified terpene is Linalool.

In one embodiment, the first purified cannabinoid is CBN and the first purified terpene is Alpha-Caryophyllene.

In one embodiment, the first purified cannabinoid is CBN and the first purified terpene is Beta-Caryophyllene.

In one embodiment, the first purified cannabinoid is CBN and the first purified terpene is Alpha-Pinene.

In one embodiment, the first purified cannabinoid is CBN and the first purified terpene is Beta-Pinene.

In one embodiment, the first purified cannabinoid is CBN and the first purified terpene is Alpha-Bisabolol.

In one embodiment, the first purified cannabinoid is CBN and the first purified terpene is Delta-3-Carene.

In one embodiment, the first purified cannabinoid is CBN and the first purified terpene is Borneol.

In one embodiment, the first purified cannabinoid is CBN and the first purified terpene is Eucalyptol.

In one embodiment, the first purified cannabinoid is CBN and the first purified terpene is p-Cymene.

In one embodiment, the first purified cannabinoid is CBN and the first purified terpene is Alpha-Terpineol.

In one embodiment, the first purified cannabinoid is CBN and the first purified terpene is Alpha-Humulene.

In one embodiment, the first purified cannabinoid is CBN and the first purified terpene is Terpinolene.

In one embodiment, the first purified cannabinoid is CBN and the first purified terpene is Pulegone.

In one embodiment, the first purified cannabinoid is CBN and the first purified terpene is Camphene.

In one embodiment, the first purified cannabinoid is CBN and the first purified terpene is Geraniol.

In one embodiment, the first purified cannabinoid is CBNA and the first purified terpene is Limonene.

In one embodiment, the first purified cannabinoid is CBNA and the first purified terpene is Nerolidol.

In one embodiment, the first purified cannabinoid is CBNA and the first purified terpene is Beta-Myrcene.

In one embodiment, the first purified cannabinoid is CBNA and the first purified terpene is Linalool.

In one embodiment, the first purified cannabinoid is CBNA and the first purified terpene is Alpha-Caryophyllene.

In one embodiment, the first purified cannabinoid is CBNA and the first purified terpene is Beta-Caryophyllene.

In one embodiment, the first purified cannabinoid is CBNA and the first purified terpene is Alpha-Pinene.

In one embodiment, the first purified cannabinoid is CBNA and the first purified terpene is Beta-Pinene.

In one embodiment, the first purified cannabinoid is CBNA and the first purified terpene is Alpha-Bisabolol.

In one embodiment, the first purified cannabinoid is CBNA and the first purified terpene is Delta-3-Carene.

In one embodiment, the first purified cannabinoid is CBNA and the first purified terpene is Borneol.

In one embodiment, the first purified cannabinoid is CBNA and the first purified terpene is Eucalyptol.

In one embodiment, the first purified cannabinoid is CBNA and the first purified terpene is p-Cymene.

In one embodiment, the first purified cannabinoid is CBNA and the first purified terpene is Alpha-Terpineol.

In one embodiment, the first purified cannabinoid is CBNA and the first purified terpene is Alpha-Humulene.

In one embodiment, the first purified cannabinoid is CBNA and the first purified terpene is Terpinolene.

In one embodiment, the first purified cannabinoid is CBNA and the first purified terpene is Pulegone.

In one embodiment, the first purified cannabinoid is CBNA and the first purified terpene is Camphene.

In one embodiment, the first purified cannabinoid is CBNA and the first purified terpene is Geraniol.

In one embodiment, the first purified cannabinoid is CBNV and the first purified terpene is Limonene.

In one embodiment, the first purified cannabinoid is CBNV and the first purified terpene is Nerolidol.

In one embodiment, the first purified cannabinoid is CBNV and the first purified terpene is Beta-Myrcene.

In one embodiment, the first purified cannabinoid is CBNV and the first purified terpene is Linalool.

In one embodiment, the first purified cannabinoid is CBNV and the first purified terpene is Alpha-Caryophyllene.

In one embodiment, the first purified cannabinoid is CBNV and the first purified terpene is Beta-Caryophyllene.

In one embodiment, the first purified cannabinoid is CBNV and the first purified terpene is Alpha-Pinene.

In one embodiment, the first purified cannabinoid is CBNV and the first purified terpene is Beta-Pinene.

In one embodiment, the first purified cannabinoid is CBNV and the first purified terpene is Alpha-Bisabolol.

In one embodiment, the first purified cannabinoid is CBNV and the first purified terpene is Delta-3-Carene.

In one embodiment, the first purified cannabinoid is CBNV and the first purified terpene is Borneol.

In one embodiment, the first purified cannabinoid is CBNV and the first purified terpene is Eucalyptol.

In one embodiment, the first purified cannabinoid is CBNV and the first purified terpene is p-Cymene.

In one embodiment, the first purified cannabinoid is CBNV and the first purified terpene is Alpha-Terpineol.

In one embodiment, the first purified cannabinoid is CBNV and the first purified terpene is Alpha-Humulene.

In one embodiment, the first purified cannabinoid is CBNV and the first purified terpene is Terpinolene.

In one embodiment, the first purified cannabinoid is CBNV and the first purified terpene is Pulegone.

In one embodiment, the first purified cannabinoid is CBNV and the first purified terpene is Camphene.

In one embodiment, the first purified cannabinoid is CBNV and the first purified terpene is Geraniol.

In one embodiment, the first purified cannabinoid is CBNVA and the first purified terpene is Limonene.

In one embodiment, the first purified cannabinoid is CBNVA and the first purified terpene is Nerolidol.

In one embodiment, the first purified cannabinoid is CBNVA and the first purified terpene is Beta-Myrcene.

In one embodiment, the first purified cannabinoid is CBNVA and the first purified terpene is Linalool.

In one embodiment, the first purified cannabinoid is CBNVA and the first purified terpene is Alpha-Caryophyllene.

In one embodiment, the first purified cannabinoid is CBNVA and the first purified terpene is Beta-Caryophyllene.

In one embodiment, the first purified cannabinoid is CBNVA and the first purified terpene is Alpha-Pinene.

In one embodiment, the first purified cannabinoid is CBNVA and the first purified terpene is Beta-Pinene.

In one embodiment, the first purified cannabinoid is CBNVA and the first purified terpene is Alpha-Bisabolol.

In one embodiment, the first purified cannabinoid is CBNVA and the first purified terpene is Delta-3-Carene.

In one embodiment, the first purified cannabinoid is CBNVA and the first purified terpene is Borneol.

In one embodiment, the first purified cannabinoid is CBNVA and the first purified terpene is Eucalyptol.

In one embodiment, the first purified cannabinoid is CBNVA and the first purified terpene is p-Cymene.

In one embodiment, the first purified cannabinoid is CBNVA and the first purified terpene is Alpha-Terpineol.

In one embodiment, the first purified cannabinoid is CBNVA and the first purified terpene is Alpha-Humulene.

In one embodiment, the first purified cannabinoid is CBNVA and the first purified terpene is Terpinolene.

In one embodiment, the first purified cannabinoid is CBNVA and the first purified terpene is Pulegone.

In one embodiment, the first purified cannabinoid is CBNVA and the first purified terpene is Camphene.

In one embodiment, the first purified cannabinoid is CBNVA and the first purified terpene is Geraniol.

In one embodiment, the first purified cannabinoid is CBND and the first purified terpene is Limonene.

In one embodiment, the first purified cannabinoid is CBND and the first purified terpene is Nerolidol.

In one embodiment, the first purified cannabinoid is CBND and the first purified terpene is Beta-Myrcene.

In one embodiment, the first purified cannabinoid is CBND and the first purified terpene is Linalool.

In one embodiment, the first purified cannabinoid is CBND and the first purified terpene is Alpha-Caryophyllene.

In one embodiment, the first purified cannabinoid is CBND and the first purified terpene is Beta-Caryophyllene.

In one embodiment, the first purified cannabinoid is CBND and the first purified terpene is Alpha-Pinene.

In one embodiment, the first purified cannabinoid is CBND and the first purified terpene is Beta-Pinene.

In one embodiment, the first purified cannabinoid is CBND and the first purified terpene is Alpha-Bisabolol.

In one embodiment, the first purified cannabinoid is CBND and the first purified terpene is Delta-3-Carene.

In one embodiment, the first purified cannabinoid is CBND and the first purified terpene is Borneol.

In one embodiment, the first purified cannabinoid is CBND and the first purified terpene is Eucalyptol.

In one embodiment, the first purified cannabinoid is CBND and the first purified terpene is p-Cymene.

In one embodiment, the first purified cannabinoid is CBND and the first purified terpene is Alpha-Terpineol.

In one embodiment, the first purified cannabinoid is CBND and the first purified terpene is Alpha-Humulene.

In one embodiment, the first purified cannabinoid is CBND and the first purified terpene is Terpinolene.

In one embodiment, the first purified cannabinoid is CBND and the first purified terpene is Pulegone.

In one embodiment, the first purified cannabinoid is CBND and the first purified terpene is Camphene.

In one embodiment, the first purified cannabinoid is CBND and the first purified terpene is Geraniol.

In one embodiment, the first purified cannabinoid is CBNDA and the first purified terpene is Limonene.

In one embodiment, the first purified cannabinoid is CBNDA and the first purified terpene is Nerolidol.

In one embodiment, the first purified cannabinoid is CBNDA and the first purified terpene is Beta-Myrcene.

In one embodiment, the first purified cannabinoid is CBNDA and the first purified terpene is Linalool.

In one embodiment, the first purified cannabinoid is CBNDA and the first purified terpene is Alpha-Caryophyllene.

In one embodiment, the first purified cannabinoid is CBNDA and the first purified terpene is Beta-Caryophyllene.

In one embodiment, the first purified cannabinoid is CBNDA and the first purified terpene is Alpha-Pinene.

In one embodiment, the first purified cannabinoid is CBNDA and the first purified terpene is Beta-Pinene.

In one embodiment, the first purified cannabinoid is CBNDA and the first purified terpene is Alpha-Bisabolol.

In one embodiment, the first purified cannabinoid is CBNDA and the first purified terpene is Delta-3-Carene.

In one embodiment, the first purified cannabinoid is CBNDA and the first purified terpene is Borneol.

In one embodiment, the first purified cannabinoid is CBNDA and the first purified terpene is Eucalyptol.

In one embodiment, the first purified cannabinoid is CBNDA and the first purified terpene is p-Cymene.

In one embodiment, the first purified cannabinoid is CBNDA and the first purified terpene is Alpha-Terpineol.

In one embodiment, the first purified cannabinoid is CBNDA and the first purified terpene is Alpha-Humulene.

In one embodiment, the first purified cannabinoid is CBNDA and the first purified terpene is Terpinolene.

In one embodiment, the first purified cannabinoid is CBNDA and the first purified terpene is Pulegone.

In one embodiment, the first purified cannabinoid is CBNDA and the first purified terpene is Camphene.

In one embodiment, the first purified cannabinoid is CBNDA and the first purified terpene is Geraniol.

In one embodiment, the first purified cannabinoid is CBNDV and the first purified terpene is Limonene.

In one embodiment, the first purified cannabinoid is CBNDV and the first purified terpene is Nerolidol.

In one embodiment, the first purified cannabinoid is CBNDV and the first purified terpene is Beta-Myrcene.

In one embodiment, the first purified cannabinoid is CBNDV and the first purified terpene is Linalool.

In one embodiment, the first purified cannabinoid is CBNDV and the first purified terpene is Alpha-Caryophyllene.

In one embodiment, the first purified cannabinoid is CBNDV and the first purified terpene is Beta-Caryophyllene.

In one embodiment, the first purified cannabinoid is CBNDV and the first purified terpene is Alpha-Pinene.

In one embodiment, the first purified cannabinoid is CBNDV and the first purified terpene is Beta-Pinene.

In one embodiment, the first purified cannabinoid is CBNDV and the first purified terpene is Alpha-Bisabolol.

In one embodiment, the first purified cannabinoid is CBNDV and the first purified terpene is Delta-3-Carene.

In one embodiment, the first purified cannabinoid is CBNDV and the first purified terpene is Borneol.

In one embodiment, the first purified cannabinoid is CBNDV and the first purified terpene is Eucalyptol.

In one embodiment, the first purified cannabinoid is CBNDV and the first purified terpene is p-Cymene.

In one embodiment, the first purified cannabinoid is CBNDV and the first purified terpene is Alpha-Terpineol.

In one embodiment, the first purified cannabinoid is CBNDV and the first purified terpene is Alpha-Humulene.

In one embodiment, the first purified cannabinoid is CBNDV and the first purified terpene is Terpinolene.

In one embodiment, the first purified cannabinoid is CBNDV and the first purified terpene is Pulegone.

In one embodiment, the first purified cannabinoid is CBNDV and the first purified terpene is Camphene.

In one embodiment, the first purified cannabinoid is CBNDV and the first purified terpene is Geraniol.

In one embodiment, the first purified cannabinoid is CBNDVA and the first purified terpene is Limonene.

In one embodiment, the first purified cannabinoid is CBNDVA and the first purified terpene is Nerolidol.

In one embodiment, the first purified cannabinoid is CBNDVA and the first purified terpene is Beta-Myrcene.

In one embodiment, the first purified cannabinoid is CBNDVA and the first purified terpene is Linalool.

In one embodiment, the first purified cannabinoid is CBNDVA and the first purified terpene is Alpha-Caryophyllene.

In one embodiment, the first purified cannabinoid is CBNDVA and the first purified terpene is Beta-Caryophyllene.

In one embodiment, the first purified cannabinoid is CBNDVA and the first purified terpene is Alpha-Pinene.

In one embodiment, the first purified cannabinoid is CBNDVA and the first purified terpene is Beta-Pinene.

In one embodiment, the first purified cannabinoid is CBNDVA and the first purified terpene is Alpha-Bisabolol.

In one embodiment, the first purified cannabinoid is CBNDVA and the first purified terpene is Delta-3-Carene.

In one embodiment, the first purified cannabinoid is CBNDVA and the first purified terpene is Borneol.

In one embodiment, the first purified cannabinoid is CBNDVA and the first purified terpene is Eucalyptol.

In one embodiment, the first purified cannabinoid is CBNDVA and the first purified terpene is p-Cymene.

In one embodiment, the first purified cannabinoid is CBNDVA and the first purified terpene is Alpha-Terpineol.

In one embodiment, the first purified cannabinoid is CBNDVA and the first purified terpene is Alpha-Humulene.

In one embodiment, the first purified cannabinoid is CBNDVA and the first purified terpene is Terpinolene.

In one embodiment, the first purified cannabinoid is CBNDVA and the first purified terpene is Pulegone.

In one embodiment, the first purified cannabinoid is CBNDVA and the first purified terpene is Camphene.

In one embodiment, the first purified cannabinoid is CBNDVA and the first purified terpene is Geraniol.

In one embodiment, the first purified cannabinoid is CBL and the first purified terpene is Limonene.

In one embodiment, the first purified cannabinoid is CBL and the first purified terpene is Nerolidol.

In one embodiment, the first purified cannabinoid is CBL and the first purified terpene is Beta-Myrcene.

In one embodiment, the first purified cannabinoid is CBL and the first purified terpene is Linalool.

In one embodiment, the first purified cannabinoid is CBL and the first purified terpene is Alpha-Caryophyllene.

In one embodiment, the first purified cannabinoid is CBL and the first purified terpene is Beta-Caryophyllene.

In one embodiment, the first purified cannabinoid is CBL and the first purified terpene is Alpha-Pinene.

In one embodiment, the first purified cannabinoid is CBL and the first purified terpene is Beta-Pinene.

In one embodiment, the first purified cannabinoid is CBL and the first purified terpene is Alpha-Bisabolol.

In one embodiment, the first purified cannabinoid is CBL and the first purified terpene is Delta-3-Carene.

In one embodiment, the first purified cannabinoid is CBL and the first purified terpene is Borneol.

In one embodiment, the first purified cannabinoid is CBL and the first purified terpene is Eucalyptol.

In one embodiment, the first purified cannabinoid is CBL and the first purified terpene is p-Cymene.

In one embodiment, the first purified cannabinoid is CBL and the first purified terpene is Alpha-Terpineol.

In one embodiment, the first purified cannabinoid is CBL and the first purified terpene is Alpha-Humulene.

In one embodiment, the first purified cannabinoid is CBL and the first purified terpene is Terpinolene.

In one embodiment, the first purified cannabinoid is CBL and the first purified terpene is Pulegone.

In one embodiment, the first purified cannabinoid is CBL and the first purified terpene is Camphene.

In one embodiment, the first purified cannabinoid is CBL and the first purified terpene is Geraniol.

In one embodiment, the first purified cannabinoid is CBLA and the first purified terpene is Limonene.

In one embodiment, the first purified cannabinoid is CBLA and the first purified terpene is Nerolidol.

In one embodiment, the first purified cannabinoid is CBLA and the first purified terpene is Beta-Myrcene.

In one embodiment, the first purified cannabinoid is CBLA and the first purified terpene is Linalool.

In one embodiment, the first purified cannabinoid is CBLA and the first purified terpene is Alpha-Caryophyllene.

In one embodiment, the first purified cannabinoid is CBLA and the first purified terpene is Beta-Caryophyllene.

In one embodiment, the first purified cannabinoid is CBLA and the first purified terpene is Alpha-Pinene.

In one embodiment, the first purified cannabinoid is CBLA and the first purified terpene is Beta-Pinene.

In one embodiment, the first purified cannabinoid is CBLA and the first purified terpene is Alpha-Bisabolol.

In one embodiment, the first purified cannabinoid is CBLA and the first purified terpene is Delta-3-Carene.

In one embodiment, the first purified cannabinoid is CBLA and the first purified terpene is Borneol.

In one embodiment, the first purified cannabinoid is CBLA and the first purified terpene is Eucalyptol.

In one embodiment, the first purified cannabinoid is CBLA and the first purified terpene is p-Cymene.

In one embodiment, the first purified cannabinoid is CBLA and the first purified terpene is Alpha-Terpineol.

In one embodiment, the first purified cannabinoid is CBLA and the first purified terpene is Alpha-Humulene.

In one embodiment, the first purified cannabinoid is CBLA and the first purified terpene is Terpinolene.

In one embodiment, the first purified cannabinoid is CBLA and the first purified terpene is Pulegone.

In one embodiment, the first purified cannabinoid is CBLA and the first purified terpene is Camphene.

In one embodiment, the first purified cannabinoid is CBLA and the first purified terpene is Geraniol.

In one embodiment, the first purified cannabinoid is CBLV and the first purified terpene is Limonene.

In one embodiment, the first purified cannabinoid is CBLV and the first purified terpene is Nerolidol.

In one embodiment, the first purified cannabinoid is CBLV and the first purified terpene is Beta-Myrcene.

In one embodiment, the first purified cannabinoid is CBLV and the first purified terpene is Linalool.

In one embodiment, the first purified cannabinoid is CBLV and the first purified terpene is Alpha-Caryophyllene.

In one embodiment, the first purified cannabinoid is CBLV and the first purified terpene is Beta-Caryophyllene.

In one embodiment, the first purified cannabinoid is CBLV and the first purified terpene is Alpha-Pinene.

In one embodiment, the first purified cannabinoid is CBLV and the first purified terpene is Beta-Pinene.

In one embodiment, the first purified cannabinoid is CBLV and the first purified terpene is Alpha-Bisabolol.

In one embodiment, the first purified cannabinoid is CBLV and the first purified terpene is Delta-3-Carene.

In one embodiment, the first purified cannabinoid is CBLV and the first purified terpene is Borneol.

In one embodiment, the first purified cannabinoid is CBLV and the first purified terpene is Eucalyptol.

In one embodiment, the first purified cannabinoid is CBLV and the first purified terpene is p-Cymene.

In one embodiment, the first purified cannabinoid is CBLV and the first purified terpene is Alpha-Terpineol.

In one embodiment, the first purified cannabinoid is CBLV and the first purified terpene is Alpha-Humulene.

In one embodiment, the first purified cannabinoid is CBLV and the first purified terpene is Terpinolene.

In one embodiment, the first purified cannabinoid is CBLV and the first purified terpene is Pulegone.

In one embodiment, the first purified cannabinoid is CBLV and the first purified terpene is Camphene.

In one embodiment, the first purified cannabinoid is CBLV and the first purified terpene is Geraniol.

In one embodiment, the first purified cannabinoid is CBLVA and the first purified terpene is Limonene.

In one embodiment, the first purified cannabinoid is CBLVA and the first purified terpene is Nerolidol.

In one embodiment, the first purified cannabinoid is CBLVA and the first purified terpene is Beta-Myrcene.

In one embodiment, the first purified cannabinoid is CBLVA and the first purified terpene is Linalool.

In one embodiment, the first purified cannabinoid is CBLVA and the first purified terpene is Alpha-Caryophyllene.

In one embodiment, the first purified cannabinoid is CBLVA and the first purified terpene is Beta-Caryophyllene.

In one embodiment, the first purified cannabinoid is CBLVA and the first purified terpene is Alpha-Pinene.

In one embodiment, the first purified cannabinoid is CBLVA and the first purified terpene is Beta-Pinene.

In one embodiment, the first purified cannabinoid is CBLVA and the first purified terpene is Alpha-Bisabolol.

In one embodiment, the first purified cannabinoid is CBLVA and the first purified terpene is Delta-3-Carene.

In one embodiment, the first purified cannabinoid is CBLVA and the first purified terpene is Borneol.

In one embodiment, the first purified cannabinoid is CBLVA and the first purified terpene is Eucalyptol.

In one embodiment, the first purified cannabinoid is CBLVA and the first purified terpene is p-Cymene.

In one embodiment, the first purified cannabinoid is CBLVA and the first purified terpene is Alpha-Terpineol.

In one embodiment, the first purified cannabinoid is CBLVA and the first purified terpene is Alpha-Humulene.

In one embodiment, the first purified cannabinoid is CBLVA and the first purified terpene is Terpinolene.

In one embodiment, the first purified cannabinoid is CBLVA and the first purified terpene is Pulegone.

In one embodiment, the first purified cannabinoid is CBLVA and the first purified terpene is Camphene.

In one embodiment, the first purified cannabinoid is CBLVA and the first purified terpene is Geraniol.

In one embodiment, the first purified cannabinoid is CBE and the first purified terpene is Limonene.

In one embodiment, the first purified cannabinoid is CBE and the first purified terpene is Nerolidol

In one embodiment, the first purified cannabinoid is CBE and the first purified terpene is Beta-Myrcene.

In one embodiment, the first purified cannabinoid is CBE and the first purified terpene is Linalool.

In one embodiment, the first purified cannabinoid is CBE and the first purified terpene is Alpha-Caryophyllene.

In one embodiment, the first purified cannabinoid is CBE and the first purified terpene is Beta-Caryophyllene.

In one embodiment, the first purified cannabinoid is CBE and the first purified terpene is Alpha-Pinene.

In one embodiment, the first purified cannabinoid is CBE and the first purified terpene is Beta-Pinene.

In one embodiment, the first purified cannabinoid is CBE and the first purified terpene is Alpha-Bisabolol.

In one embodiment, the first purified cannabinoid is CBE and the first purified terpene is Delta-3-Carene.

In one embodiment, the first purified cannabinoid is CBE and the first purified terpene is Borneol.

In one embodiment, the first purified cannabinoid is CBE and the first purified terpene is Eucalyptol.

In one embodiment, the first purified cannabinoid is CBE and the first purified terpene is p-Cymene.

In one embodiment, the first purified cannabinoid is CBE and the first purified terpene is Alpha-Terpineol.

In one embodiment, the first purified cannabinoid is CBE and the first purified terpene is Alpha-Humulene.

In one embodiment, the first purified cannabinoid is CBE and the first purified terpene is Terpinolene.

In one embodiment, the first purified cannabinoid is CBE and the first purified terpene is Pulegone.

In one embodiment, the first purified cannabinoid is CBE and the first purified terpene is Camphene.

In one embodiment, the first purified cannabinoid is CBE and the first purified terpene is Geraniol.

In one embodiment, the first purified cannabinoid is CBEA and the first purified terpene is Limonene.

In one embodiment, the first purified cannabinoid is CBEA and the first purified terpene is Nerolidol

In one embodiment, the first purified cannabinoid is CBEA and the first purified terpene is Beta-Myrcene.

In one embodiment, the first purified cannabinoid is CBEA and the first purified terpene is Linalool.

In one embodiment, the first purified cannabinoid is CBEA and the first purified terpene is Alpha-Caryophyllene.

In one embodiment, the first purified cannabinoid is CBEA and the first purified terpene is Beta-Caryophyllene.

In one embodiment, the first purified cannabinoid is CBEA and the first purified terpene is Alpha-Pinene.

In one embodiment, the first purified cannabinoid is CBEA and the first purified terpene is Beta-Pinene.

In one embodiment, the first purified cannabinoid is CBEA and the first purified terpene is Alpha-Bisabolol.

In one embodiment, the first purified cannabinoid is CBEA and the first purified terpene is Delta-3-Carene.

In one embodiment, the first purified cannabinoid is CBEA and the first purified terpene is Borneol.

In one embodiment, the first purified cannabinoid is CBEA and the first purified terpene is Eucalyptol.

In one embodiment, the first purified cannabinoid is CBEA and the first purified terpene is p-Cymene.

In one embodiment, the first purified cannabinoid is CBEA and the first purified terpene is Alpha-Terpineol.

In one embodiment, the first purified cannabinoid is CBEA and the first purified terpene is Alpha-Humulene.

In one embodiment, the first purified cannabinoid is CBEA and the first purified terpene is Terpinolene.

In one embodiment, the first purified cannabinoid is CBEA and the first purified terpene is Pulegone.

In one embodiment, the first purified cannabinoid is CBEA and the first purified terpene is Camphene.

In one embodiment, the first purified cannabinoid is CBEA and the first purified terpene is Geraniol.

In one embodiment, the first purified cannabinoid is CBEV and the first purified terpene is Limonene.

In one embodiment, the first purified cannabinoid is CBEV and the first purified terpene is Nerolidol

In one embodiment, the first purified cannabinoid is CBEV and the first purified terpene is Beta-Myrcene.

In one embodiment, the first purified cannabinoid is CBEV and the first purified terpene is Linalool.

In one embodiment, the first purified cannabinoid is CBEV and the first purified terpene is Alpha-Caryophyllene.

In one embodiment, the first purified cannabinoid is CBEV and the first purified terpene is Beta-Caryophyllene.

In one embodiment, the first purified cannabinoid is CBEV and the first purified terpene is Alpha-Pinene.

In one embodiment, the first purified cannabinoid is CBEV and the first purified terpene is Beta-Pinene.

In one embodiment, the first purified cannabinoid is CBEV and the first purified terpene is Alpha-Bisabolol.

In one embodiment, the first purified cannabinoid is CBEV and the first purified terpene is Delta-3-Carene.

In one embodiment, the first purified cannabinoid is CBEV and the first purified terpene is Borneol.

In one embodiment, the first purified cannabinoid is CBEV and the first purified terpene is Eucalyptol.

In one embodiment, the first purified cannabinoid is CBEV and the first purified terpene is p-Cymene.

In one embodiment, the first purified cannabinoid is CBEV and the first purified terpene is Alpha-Terpineol.

In one embodiment, the first purified cannabinoid is CBEV and the first purified terpene is Alpha-Humulene.

In one embodiment, the first purified cannabinoid is CBEV and the first purified terpene is Terpinolene.

In one embodiment, the first purified cannabinoid is CBEV and the first purified terpene is Pulegone.

In one embodiment, the first purified cannabinoid is CBEV and the first purified terpene is Camphene.

In one embodiment, the first purified cannabinoid is CBEV and the first purified terpene is Geraniol.

In one embodiment, the first purified cannabinoid is CBEVA and the first purified terpene is Limonene.

In one embodiment, the first purified cannabinoid is CBEVA and the first purified terpene is Nerolidol

In one embodiment, the first purified cannabinoid is CBEVA and the first purified terpene is Beta-Myrcene.

In one embodiment, the first purified cannabinoid is CBEVA and the first purified terpene is Linalool.

In one embodiment, the first purified cannabinoid is CBEVA and the first purified terpene is Alpha-Caryophyllene.

In one embodiment, the first purified cannabinoid is CBEVA and the first purified terpene is Beta-Caryophyllene.

In one embodiment, the first purified cannabinoid is CBEVA and the first purified terpene is Alpha-Pinene.

In one embodiment, the first purified cannabinoid is CBEVA and the first purified terpene is Beta-Pinene.

In one embodiment, the first purified cannabinoid is CBEVA and the first purified terpene is Alpha-Bisabolol.

In one embodiment, the first purified cannabinoid is CBEVA and the first purified terpene is Delta-3-Carene.

In one embodiment, the first purified cannabinoid is CBEVA and the first purified terpene is Borneol.

In one embodiment, the first purified cannabinoid is CBEVA and the first purified terpene is Eucalyptol.

In one embodiment, the first purified cannabinoid is CBEVA and the first purified terpene is p-Cymene.

In one embodiment, the first purified cannabinoid is CBEVA and the first purified terpene is Alpha-Terpineol.

In one embodiment, the first purified cannabinoid is CBEVA and the first purified terpene is Alpha-Humulene.

In one embodiment, the first purified cannabinoid is CBEVA and the first purified terpene is Terpinolene.

In one embodiment, the first purified cannabinoid is CBEVA and the first purified terpene is Pulegone.

In one embodiment, the first purified cannabinoid is CBEVA and the first purified terpene is Camphene.

In one embodiment, the first purified cannabinoid is CBEVA and the first purified terpene is Geraniol.

In one embodiment of the compositions disclosed herein, the first purified cannabinoid and the first purified terpene comprise between 80-100% of the total mass of the composition.

In one embodiment of the compositions disclosed herein, the first purified cannabinoid and the first purified terpene comprise between 85-100% of the total mass of the composition.

In one embodiment of the compositions disclosed herein, the first purified cannabinoid and the first purified terpene comprise between 90-100% of the total mass of the composition.

In one embodiment of the compositions disclosed herein, the first purified cannabinoid and the first purified terpene comprise between 95-100% of the total mass of the composition.

In one embodiment of the compositions disclosed herein, the first purified cannabinoid and the first purified terpene comprise between 99-100% of the total mass of the composition.

In one embodiment of the disclosed composition, the ratio of the first purified cannabinoid to the first purified terpene is greater than 100:1, for example between 100:1 and 500:1.

In one embodiment of the disclosed composition, the ratio of the first purified cannabinoid to the first purified terpene is between 100:1 to 10:1.

In one embodiment of the disclosed composition, the ratio of purified cannabinoid to purified terpene is between 20:1 to 10:1.

In one embodiment of the disclosed composition, the ratio of purified cannabinoid to purified terpene is between 10:1 to 1:1.

In one embodiment of the disclosed composition, the ratio of purified cannabinoid to purified terpene is between 5:1 to 1:1.

In one embodiment of the disclosed composition, the ratio of purified cannabinoid to purified terpene is between 3:1 to 1:1.

In one embodiment of the disclosed composition, the ratio of purified cannabinoid to purified terpene is between 1:1 to 1:10.

In one embodiment of the disclosed composition, the ratio of purified cannabinoid to purified terpene is between 1:10 to 1:100.

In one embodiment, the compositions disclosed herein comprise a ratio of THC to purified Beta-Pinene of between 300:1 to 5:1.

In one embodiment, the compositions disclosed herein comprise a ratio of THC to purified Beta-Pinene of between 250:1 to 10:1.

In one embodiment, the compositions disclosed herein comprise a ratio of THC to purified Beta-Pinene of between 200:1 to 15:1.

In one embodiment, the compositions disclosed herein comprise a ratio of THC to purified Beta-Pinene of between 150:1 to 30:1.

In one embodiment, the compositions disclosed herein comprise a ratio of THC to purified Beta-Pinene of between 100:1 to 35:1.

In one embodiment, the compositions disclosed herein comprise a ratio of THC to purified Beta-Pinene of between 75:1 to 40:1.

In one embodiment, the compositions disclosed herein comprise a ratio of purified Beta-Pinene to CBG of greater than 2:1.

In one embodiment, the compositions disclosed herein comprise a ratio of purified Beta-Pinene to CBG of between 2:1 and 4:1.

In one embodiment, the compositions disclosed herein comprise a ratio of purified Beta-Pinene to CBG of between than 4:1 and 16:1.

In one embodiment, the compositions disclosed herein comprise a ratio of purified Beta-Pinene to CBG of between than 16:1 and 32:1.

In one embodiment, the compositions disclosed herein comprise a ratio of purified Beta-Pinene to CBG of between than 32:1 and 64:1.

In one embodiment, the compositions disclosed herein comprise a ratio of purified Beta-Pinene to CBG of greater than 100:1.

In one embodiment, the compositions disclosed herein comprise a ratio of THC to purified Eucalyptol of between 950:1 to 25:1.

In one embodiment, the compositions disclosed herein comprise a ratio of THC to purified Eucalyptol of between 875:1 to 35:1.

In one embodiment, the compositions disclosed herein comprise a ratio of THC to purified Eucalyptol of between 750:1 to 45:1.

In one embodiment, the compositions disclosed herein comprise a ratio of THC to purified Eucalyptol of between 625:1 to 55:1.

In one embodiment, the compositions disclosed herein comprise a ratio of THC to purified Eucalyptol of between 500:1 to 65:1.

In one embodiment, the compositions disclosed herein comprise a ratio of THC to purified Eucalyptol of between 350:1 to 75:1.

In one embodiment, the compositions disclosed herein comprise a ratio of THC to purified Eucalyptol of between 240:1 to 100:1.

In one embodiment, the compositions disclosed herein comprise a ratio of purified Eucalyptol to CBG of greater than 0.625:1.

In one embodiment, the compositions disclosed herein comprise a ratio of purified Eucalyptol to CBG of between 0.625:1 and 5:1.

In one embodiment, the compositions disclosed herein comprise a ratio of purified Eucalyptol to CBG of between 5:1 and 25:1.

In one embodiment, the compositions disclosed herein comprise a ratio of purified Eucalyptol to CBG of between 25:1 and 50:1.

In one embodiment, the compositions disclosed herein comprise a ratio of purified Eucalyptol to CBG of between 50:1 and 75:1.

In one embodiment, the compositions disclosed herein comprise a ratio of purified Eucalyptol to CBG of between 75:1 and 100:1.

In one embodiment, the compositions disclosed herein comprise a ratio of purified Eucalyptol to CBG of greater than 100:1.

In one embodiment, the compositions disclosed herein comprise a ratio of THC to purified Alpha-Humulene of between 400:1 to 10:1.

In one embodiment, the compositions disclosed herein comprise a ratio of THC to purified Alpha-Humulene of between 350:1 to 20:1.

In one embodiment, the compositions disclosed herein comprise a ratio of THC to purified Alpha-Humulene of between 300:1 to 25:1.

In one embodiment, the compositions disclosed herein comprise a ratio of THC to purified Alpha-Humulene of between 250:1 to 30:1.

In one embodiment, the compositions disclosed herein comprise a ratio of THC to purified Alpha-Humulene of between 200:1 to 35:1.

In one embodiment, the compositions disclosed herein comprise a ratio of THC to purified Alpha-Humulene of between 150:1 to 40:1.

In one embodiment, the compositions disclosed herein comprise a ratio of THC to purified Alpha-Humulene of between 100:1 to 50:1.

In one embodiment, the compositions disclosed herein comprise a ratio of purified Alpha-Humulene to CBG of greater than 1.5:1.

In one embodiment, the compositions disclosed herein comprise a ratio of purified Alpha-Humulene to CBG of between 1.5:1 and 3:1.

In one embodiment, the compositions disclosed herein comprise a ratio of purified Alpha-Humulene to CBG of between than 3:1 and 9:1.

In one embodiment, the compositions disclosed herein comprise a ratio of purified Alpha-Humulene to CBG of between than 9:1 and 27:1.

In one embodiment, the compositions disclosed herein comprise a ratio of purified Alpha-Humulene to CBG of between than 27:1 and 81:1.

In one embodiment, the compositions disclosed herein comprise a ratio of purified Alpha-Humulene to CBG of greater than 100:1.

In one embodiment, the compositions disclosed herein comprise a ratio of THC to purified Linalool of between 950:1 to 25:1.

In one embodiment, the compositions disclosed herein comprise a ratio of THC to purified Linalool of between 875:1 to 35:1.

In one embodiment, the compositions disclosed herein comprise a ratio of THC to purified Linalool of between 750:1 to 45:1.

In one embodiment, the compositions disclosed herein comprise a ratio of THC to purified Linalool of between 625:1 to 55:1.

In one embodiment, the compositions disclosed herein comprise a ratio of THC to purified Linalool of between 500:1 to 65:1.

In one embodiment, the compositions disclosed herein comprise a ratio of THC to purified Linalool of between 350:1 to 75:1.

In one embodiment, the compositions disclosed herein comprise a ratio of THC to purified Linalool of between 240:1 to 100:1.

In one embodiment, the compositions disclosed herein comprise a ratio of purified Linalool to CBG of greater than 0.625:1.

In one embodiment, the compositions disclosed herein comprise a ratio of purified Linalool to CBG of between 0.625:1 and 5:1.

In one embodiment, the compositions disclosed herein comprise a ratio of purified Linalool to CBG of between than 5:1 and 25:1.

In one embodiment, the compositions disclosed herein comprise a ratio of purified Linalool to CBG of between than 25:1 and 50:1.

In one embodiment, the compositions disclosed herein comprise a ratio of purified Linalool to CBG of between than 50:1 and 75:1.

In one embodiment, the compositions disclosed herein comprise a ratio of purified Linalool to CBG of between than 75:1 and 100:1.

In one embodiment, the compositions disclosed herein comprise a ratio of purified Linalool to CBG of greater than 100:1.

In one embodiment, the compositions disclosed herein comprise a ratio of THC to purified Limonene of between 350:1 to 10:1.

In one embodiment, the compositions disclosed herein comprise a ratio of THC to purified Limonene of between 300:1 to 15:1.

In one embodiment, the compositions disclosed herein comprise a ratio of THC to purified Limonene of between 250:1 to 20:1.

In one embodiment, the compositions disclosed herein comprise a ratio of THC to purified Limonene of between 200:1 to 25:1.

In one embodiment, the compositions disclosed herein comprise a ratio of THC to purified Limonene of between 150:1 to 30:1.

In one embodiment, the compositions disclosed herein comprise a ratio of THC to purified Limonene of between 100:1 to 35:1.

In one embodiment, the compositions disclosed herein comprise a ratio of THC to purified Limonene of between 85:1 to 40:1.

In one embodiment, the compositions disclosed herein comprise a ratio of purified Limonene to CBG of greater than 3.5:1.

In one embodiment, the compositions disclosed herein comprise a ratio of purified Limonene to CBG of between 3.5:1 and 7:1.

In one embodiment, the compositions disclosed herein comprise a ratio of purified Limonene to CBG of between than 7:1 and 25:1.

In one embodiment, the compositions disclosed herein comprise a ratio of purified Limonene to CBG of between than 25:1 and 50:1.

In one embodiment, the compositions disclosed herein comprise a ratio of purified Limonene to CBG of between than 50:1 and 75:1.

In one embodiment, the compositions disclosed herein comprise a ratio of purified Limonene to CBG of between than 75:1 and 100:1.

In one embodiment, the compositions disclosed herein comprise a ratio of purified Limonene to CBG of greater than 100:1.

In one embodiment, the compositions disclosed herein comprise a ratio of THC to purified Nerolidol of between 150:1 to 4:1.

In one embodiment, the compositions disclosed herein comprise a ratio of THC to purified Nerolidol of between 120:1 to 10:1.

In one embodiment, the compositions disclosed herein comprise a ratio of THC to purified Nerolidol of between 60:1 to 15:1.

In one embodiment, the compositions disclosed herein comprise a ratio of THC to purified Nerolidol of between 30:1 to 20:1.

In one embodiment, the compositions disclosed herein comprise a ratio of purified Nerolidol to CBG of greater than 3:1.

In one embodiment, the compositions disclosed herein comprise a ratio of purified Nerolidol to CBG of between 3:1 and 10:1.

In one embodiment, the compositions disclosed herein comprise a ratio of purified Nerolidol to CBG of between than 10:1 and 25:1.

In one embodiment, the compositions disclosed herein comprise a ratio of purified Nerolidol to CBG of between than 25:1 and 50:1.

In one embodiment, the compositions disclosed herein comprise a ratio of purified Nerolidol to CBG of between than 50:1 and 75:1.

In one embodiment, the compositions disclosed herein comprise a ratio of purified Nerolidol to CBG of between than 75:1 and 100:1.

In one embodiment, the compositions disclosed herein comprise a ratio of purified Nerolidol to CBG of greater than 100:1.

In one embodiment, the compositions disclosed herein comprise a ratio of THC to purified Beta-Caryophyllene of between 150:1 to 4:1.

In one embodiment, the compositions disclosed herein comprise a ratio of THC to purified Beta-Caryophyllene of between 120:1 to 5:1.

In one embodiment, the compositions disclosed herein comprise a ratio of THC to purified Beta-Caryophyllene of between 60:1 to 15:1.

In one embodiment, the compositions disclosed herein comprise a ratio of THC to purified Beta-Caryophyllene of between 30:1 to 20:1.

In one embodiment, the compositions disclosed herein comprise a ratio of purified Beta-Caryophyllene to CBG of greater than 3:1.

In one embodiment, the compositions disclosed herein comprise a ratio of purified Beta-Caryophyllene to CBG of between 3:1 and 10:1.

In one embodiment, the compositions disclosed herein comprise a ratio of purified Beta-Caryophyllene to CBG of between than 10:1 and 25:1.

In one embodiment, the compositions disclosed herein comprise a ratio of purified Beta-Caryophyllene to CBG of between than 25:1 and 50:1.

In one embodiment, the compositions disclosed herein comprise a ratio of purified Beta-Caryophyllene to CBG of between than 50:1 and 75:1.

In one embodiment, the compositions disclosed herein comprise a ratio of purified Beta-Caryophyllene to CBG of between than 75:1 and 100:1.

In one embodiment, the compositions disclosed herein comprise a ratio of purified Beta-Caryophyllene to CBG of greater than 100:1.

In one embodiment, the compositions disclosed herein comprise a ratio of THC to purified Alpha-Pinene of between 300:1 to 5:1.

In one embodiment, the compositions disclosed herein comprise a ratio of THC to purified Alpha-Pinene of between 250:1 to 10:1.

In one embodiment, the compositions disclosed herein comprise a ratio of THC to purified Alpha-Pinene of between 200:1 to 15:1.

In one embodiment, the compositions disclosed herein comprise a ratio of THC to purified Alpha-Pinene of between 150:1 to 30:1.

In one embodiment, the compositions disclosed herein comprise a ratio of THC to purified Alpha-Pinene of between 100:1 to 35:1.

In one embodiment, the compositions disclosed herein comprise a ratio of THC to purified Alpha-Pinene of between 75:1 to 40:1.

In one embodiment, the compositions disclosed herein comprise a ratio of purified Alpha-Pinene to CBG of greater than 2:1.

In one embodiment, the compositions disclosed herein comprise a ratio of purified Alpha-Pinene to CBG of between 2:1 and 4:1.

In one embodiment, the compositions disclosed herein comprise a ratio of purified Alpha-Pinene to CBG of between than 4:1 and 16:1.

In one embodiment, the compositions disclosed herein comprise a ratio of purified Alpha-Pinene to CBG of between than 16:1 and 32:1.

In one embodiment, the compositions disclosed herein comprise a ratio of purified Alpha-Pinene to CBG of between than 32:1 and 64:1.

In one embodiment, the compositions disclosed herein comprise a ratio of purified Alpha-Pinene to CBG of greater than 100:1.

In one embodiment, the compositions disclosed herein comprise a ratio of THC to purified Beta-Myrcene of between 200:1 to 3:1.

In one embodiment, the compositions disclosed herein comprise a ratio of THC to purified Beta-Myrcene of between 175:1 to 7.5:1.

In one embodiment, the compositions disclosed herein comprise a ratio of THC to purified Beta-Myrcene of between 150:1 to 10:1.

In one embodiment, the compositions disclosed herein comprise a ratio of THC to purified Beta-Myrcene of between 125:1 to 12.5:1.

In one embodiment, the compositions disclosed herein comprise a ratio of THC to purified Beta-Myrcene of between 100:1 to 15:1.

In one embodiment, the compositions disclosed herein comprise a ratio of THC to purified Beta-Myrcene of between 75:1 to 17.5:1.

In one embodiment, the compositions disclosed herein comprise a ratio of THC to purified Beta-Myrcene of between 50:1 to 20:1.

In one embodiment, the compositions disclosed herein comprise a ratio of purified Beta-Myrcene to CBG of greater than 3:1.

In one embodiment, the compositions disclosed herein comprise a ratio of purified Beta-Myrcene to CBG of between 3:1 and 10:1.

In one embodiment, the compositions disclosed herein comprise a ratio of purified Beta-Myrcene to CBG of between than 10:1 and 25:1.

In one embodiment, the compositions disclosed herein comprise a ratio of purified Beta-Myrcene to CBG of between than 25:1 and 50:1.

In one embodiment, the compositions disclosed herein comprise a ratio of purified Beta-Myrcene to CBG of between than 50:1 and 75:1.

In one embodiment, the compositions disclosed herein comprise a ratio of purified Beta-Myrcene to CBG of between than 75:1 and 100:1.

In one embodiment, the compositions disclosed herein comprise a ratio of purified Beta-Myrcene to CBG of greater than 100:1.

In one embodiment, the compositions disclosed herein comprise a ratio of THC to purified Alpha-Terpineol of between 2,000:1 to 50:1.

In one embodiment, the compositions disclosed herein comprise a ratio of THC to purified Alpha-Terpineol of between 1,750:1 to 75:1.

In one embodiment, the compositions disclosed herein comprise a ratio of THC to purified Alpha-Terpineol of between 1,500:1 to 100:1.

In one embodiment, the compositions disclosed herein comprise a ratio of THC to purified Alpha-Terpineol of between 1,250:1 to 125:1.

In one embodiment, the compositions disclosed herein comprise a ratio of THC to purified Alpha-Terpineol of between 1,000:1 to 150:1.

In one embodiment, the compositions disclosed herein comprise a ratio of THC to purified Alpha-Terpineol of between 750:1 to 175:1.

In one embodiment, the compositions disclosed herein comprise a ratio of THC to purified Alpha-Terpineol of between 500:1 to 200:1.

In one embodiment, the compositions disclosed herein comprise a ratio of purified Alpha-Terpineol to CBG of greater than 0.3:1.

In one embodiment, the compositions disclosed herein comprise a ratio of purified Alpha-Terpineol to CBG of between 0.3:1 and 1:1.

In one embodiment, the compositions disclosed herein comprise a ratio of purified Alpha-Terpineol to CBG of between 0.3:1 and 5:1.

In one embodiment, the compositions disclosed herein comprise a ratio of purified Alpha-Terpineol to CBG of between than 5:1 and 25:1.

In one embodiment, the compositions disclosed herein comprise a ratio of purified Alpha-Terpineol to CBG of between than 25:1 and 50:1.

In one embodiment, the compositions disclosed herein comprise a ratio of purified Alpha-Terpineol to CBG of between than 50:1 and 75:1.

In one embodiment, the compositions disclosed herein comprise a ratio of purified Alpha-Terpineol to CBG of between than 75:1 and 100:1.

In one embodiment, the compositions disclosed herein comprise a ratio of purified Alpha-Terpineol to CBG of greater than 100:1.

In one embodiment, the compositions disclosed herein comprise a ratio of THC to purified Terpinolene of between 1,200:1 to 30:1.

In one embodiment, the compositions disclosed herein comprise a ratio of THC to purified Terpinolene of between 1,000:1 to 45:1.

In one embodiment, the compositions disclosed herein comprise a ratio of THC to purified Terpinolene of between 900:1 to 60:1.

In one embodiment, the compositions disclosed herein comprise a ratio of THC to purified Terpinolene of between 750:1 to 75:1.

In one embodiment, the compositions disclosed herein comprise a ratio of THC to purified Terpinolene of between 600:1 to 90:1.

In one embodiment, the compositions disclosed herein comprise a ratio of THC to purified Terpinolene of between 425:1 to 105:1.

In one embodiment, the compositions disclosed herein comprise a ratio of THC to purified Terpinolene of between 350:1 to 120:1.

In one embodiment, the compositions disclosed herein comprise a ratio of purified Terpinolene to CBG of greater than 0.5:1.

In one embodiment, the compositions disclosed herein comprise a ratio of purified Terpinolene to CBG of between 0.5:1 and 1:1.

In one embodiment, the compositions disclosed herein comprise a ratio of purified Terpinolene to CBG of between 1:1 and 5:1.

In one embodiment, the compositions disclosed herein comprise a ratio of purified Terpinolene to CBG of between 5:1 and 25:1.

In one embodiment, the compositions disclosed herein comprise a ratio of purified Terpinolene to CBG of between 25:1 and 50:1.

In one embodiment, the compositions disclosed herein comprise a ratio of purified Terpinolene to CBG of between 50:1 and 75:1.

In one embodiment, the compositions disclosed herein comprise a ratio of purified Terpinolene to CBG of between 75:1 and 100:1.

In one embodiment, the compositions disclosed herein comprise a ratio of purified Terpinolene to CBG of greater than 100:1.

In one embodiment, the compositions disclosed herein comprise a ratio of THC to purified Pulegone of between 1,200:1 to 30:1.

In one embodiment, the compositions disclosed herein comprise a ratio of THC to purified Pulegone of between 1,000:1 to 45:1.

In one embodiment, the compositions disclosed herein comprise a ratio of THC to purified Pulegone of between 900:1 to 60:1.

In one embodiment, the compositions disclosed herein comprise a ratio of THC to purified Pulegone of between 750:1 to 75:1.

In one embodiment, the compositions disclosed herein comprise a ratio of THC to purified Pulegone of between 600:1 to 90:1.

In one embodiment, the compositions disclosed herein comprise a ratio of THC to purified Pulegone of between 425:1 to 105:1.

In one embodiment, the compositions disclosed herein comprise a ratio of THC to purified Pulegone of between 350:1 to 120:1.

In one embodiment, the compositions disclosed herein comprise a ratio of purified Pulegone to CBG of greater than 0.5:1.

In one embodiment, the compositions disclosed herein comprise a ratio of purified Pulegone to CBG of between 0.5:1 and 1:1.

In one embodiment, the compositions disclosed herein comprise a ratio of purified Pulegone to CBG of between 1:1 and 5:1.

In one embodiment, the compositions disclosed herein comprise a ratio of purified Pulegone to CBG of between 5:1 and 25:1.

In one embodiment, the compositions disclosed herein comprise a ratio of purified Pulegone to CBG of between 25:1 and 50:1.

In one embodiment, the compositions disclosed herein comprise a ratio of purified Pulegone to CBG of between 50:1 and 75:1.

In one embodiment, the compositions disclosed herein comprise a ratio of purified Pulegone to CBG of between 75:1 and 100:1.

In one embodiment, the compositions disclosed herein comprise a ratio of purified Pulegone to CBG of greater than 100:1.

In one embodiment, the compositions disclosed herein comprise a ratio of THC to purified Camphene of between 950:1 to 25:1.

In one embodiment, the compositions disclosed herein comprise a ratio of THC to purified Camphene of between 875:1 to 35:1.

In one embodiment, the compositions disclosed herein comprise a ratio of THC to purified Camphene of between 750:1 to 45:1.

In one embodiment, the compositions disclosed herein comprise a ratio of THC to purified Camphene of between 625:1 to 55:1.

In one embodiment, the compositions disclosed herein comprise a ratio of THC to purified Camphene of between 500:1 to 65:1.

In one embodiment, the compositions disclosed herein comprise a ratio of THC to purified Camphene of between 350:1 to 75:1.

In one embodiment, the compositions disclosed herein comprise a ratio of THC to purified Camphene of between 240:1 to 100:1.

In one embodiment, the compositions disclosed herein comprise a ratio of purified Camphene to CBG of greater than 0.625:1.

In one embodiment, the compositions disclosed herein comprise a ratio of purified Camphene to CBG of between 0.625:1 and 5:1.

In one embodiment, the compositions disclosed herein comprise a ratio of purified Camphene to CBG of between than 5:1 and 25:1.

In one embodiment, the compositions disclosed herein comprise a ratio of purified Camphene to CBG of between than 25:1 and 50:1.

In one embodiment, the compositions disclosed herein comprise a ratio of purified Camphene to CBG of between than 50:1 and 75:1.

In one embodiment, the compositions disclosed herein comprise a ratio of purified Camphene to CBG of between than 75:1 and 100:1.

In one embodiment, the compositions disclosed herein comprise a ratio of purified Camphene to CBG of greater than 100:1.

In one embodiment, the compositions disclosed herein comprise a ratio of THC to purified Delta-3-Carene of between 950:1 to 25:1.

In one embodiment, the compositions disclosed herein comprise a ratio of THC to purified Delta-3-Carene of between 875:1 to 35:1.

In one embodiment, the compositions disclosed herein comprise a ratio of THC to purified Delta-3-Carene of between 750:1 to 45:1.

In one embodiment, the compositions disclosed herein comprise a ratio of THC to purified Delta-3-Carene of between 625:1 to 55:1.

In one embodiment, the compositions disclosed herein comprise a ratio of THC to purified Delta-3-Carene of between 500:1 to 65:1.

In one embodiment, the compositions disclosed herein comprise a ratio of THC to purified Delta-3-Carene of between 350:1 to 75:1.

In one embodiment, the compositions disclosed herein comprise a ratio of THC to purified Delta-3-Carene of between 240:1 to 100:1.

In one embodiment, the compositions disclosed herein comprise a ratio of purified Delta-3-Carene to CBG of greater than 0.625:1.

In one embodiment, the compositions disclosed herein comprise a ratio of purified Delta-3-Carene to CBG of between 0.625:1 and 5:1.

In one embodiment, the compositions disclosed herein comprise a ratio of purified Delta-3-Carene to CBG of between than 5:1 and 25:1.

In one embodiment, the compositions disclosed herein comprise a ratio of purified Delta-3-Carene to CBG of between than 25:1 and 50:1.

In one embodiment, the compositions disclosed herein comprise a ratio of purified Delta-3-Carene to CBG of between than 50:1 and 75:1

In one embodiment, the compositions disclosed herein comprise a ratio of purified Delta-3-Carene to CBG of between than 75:1 and 100:1.

In one embodiment, the compositions disclosed herein comprise a ratio of purified Delta-3-Carene to CBG of greater than 100:1.

In one embodiment, the compositions disclosed herein comprise a ratio of THC to purified Geraniol of between 950:1 to 25:1.

In one embodiment, the compositions disclosed herein comprise a ratio of THC to purified Geraniol of between 875:1 to 35:1.

In one embodiment, the compositions disclosed herein comprise a ratio of THC to purified Geraniol of between 750:1 to 45:1.

In one embodiment, the compositions disclosed herein comprise a ratio of THC to purified Geraniol of between 625:1 to 55:1.

In one embodiment, the compositions disclosed herein comprise a ratio of THC to purified Geraniol of between 500:1 to 65:1.

In one embodiment, the compositions disclosed herein comprise a ratio of THC to purified Geraniol of between 350:1 to 75:1.

In one embodiment, the compositions disclosed herein comprise a ratio of THC to purified Geraniol of between 240:1 to 100:1.

In one embodiment, the compositions disclosed herein comprise a ratio of purified Geraniol to CBG of greater than 0.625:1.

In one embodiment, the compositions disclosed herein comprise a ratio of purified Geraniol to CBG of between 0.625:1 and 5:1.

In one embodiment, the compositions disclosed herein comprise a ratio of purified Geraniol to CBG of between than 5:1 and 25:1.

In one embodiment, the compositions disclosed herein comprise a ratio of purified Geraniol to CBG of between than 25:1 and 50:1.

In one embodiment, the compositions disclosed herein comprise a ratio of purified Geraniol to CBG of between than 50:1 and 75:1.

In one embodiment, the compositions disclosed herein comprise a ratio of purified Geraniol to CBG of between than 75:1 and 100:1.

In one embodiment, the compositions disclosed herein comprise a ratio of purified Geraniol to CBG of greater than 100:1.

In one embodiment, the compositions disclosed herein comprise a ratio of THC to purified p-Cymene of between 950:1 to 25:1.

In one embodiment, the compositions disclosed herein comprise a ratio of THC to purified p-Cymene of between 875:1 to 35:1.

In one embodiment, the compositions disclosed herein comprise a ratio of THC to purified p-Cymene of between 750:1 to 45:1.

In one embodiment, the compositions disclosed herein comprise a ratio of THC to purified p-Cymene of between 625:1 to 55:1.

In one embodiment, the compositions disclosed herein comprise a ratio of THC to purified p-Cymene of between 500:1 to 65:1.

In one embodiment, the compositions disclosed herein comprise a ratio of THC to purified p-Cymene of between 350:1 to 75:1.

In one embodiment, the compositions disclosed herein comprise a ratio of THC to purified p-Cymene of between 240:1 to 100:1.

In one embodiment, the compositions disclosed herein comprise a ratio of purified p-Cymene to CBG of greater than 0.625:1.

In one embodiment, the compositions disclosed herein comprise a ratio of purified p-Cymene to CBG of between 0.625:1 and 5:1.

In one embodiment, the compositions disclosed herein comprise a ratio of purified p-Cymene to CBG of between than 5:1 and 25:1.

In one embodiment, the compositions disclosed herein comprise a ratio of purified p-Cymene to CBG of between than 25:1 and 50:1.

In one embodiment, the compositions disclosed herein comprise a ratio of purified p-Cymene to CBG of between than 50:1 and 75:1.

In one embodiment, the compositions disclosed herein comprise a ratio of purified p-Cymene to CBG of between than 75:1 and 100:1.

In one embodiment, the compositions disclosed herein comprise a ratio of purified p-Cymene to CBG of greater than 100:1.

In one embodiment, the above described combinations of a first purified cannabinoid and a first purified terpene include a second purified cannabinoid chosen from THC, D9-THC, D8-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

Accordingly, disclosed herein are new compositions comprising unique combinations of a first purified cannabinoid, a second purified cannabinoid, and a first purified terpene. In one embodiment, the compositions disclosed herein comprise a first purified cannabinoid, a second purified cannabinoid, and a first purified terpene present in non-naturally occurring concentrations. In one embodiment, the compositions disclosed herein comprise a first purified cannabinoid, a second purified cannabinoid, and a first purified terpene present in non-naturally occurring ratios. In one embodiment, the compositions disclosed herein provide particular benefits previously unavailable with naturally occurring cannabinoid profiles, such as those found in plants, harvested flowers, extracts, or conventional products derived from the same.

In one embodiment of the disclosed composition, the ratio of the first purified cannabinoid to the second purified cannabinoid to the first purified terpene is greater than 100:1:1.

In one embodiment of the compositions disclosed herein, the ratio of the first purified cannabinoid to the second purified cannabinoid to the first purified terpene is between 100:1:1 to 10:1:1.

In one embodiment of the compositions disclosed herein, the ratio of the first purified cannabinoid to the second purified cannabinoid to the first purified terpene is between 20:1:1 to 10:1:1.

In one embodiment of the compositions disclosed herein, the ratio of the first purified cannabinoid to the second purified cannabinoid to the first purified terpene is between 10:1:1 to 1:1:1.

In one embodiment of the compositions disclosed herein, the ratio of the first purified cannabinoid to the second purified cannabinoid to the first purified terpene is between 5:1:1 to 1:1:1.

In one embodiment of the compositions disclosed herein, the ratio of the first purified cannabinoid to the second purified cannabinoid to the first purified terpene is between 3:1:1 to 1:1:1.

In one embodiment of the compositions disclosed herein, the ratio of the first purified cannabinoid to the second purified cannabinoid to the first purified terpene is between 1:1:1 to 1:1:10.

In one embodiment of the compositions disclosed herein, the ratio of the first purified cannabinoid to the second purified cannabinoid to the first purified terpene is between 1:1:10 to 1:1:100.

In one embodiment of the compositions disclosed herein, the ratio of the first purified cannabinoid to the second purified cannabinoid to the first purified terpene is between 100:50:1 to 10:5:1.

In one embodiment of the compositions disclosed herein, the ratio of the first purified cannabinoid to the second purified cannabinoid to the first purified terpene is between 50:10:1 to 5:2:1.

In one embodiment of the compositions disclosed herein, the ratio of the first purified cannabinoid to the second purified cannabinoid to the first purified terpene is between 25:15:1 to 1:1:1.

In one embodiment of the compositions disclosed herein, the ratio of the first purified cannabinoid to the second purified cannabinoid to the first purified terpene is between 1:1:1 to 1:5:5.

In one embodiment of the compositions disclosed herein, the ratio of the first purified cannabinoid to the second purified cannabinoid to the first purified terpene is between 1:5:5 to 1:25:100.

In one embodiment of the compositions disclosed herein, the ratio of the first purified cannabinoid to the second purified cannabinoid to the first purified terpene is between 100:100:1 to 10:10:1.

In one embodiment of the compositions disclosed herein, the ratio of the first purified cannabinoid to the second purified cannabinoid to the first purified terpene is between 20:20:1 to 10:10:1.

In one embodiment of the compositions disclosed herein, the ratio of the first purified cannabinoid to the second purified cannabinoid to the first purified terpene is between 10:10:1 to 1:1:1.

In one embodiment of the compositions disclosed herein, the ratio of the first purified cannabinoid to the second purified cannabinoid to the first purified terpene is between 5:5:1 to 1:1:1.

In one embodiment of the compositions disclosed herein, the ratio of the first purified cannabinoid to the second purified cannabinoid to the first purified terpene is between 3:3:1 to 1:1:1.

In one embodiment of the compositions disclosed herein, the ratio of the first purified cannabinoid to the second purified cannabinoid to the first purified terpene is between 1:1:1 to 1:10:10.

In one embodiment of the compositions disclosed herein, the ratio of the first purified cannabinoid to the second purified cannabinoid to the first purified terpene is between 1:10:10 to 1:100:100.

In one embodiment of the disclosed composition, the first purified cannabinoid, the second purified cannabinoid, and the first purified terpene, together, comprise between 80-100% of the total mass of the composition.

In one embodiment of the disclosed composition, the first purified cannabinoid, the second purified cannabinoid, and the first purified terpene, together, comprise between 85-100% of the total mass of the composition.

In one embodiment of the disclosed composition, the first purified cannabinoid, the second purified cannabinoid, and the first purified terpene, together, comprise between 90-100% of the total mass of the composition.

In one embodiment of the disclosed composition, the first purified cannabinoid, the second purified cannabinoid, and the first purified terpene, together, comprise between 95-100% of the total mass of the composition.

In one embodiment of the disclosed composition, the first purified cannabinoid, the second purified cannabinoid, and the first purified terpene, together, comprise between 99-100% of the total mass of the composition.

In one embodiment, the above described combinations of a first purified cannabinoid and a first purified terpene include a second purified terpene chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol.

Accordingly, disclosed herein are new compositions comprising unique combinations of a first purified cannabinoid, a first purified terpene, and a second purified terpene are present in non-naturally occurring concentrations. In one embodiment, the first purified cannabinoid, the first purified terpene, and second purified terpene are present in non-naturally occurring ratios. In one embodiment, the compositions disclosed herein provide particular benefits previously unavailable with naturally occurring cannabinoid profiles, such as those found in plants, harvested flowers, extracts, or conventional products derived from the same.

In one embodiment of the disclosed composition, the ratio of the first purified cannabinoid to the first purified terpene to the second purified terpene is greater than 100:1:1.

In one embodiment of the compositions disclosed herein, the ratio of the first purified cannabinoid to the first purified terpene to the second purified terpene is between 100:1:1 to 10:1:1.

In one embodiment of the compositions disclosed herein, the ratio of the first purified cannabinoid to the first purified terpene to the second purified terpene is between 20:1:1 to 10:1:1.

In one embodiment of the compositions disclosed herein, the ratio of the first purified cannabinoid to the first purified terpene to the second purified terpene is between 10:1:1 to 1:1:1.

In one embodiment of the compositions disclosed herein, the ratio of the first purified cannabinoid to the first purified terpene to the second purified terpene is between 5:1:1 to 1:1:1.

In one embodiment of the compositions disclosed herein, the ratio of the first purified cannabinoid to the first purified terpene to the second purified terpene is between 3:1:1 to 1:1:1.

In one embodiment of the compositions disclosed herein, the ratio of the first purified cannabinoid to the first purified terpene to the second purified terpene is between 1:1:1 to 1:1:10.

In one embodiment of the compositions disclosed herein, the ratio of the first purified cannabinoid to the first purified terpene to the second purified terpene is between 1:1:10 to 1:1:100.

In one embodiment of the compositions disclosed herein, the ratio of the first purified cannabinoid to the first purified terpene to the second purified terpene is between 100:50:1 to 10:5:1.

In one embodiment of the compositions disclosed herein, the ratio of the first purified cannabinoid to the first purified terpene to the second purified terpene is between 50:10:1 to 5:2:1.

In one embodiment of the compositions disclosed herein, the ratio of the first purified cannabinoid to the first purified terpene to the second purified terpene is between 25:15:1 to 1:1:1.

In one embodiment of the compositions disclosed herein, the ratio of the first purified cannabinoid to the first purified terpene to the second purified terpene is between 1:1:1 to 1:5:5.

In one embodiment of the compositions disclosed herein, the ratio of the first purified cannabinoid to the first purified terpene to the second purified terpene is between 1:5:5 to 1:25:100.

In one embodiment of the compositions disclosed herein, the ratio of the first purified cannabinoid to the first purified terpene to the second purified terpene is between 100:100:1 to 10:10:1.

In one embodiment of the compositions disclosed herein, the ratio of the first purified cannabinoid to the first purified terpene to the second purified terpene is between 20:20:1 to 10:10:1.

In one embodiment of the compositions disclosed herein, the ratio of the first purified cannabinoid to the first purified terpene to the second purified terpene is between 10:10:1 to 1:1:1.

In one embodiment of the compositions disclosed herein, the ratio of the first purified cannabinoid to the first purified terpene to the second purified terpene is between 5:5:1 to 1:1:1.

In one embodiment of the compositions disclosed herein, the ratio of the first purified cannabinoid to the first purified terpene to the second purified terpene is between 3:3:1 to 1:1:1.

In one embodiment of the compositions disclosed herein, the ratio of the first purified cannabinoid to the first purified terpene to the second purified terpene is between 1:1:1 to 1:10:10.

In one embodiment of the compositions disclosed herein, the ratio of the first purified cannabinoid to the first purified terpene to the second purified terpene is between 1:10:10 to 1:100:100.

In one embodiment of the disclosed composition, the ratio of the first purified cannabinoid to the first purified terpene to the second purified terpene, together, comprise between 80-100% of the total mass of the composition.

In one embodiment of the disclosed composition, the ratio of the first purified cannabinoid to the first purified terpene to the second purified terpene, together, comprise between 85-100% of the total mass of the composition.

In one embodiment of the disclosed composition, the ratio of the first purified cannabinoid to the first purified terpene to the second purified terpene, together, comprise between 90-100% of the total mass of the composition.

In one embodiment of the disclosed composition, the ratio of the first purified cannabinoid to the first purified terpene to the second purified terpene, together, comprise between 95-100% of the total mass of the composition.

In one embodiment of the disclosed composition, the ratio of the first purified cannabinoid to the first purified terpene to the second purified terpene, together, comprise between 99-100% of the total mass of the composition.

Disclosed herein are new compositions comprising combinations of a first purified cannabinoid and a second purified cannabinoid. In one embodiment, compositions disclosed herein are manmade. In one embodiment, the first purified cannabinoid and the second purified cannabinoid are present in non-naturally occurring concentrations. In one embodiment, the first purified cannabinoid and the second purified cannabinoid are present in non-naturally occurring ratios. In one embodiment, the compositions disclosed herein provide particular benefits previously unavailable with naturally occurring cannabinoid profiles, such as those found in plants, harvested flowers, extracts, or conventional products derived from the same.

In one embodiment, the said combination of a first purified cannabinoid and a second purified cannabinoid provide a synergistic effect at a mammalian cellular receptor, such as a G protein coupled receptor. In one embodiment, the receptor is CB1. In one embodiment, the receptor is CB2. In one embodiment, the receptor is GPR55. In one embodiment, the receptor is 5HT-1A. In one embodiment, the receptor is 5HT-2A. In one embodiment, the receptor is TRPV1. In one embodiment, the receptor is an opioid receptor, e.g., μ-opioid receptor.

In one embodiment of the compositions disclosed herein, the ratio of the first purified cannabinoid to the second purified cannabinoid is greater than 100:1.

In one embodiment of the compositions disclosed herein, the ratio of the first purified cannabinoid to the second purified cannabinoid is between 100:1 to 10:1.

In one embodiment of the compositions disclosed herein, the ratio of the first purified cannabinoid to the second purified cannabinoid is between 20:1 to 10:1.

In one embodiment of the compositions disclosed herein, the ratio of the first purified cannabinoid to the second purified cannabinoid is between 10:1 to 1:1.

In one embodiment of the compositions disclosed herein, the ratio of the first purified cannabinoid to the second purified cannabinoid is between 5:1 to 1:1.

In one embodiment of the compositions disclosed herein, the ratio of the first purified cannabinoid to the second purified cannabinoid is between 3:1 to 1:1.

In one embodiment of the compositions disclosed herein, the ratio of the first purified cannabinoid to the second purified cannabinoid is between 1:1 to 1:10.

In one embodiment of the compositions disclosed herein, the first purified cannabinoid and the second purified cannabinoid comprise between 80-100% of the total mass of the composition.

In one embodiment of the compositions disclosed herein, the first purified cannabinoid and the second purified cannabinoid comprise between 85-100% of the total mass of the composition.

In one embodiment of the compositions disclosed herein, the first purified cannabinoid and the second purified cannabinoid comprise between 90-100% of the total mass of the composition.

In one embodiment of the compositions disclosed herein, the first purified cannabinoid and the second purified cannabinoid comprise between 95-100% of the total mass of the composition.

In one embodiment of the compositions disclosed herein, the first purified cannabinoid and the second purified cannabinoid comprise between 99-100% of the total mass of the composition.

In one embodiment of the compositions disclosed herein, the first purified cannabinoid is D9-THC and the second purified cannabinoid is D8-THC.

In one embodiment of the compositions disclosed herein, the first purified cannabinoid is D9-THC and the second purified cannabinoid is THCA.

In one embodiment of the compositions disclosed herein, the first purified cannabinoid is D9-THC and the second purified cannabinoid is THCV.

In one embodiment of the compositions disclosed herein, the first purified cannabinoid is D9-THC and the second purified cannabinoid is THCVA.

In one embodiment of the compositions disclosed herein, the first purified cannabinoid is D9-THC and the second purified cannabinoid is CBD.

In one embodiment of the compositions disclosed herein, the first purified cannabinoid is D9-THC and the second purified cannabinoid is CBDA.

In one embodiment of the compositions disclosed herein, the first purified cannabinoid is D9-THC and the second purified cannabinoid is CBDV.

In one embodiment of the compositions disclosed herein, the first purified cannabinoid is D9-THC and the second purified cannabinoid is CBDVA.

In one embodiment of the compositions disclosed herein, the first purified cannabinoid is D9-THC and the second purified cannabinoid is CBC.

In one embodiment of the compositions disclosed herein, the first purified cannabinoid is D9-THC and the second purified cannabinoid is CBCA.

In one embodiment of the compositions disclosed herein, the first purified cannabinoid is D9-THC and the second purified cannabinoid is CBCV.

In one embodiment of the compositions disclosed herein, the first purified cannabinoid is D9-THC and the second purified cannabinoid is CBCVA.

In one embodiment of the compositions disclosed herein, the first purified cannabinoid is D9-THC and the second purified cannabinoid is CBG.

In one embodiment of the compositions disclosed herein, the first purified cannabinoid is D9-THC and the second purified cannabinoid is CBGA.

In one embodiment of the compositions disclosed herein, the first purified cannabinoid is D9-THC and the second purified cannabinoid is CBGV.

In one embodiment of the compositions disclosed herein, the first purified cannabinoid is D9-THC and the second purified cannabinoid is CBGVA.

In one embodiment of the compositions disclosed herein, the first purified cannabinoid is D9-THC and the second purified cannabinoid is CBN.

In one embodiment of the compositions disclosed herein, the first purified cannabinoid is D9-THC and the second purified cannabinoid is CBNA.

In one embodiment of the compositions disclosed herein, the first purified cannabinoid is D9-THC and the second purified cannabinoid is CBNV.

In one embodiment of the compositions disclosed herein, the first purified cannabinoid is D9-THC and the second purified cannabinoid is CBNVA.

In one embodiment of the compositions disclosed herein, the first purified cannabinoid is D8-THC and the second purified cannabinoid is THCA.

In one embodiment of the compositions disclosed herein, the first purified cannabinoid is D8-THC and the second purified cannabinoid is THCV.

In one embodiment of the compositions disclosed herein, the first purified cannabinoid is D8-THC and the second purified cannabinoid is THCVA.

In one embodiment of the compositions disclosed herein, the first purified cannabinoid is D8-THC and the second purified cannabinoid is CBD.

In one embodiment of the compositions disclosed herein, the first purified cannabinoid is D8-THC and the second purified cannabinoid is CBDA.

In one embodiment of the compositions disclosed herein, the first purified cannabinoid is D8-THC and the second purified cannabinoid is CBDV.

In one embodiment of the compositions disclosed herein, the first purified cannabinoid is D8-THC and the second purified cannabinoid is CBDVA.

In one embodiment of the compositions disclosed herein, the first purified cannabinoid is D8-THC and the second purified cannabinoid is CBC.

In one embodiment of the compositions disclosed herein, the first purified cannabinoid is D8-THC and the second purified cannabinoid is CBCA.

In one embodiment of the compositions disclosed herein, the first purified cannabinoid is D8-THC and the second purified cannabinoid is CBCV.

In one embodiment of the compositions disclosed herein, the first purified cannabinoid is D8-THC and the second purified cannabinoid is CBCVA.

In one embodiment of the compositions disclosed herein, the first purified cannabinoid is D8-THC and the second purified cannabinoid is CBG.

In one embodiment of the compositions disclosed herein, the first purified cannabinoid is D8-THC and the second purified cannabinoid is CBGA.

In one embodiment of the compositions disclosed herein, the first purified cannabinoid is D8-THC and the second purified cannabinoid is CBGV.

In one embodiment of the compositions disclosed herein, the first purified cannabinoid is D8-THC and the second purified cannabinoid is CBGVA.

In one embodiment of the compositions disclosed herein, the first purified cannabinoid is D8-THC and the second purified cannabinoid is CBN.

In one embodiment of the compositions disclosed herein, the first purified cannabinoid is D8-THC and the second purified cannabinoid is CBNA.

In one embodiment of the compositions disclosed herein, the first purified cannabinoid is D8-THC and the second purified cannabinoid is CBNV.

In one embodiment of the compositions disclosed herein, the first purified cannabinoid is D8-THC and the second purified cannabinoid is CBNVA.

In one embodiment of the compositions disclosed herein, the first purified cannabinoid is THCA and the second purified cannabinoid is THCV.

In one embodiment of the compositions disclosed herein, the first purified cannabinoid is THCA and the second purified cannabinoid is THCVA.

In one embodiment of the compositions disclosed herein, the first purified cannabinoid is THCA and the second purified cannabinoid is CBD.

In one embodiment of the compositions disclosed herein, the first purified cannabinoid is THCA and the second purified cannabinoid is CBDA.

In one embodiment of the compositions disclosed herein, the first purified cannabinoid is THCA and the second purified cannabinoid is CBDV.

In one embodiment of the compositions disclosed herein, the first purified cannabinoid is THCA and the second purified cannabinoid is CBDVA.

In one embodiment of the compositions disclosed herein, the first purified cannabinoid is THCA and the second purified cannabinoid is CBC.

In one embodiment of the compositions disclosed herein, the first purified cannabinoid is THCA and the second purified cannabinoid is CBCA.

In one embodiment of the compositions disclosed herein, the first purified cannabinoid is THCA and the second purified cannabinoid is CBCV.

In one embodiment of the compositions disclosed herein, the first purified cannabinoid is THCA and the second purified cannabinoid is CBCVA.

In one embodiment of the compositions disclosed herein, the first purified cannabinoid is THCA and the second purified cannabinoid is CBG.

In one embodiment of the compositions disclosed herein, the first purified cannabinoid is THCA and the second purified cannabinoid is CBGA.

In one embodiment of the compositions disclosed herein, the first purified cannabinoid is THCA and the second purified cannabinoid is CBGV.

In one embodiment of the compositions disclosed herein, the first purified cannabinoid is THCA and the second purified cannabinoid is CBGVA.

In one embodiment of the compositions disclosed herein, the first purified cannabinoid is THCA and the second purified cannabinoid is CBN.

In one embodiment of the compositions disclosed herein, the first purified cannabinoid is THCA and the second purified cannabinoid is CBNA.

In one embodiment of the compositions disclosed herein, the first purified cannabinoid is THCA and the second purified cannabinoid is CBNV.

In one embodiment of the compositions disclosed herein, the first purified cannabinoid is THCA and the second purified cannabinoid is CBNVA.

In one embodiment of the compositions disclosed herein, the first purified cannabinoid is THCV and the second purified cannabinoid is THCVA.

In one embodiment of the compositions disclosed herein, the first purified cannabinoid is THCV and the second purified cannabinoid is CBD.

In one embodiment of the compositions disclosed herein, the first purified cannabinoid is THCV and the second purified cannabinoid is CBDA.

In one embodiment of the compositions disclosed herein, the first purified cannabinoid is THCV and the second purified cannabinoid is CBDV.

In one embodiment of the compositions disclosed herein, the first purified cannabinoid is THCV and the second purified cannabinoid is CBDVA.

In one embodiment of the compositions disclosed herein, the first purified cannabinoid is THCV and the second purified cannabinoid is CBC.

In one embodiment of the compositions disclosed herein, the first purified cannabinoid is THCV and the second purified cannabinoid is CBCA.

In one embodiment of the compositions disclosed herein, the first purified cannabinoid is THCV and the second purified cannabinoid is CBCV.

In one embodiment of the compositions disclosed herein, the first purified cannabinoid is THCV and the second purified cannabinoid is CBCVA.

In one embodiment of the compositions disclosed herein, the first purified cannabinoid is THCV and the second purified cannabinoid is CBG.

In one embodiment of the compositions disclosed herein, the first purified cannabinoid is THCV and the second purified cannabinoid is CBGA.

In one embodiment of the compositions disclosed herein, the first purified cannabinoid is THCV and the second purified cannabinoid is CBGV.

In one embodiment of the compositions disclosed herein, the first purified cannabinoid is THCV and the second purified cannabinoid is CBGVA.

In one embodiment of the compositions disclosed herein, the first purified cannabinoid is THCV and the second purified cannabinoid is CBN.

In one embodiment of the compositions disclosed herein, the first purified cannabinoid is THCV and the second purified cannabinoid is CBNA.

In one embodiment of the compositions disclosed herein, the first purified cannabinoid is THCV and the second purified cannabinoid is CBNV.

In one embodiment of the compositions disclosed herein, the first purified cannabinoid is THCV and the second purified cannabinoid is CBNVA.

In one embodiment of the compositions disclosed herein, the first purified cannabinoid is THCVA and the second purified cannabinoid is CBD.

In one embodiment of the compositions disclosed herein, the first purified cannabinoid is THCVA and the second purified cannabinoid is CBDA.

In one embodiment of the compositions disclosed herein, the first purified cannabinoid is THCVA and the second purified cannabinoid is CBDV.

In one embodiment of the compositions disclosed herein, the first purified cannabinoid is THCVA and the second purified cannabinoid is CBDVA.

In one embodiment of the compositions disclosed herein, the first purified cannabinoid is THCVA and the second purified cannabinoid is CBC.

In one embodiment of the compositions disclosed herein, the first purified cannabinoid is THCVA and the second purified cannabinoid is CBCA.

In one embodiment of the compositions disclosed herein, the first purified cannabinoid is THCVA and the second purified cannabinoid is CBCV.

In one embodiment of the compositions disclosed herein, the first purified cannabinoid is THCVA and the second purified cannabinoid is CBCVA.

In one embodiment of the compositions disclosed herein, the first purified cannabinoid is THCVA and the second purified cannabinoid is CBG.

In one embodiment of the compositions disclosed herein, the first purified cannabinoid is THCVA and the second purified cannabinoid is CBGA.

In one embodiment of the compositions disclosed herein, the first purified cannabinoid is THCVA and the second purified cannabinoid is CBGV.

In one embodiment of the compositions disclosed herein, the first purified cannabinoid is THCVA and the second purified cannabinoid is CBGVA.

In one embodiment of the compositions disclosed herein, the first purified cannabinoid is THCVA and the second purified cannabinoid is CBN.

In one embodiment of the compositions disclosed herein, the first purified cannabinoid is THCVA and the second purified cannabinoid is CBNA.

In one embodiment of the compositions disclosed herein, the first purified cannabinoid is THCVA and the second purified cannabinoid is CBNV.

In one embodiment of the compositions disclosed herein, the first purified cannabinoid is THCVA and the second purified cannabinoid is CBNVA.

In one embodiment of the compositions disclosed herein, the first purified cannabinoid is CBD and the second purified cannabinoid is CBDA.

In one embodiment of the compositions disclosed herein, the first purified cannabinoid is CBD and the second purified cannabinoid is CBDV.

In one embodiment of the compositions disclosed herein, the first purified cannabinoid is CBD and the second purified cannabinoid is CBDVA.

In one embodiment of the compositions disclosed herein, the first purified cannabinoid is CBD and the second purified cannabinoid is CBC.

In one embodiment of the compositions disclosed herein, the first purified cannabinoid is CBD and the second purified cannabinoid is CBCA.

In one embodiment of the compositions disclosed herein, the first purified cannabinoid is CBD and the second purified cannabinoid is CBCV.

In one embodiment of the compositions disclosed herein, the first purified cannabinoid is CBD and the second purified cannabinoid is CBCVA.

In one embodiment of the compositions disclosed herein, the first purified cannabinoid is CBD and the second purified cannabinoid is CBG.

In one embodiment of the compositions disclosed herein, the first purified cannabinoid is CBD and the second purified cannabinoid is CBGA.

In one embodiment of the compositions disclosed herein, the first purified cannabinoid is CBD and the second purified cannabinoid is CBGV.

In one embodiment of the compositions disclosed herein, the first purified cannabinoid is CBD and the second purified cannabinoid is CBGVA.

In one embodiment of the compositions disclosed herein, the first purified cannabinoid is CBD and the second purified cannabinoid is CBN.

In one embodiment of the compositions disclosed herein, the first purified cannabinoid is CBD and the second purified cannabinoid is CBNA.

In one embodiment of the compositions disclosed herein, the first purified cannabinoid is CBD and the second purified cannabinoid is CBNV.

In one embodiment of the compositions disclosed herein, the first purified cannabinoid is CBD and the second purified cannabinoid is CBNVA.

In one embodiment of the compositions disclosed herein, the first purified cannabinoid is CBDA and the second purified cannabinoid is CBDV.

In one embodiment of the compositions disclosed herein, the first purified cannabinoid is CBDA and the second purified cannabinoid is CBDVA.

In one embodiment of the compositions disclosed herein, the first purified cannabinoid is CBDA and the second purified cannabinoid is CBC.

In one embodiment of the compositions disclosed herein, the first purified cannabinoid is CBDA and the second purified cannabinoid is CBCA.

In one embodiment of the compositions disclosed herein, the first purified cannabinoid is CBDA and the second purified cannabinoid is CBCV.

In one embodiment of the compositions disclosed herein, the first purified cannabinoid is CBDA and the second purified cannabinoid is CBCVA.

In one embodiment of the compositions disclosed herein, the first purified cannabinoid is CBDA and the second purified cannabinoid is CBG.

In one embodiment of the compositions disclosed herein, the first purified cannabinoid is CBDA and the second purified cannabinoid is CBGA.

In one embodiment of the compositions disclosed herein, the first purified cannabinoid is CBDA and the second purified cannabinoid is CBGV.

In one embodiment of the compositions disclosed herein, the first purified cannabinoid is CBDA and the second purified cannabinoid is CBGVA.

In one embodiment of the compositions disclosed herein, the first purified cannabinoid is CBDA and the second purified cannabinoid is CBN.

In one embodiment of the compositions disclosed herein, the first purified cannabinoid is CBDA and the second purified cannabinoid is CBNA.

In one embodiment of the compositions disclosed herein, the first purified cannabinoid is CBDA and the second purified cannabinoid is CBNV.

In one embodiment of the compositions disclosed herein, the first purified cannabinoid is CBDA and the second purified cannabinoid is CBNVA.

In one embodiment of the compositions disclosed herein, the first purified cannabinoid is CBDV and the second purified cannabinoid is CBDVA.

In one embodiment of the compositions disclosed herein, the first purified cannabinoid is CBDV and the second purified cannabinoid is CBC.

In one embodiment of the compositions disclosed herein, the first purified cannabinoid is CBDV and the second purified cannabinoid is CBCA.

In one embodiment of the compositions disclosed herein, the first purified cannabinoid is CBDV and the second purified cannabinoid is CBCV.

In one embodiment of the compositions disclosed herein, the first purified cannabinoid is CBDV and the second purified cannabinoid is CBCVA.

In one embodiment of the compositions disclosed herein, the first purified cannabinoid is CBDV and the second purified cannabinoid is CBG.

In one embodiment of the compositions disclosed herein, the first purified cannabinoid is CBDV and the second purified cannabinoid is CBGA.

In one embodiment of the compositions disclosed herein, the first purified cannabinoid is CBDV and the second purified cannabinoid is CBGV.

In one embodiment of the compositions disclosed herein, the first purified cannabinoid is CBDV and the second purified cannabinoid is CBGVA.

In one embodiment of the compositions disclosed herein, the first purified cannabinoid is CBDV and the second purified cannabinoid is CBN.

In one embodiment of the compositions disclosed herein, the first purified cannabinoid is CBDV and the second purified cannabinoid is CBNA.

In one embodiment of the compositions disclosed herein, the first purified cannabinoid is CBDV and the second purified cannabinoid is CBNV.

In one embodiment of the compositions disclosed herein, the first purified cannabinoid is CBDV and the second purified cannabinoid is CBNVA.

In one embodiment of the compositions disclosed herein, the first purified cannabinoid is CBDVA and the second purified cannabinoid is CBC.

In one embodiment of the compositions disclosed herein, the first purified cannabinoid is CBDVA and the second purified cannabinoid is CBCA.

In one embodiment of the compositions disclosed herein, the first purified cannabinoid is CBDVA and the second purified cannabinoid is CBCV.

In one embodiment of the compositions disclosed herein, the first purified cannabinoid is CBDVA and the second purified cannabinoid is CBCVA.

In one embodiment of the compositions disclosed herein, the first purified cannabinoid is CBDVA and the second purified cannabinoid is CBG.

In one embodiment of the compositions disclosed herein, the first purified cannabinoid is CBDVA and the second purified cannabinoid is CBGA.

In one embodiment of the compositions disclosed herein, the first purified cannabinoid is CBDVA and the second purified cannabinoid is CBGV.

In one embodiment of the compositions disclosed herein, the first purified cannabinoid is CBDVA and the second purified cannabinoid is CBGVA.

In one embodiment of the compositions disclosed herein, the first purified cannabinoid is CBDVA and the second purified cannabinoid is CBN.

In one embodiment of the compositions disclosed herein, the first purified cannabinoid is CBDVA and the second purified cannabinoid is CBNA.

In one embodiment of the compositions disclosed herein, the first purified cannabinoid is CBDVA and the second purified cannabinoid is CBNV.

In one embodiment of the compositions disclosed herein, the first purified cannabinoid is CBDVA and the second purified cannabinoid is CBNVA.

In one embodiment of the compositions disclosed herein, the first purified cannabinoid is CBC and the second purified cannabinoid is CBCA.

In one embodiment of the compositions disclosed herein, the first purified cannabinoid is CBC and the second purified cannabinoid is CBCV.

In one embodiment of the compositions disclosed herein, the first purified cannabinoid is CBC and the second purified cannabinoid is CBCVA.

In one embodiment of the compositions disclosed herein, the first purified cannabinoid is CBC and the second purified cannabinoid is CBG.

In one embodiment of the compositions disclosed herein, the first purified cannabinoid is CBC and the second purified cannabinoid is CBGA.

In one embodiment of the compositions disclosed herein, the first purified cannabinoid is CBC and the second purified cannabinoid is CBGV.

In one embodiment of the compositions disclosed herein, the first purified cannabinoid is CBC and the second purified cannabinoid is CBGVA.

In one embodiment of the compositions disclosed herein, the first purified cannabinoid is CBC and the second purified cannabinoid is CBN.

In one embodiment of the compositions disclosed herein, the first purified cannabinoid is CBC and the second purified cannabinoid is CBNA.

In one embodiment of the compositions disclosed herein, the first purified cannabinoid is CBC and the second purified cannabinoid is CBNV.

In one embodiment of the compositions disclosed herein, the first purified cannabinoid is CBC and the second purified cannabinoid is CBNVA.

In one embodiment of the compositions disclosed herein, the first purified cannabinoid is CBCA and the second purified cannabinoid is CBCV.

In one embodiment of the compositions disclosed herein, the first purified cannabinoid is CBCA and the second purified cannabinoid is CBCVA.

In one embodiment of the compositions disclosed herein, the first purified cannabinoid is CBCA and the second purified cannabinoid is CBG.

In one embodiment of the compositions disclosed herein, the first purified cannabinoid is CBCA and the second purified cannabinoid is CBGA.

In one embodiment of the compositions disclosed herein, the first purified cannabinoid is CBCA and the second purified cannabinoid is CBGV.

In one embodiment of the compositions disclosed herein, the first purified cannabinoid is CBCA and the second purified cannabinoid is CBGVA.

In one embodiment of the compositions disclosed herein, the first purified cannabinoid is CBCA and the second purified cannabinoid is CBN.

In one embodiment of the compositions disclosed herein, the first purified cannabinoid is CBCA and the second purified cannabinoid is CBNA.

In one embodiment of the compositions disclosed herein, the first purified cannabinoid is CBCA and the second purified cannabinoid is CBNV.

In one embodiment of the compositions disclosed herein, the first purified cannabinoid is CBCA and the second purified cannabinoid is CBNVA.

In one embodiment of the compositions disclosed herein, the first purified cannabinoid is CBCV and the second purified cannabinoid is CBCVA.

In one embodiment of the compositions disclosed herein, the first purified cannabinoid is CBCV and the second purified cannabinoid is CBG.

In one embodiment of the compositions disclosed herein, the first purified cannabinoid is CBCV and the second purified cannabinoid is CBGA.

In one embodiment of the compositions disclosed herein, the first purified cannabinoid is CBCV and the second purified cannabinoid is CBGV.

In one embodiment of the compositions disclosed herein, the first purified cannabinoid is CBCV and the second purified cannabinoid is CBGVA.

In one embodiment of the compositions disclosed herein, the first purified cannabinoid is CBCV and the second purified cannabinoid is CBN.

In one embodiment of the compositions disclosed herein, the first purified cannabinoid is CBCV and the second purified cannabinoid is CBNA.

In one embodiment of the compositions disclosed herein, the first purified cannabinoid is CBCV and the second purified cannabinoid is CBNV.

In one embodiment of the compositions disclosed herein, the first purified cannabinoid is CBCV and the second purified cannabinoid is CBNVA.

In one embodiment of the compositions disclosed herein, the first purified cannabinoid is CBCV and the second purified cannabinoid is CBCA.

In one embodiment of the compositions disclosed herein, the first purified cannabinoid is CBCVA and the second purified cannabinoid is CBG.

In one embodiment of the compositions disclosed herein, the first purified cannabinoid is CBCVA and the second purified cannabinoid is CBGA.

In one embodiment of the compositions disclosed herein, the first purified cannabinoid is CBCVA and the second purified cannabinoid is CBGV.

In one embodiment of the compositions disclosed herein, the first purified cannabinoid is CBCVA and the second purified cannabinoid is CBGVA.

In one embodiment of the compositions disclosed herein, the first purified cannabinoid is CBCVA and the second purified cannabinoid is CBN.

In one embodiment of the compositions disclosed herein, the first purified cannabinoid is CBCVA and the second purified cannabinoid is CBNA.

In one embodiment of the compositions disclosed herein, the first purified cannabinoid is CBCVA and the second purified cannabinoid is CBNV.

In one embodiment of the compositions disclosed herein, the first purified cannabinoid is CBCVA and the second purified cannabinoid is CBNVA.

In one embodiment of the compositions disclosed herein, the first purified cannabinoid is CBG and the second purified cannabinoid is CBGA.

In one embodiment of the compositions disclosed herein, the first purified cannabinoid is CBG and the second purified cannabinoid is CBGV.

In one embodiment of the compositions disclosed herein, the first purified cannabinoid is CBG and the second purified cannabinoid is CBGVA.

In one embodiment of the compositions disclosed herein, the first purified cannabinoid is CBG and the second purified cannabinoid is CBN.

In one embodiment of the compositions disclosed herein, the first purified cannabinoid is CBG and the second purified cannabinoid is CBNA.

In one embodiment of the compositions disclosed herein, the first purified cannabinoid is CBG and the second purified cannabinoid is CBNV.

In one embodiment of the compositions disclosed herein, the first purified cannabinoid is CBG and the second purified cannabinoid is CBNVA.

In one embodiment of the compositions disclosed herein, the first purified cannabinoid is CBGA and the second purified cannabinoid is CBGV.

In one embodiment of the compositions disclosed herein, the first purified cannabinoid is CBGA and the second purified cannabinoid is CBGVA.

In one embodiment of the compositions disclosed herein, the first purified cannabinoid is CBGA and the second purified cannabinoid is CBN.

In one embodiment of the compositions disclosed herein, the first purified cannabinoid is CBGA and the second purified cannabinoid is CBNA.

In one embodiment of the compositions disclosed herein, the first purified cannabinoid is CBGA and the second purified cannabinoid is CBNV.

In one embodiment of the compositions disclosed herein, the first purified cannabinoid is CBGA and the second purified cannabinoid is CBNVA.

In one embodiment of the compositions disclosed herein, the first purified cannabinoid is CBGV and the second purified cannabinoid is CBN.

In one embodiment of the compositions disclosed herein, the first purified cannabinoid is CBGV and the second purified cannabinoid is CBNA.

In one embodiment of the compositions disclosed herein, the first purified cannabinoid is CBGV and the second purified cannabinoid is CBNV.

In one embodiment of the compositions disclosed herein, the first purified cannabinoid is CBGV and the second purified cannabinoid is CBNVA.

In one embodiment of the compositions disclosed herein, the first purified cannabinoid is CBGVA and the second purified cannabinoid is CBN.

In one embodiment of the compositions disclosed herein, the first purified cannabinoid is CBGVA and the second purified cannabinoid is CBNA.

In one embodiment of the compositions disclosed herein, the first purified cannabinoid is CBGVA and the second purified cannabinoid is CBNV.

In one embodiment of the compositions disclosed herein, the first purified cannabinoid is CBGVA and the second purified cannabinoid is CBNVA.

In one embodiment of the compositions disclosed herein, the first purified cannabinoid is CBN and the second purified cannabinoid is CBNA.

In one embodiment of the compositions disclosed herein, the first purified cannabinoid is CBN and the second purified cannabinoid is CBNV.

In one embodiment of the compositions disclosed herein, the first purified cannabinoid is CBN and the second purified cannabinoid is CBNVA.

In one embodiment of the compositions disclosed herein, the first purified cannabinoid is CBNA and the second purified cannabinoid is CBNV.

In one embodiment of the compositions disclosed herein, the first purified cannabinoid is CBNA and the second purified cannabinoid is CBNVA.

In one embodiment of the compositions disclosed herein, the first purified cannabinoid is CBNV and the second purified cannabinoid is CBNVA.

In one embodiment, the above described combinations of a first purified cannabinoid and a second purified cannabinoid include a third purified cannabinoid chosen from THC, D9-THC, D8-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

Accordingly, disclosed herein are new compositions comprising unique combinations of a first purified cannabinoid, a second purified cannabinoid, and a third purified cannabinoid. In one embodiment, the compositions disclosed herein comprise a first purified cannabinoid, a second purified cannabinoid, and a third purified cannabinoid present in non-naturally occurring concentrations. In one embodiment, the compositions disclosed herein comprise a first purified cannabinoid, a second purified cannabinoid, and a third purified cannabinoid present in non-naturally occurring ratios. In one embodiment, the compositions disclosed herein provide particular benefits previously unavailable with naturally occurring cannabinoid profiles, such as those found in plants, harvested flowers, extracts, or conventional products derived from the same.

In one embodiment of the compositions disclosed herein, the ratio of the first purified cannabinoid to the second purified cannabinoid to the third purified cannabinoid is greater than 100:1:1.

In one embodiment of the compositions disclosed herein, the ratio of the first purified cannabinoid to the second purified cannabinoid to the third purified cannabinoid is between 100:1:1 to 10:1:1.

In one embodiment of the compositions disclosed herein, the ratio of the first purified cannabinoid to the second purified cannabinoid to the third purified cannabinoid is between 20:1:1 to 10:1:1.

In one embodiment of the compositions disclosed herein, the ratio of the first purified cannabinoid to the second purified cannabinoid to the third purified cannabinoid is between 10:1:1 to 1:1:1.

In one embodiment of the compositions disclosed herein, the ratio of the first purified cannabinoid to the second purified cannabinoid to the third purified cannabinoid is between 5:1:1 to 1:1:1.

In one embodiment of the compositions disclosed herein, the ratio of the first purified cannabinoid to the second purified cannabinoid to the third purified cannabinoid is between 3:1:1 to 1:1:1.

In one embodiment of the compositions disclosed herein, the ratio of the first purified cannabinoid to the second purified cannabinoid to the third purified cannabinoid is between 1:1:1 to 1:1:10.

In one embodiment of the compositions disclosed herein, the ratio of the first purified cannabinoid to the second purified cannabinoid to the third purified cannabinoid is between 1:1:10 to 1:1:100.

In one embodiment of the compositions disclosed herein, the ratio of the first purified cannabinoid to the second purified cannabinoid to the third purified cannabinoid is between 100:50:1 to 10:5:1.

In one embodiment of the compositions disclosed herein, the ratio of the first purified cannabinoid to the second purified cannabinoid to the third purified cannabinoid is between 50:10:1 to 5:2:1.

In one embodiment of the compositions disclosed herein, the ratio of the first purified cannabinoid to the second purified cannabinoid to the third purified cannabinoid is between 25:15:1 to 1:1:1.

In one embodiment of the compositions disclosed herein, the ratio of the first purified cannabinoid to the second purified cannabinoid to the third purified cannabinoid is between 1:1:1 to 1:5:5.

In one embodiment of the compositions disclosed herein, the ratio of the first purified cannabinoid to the second purified cannabinoid to the third purified cannabinoid is between 1:5:5 to 1:25:100.

In one embodiment of the compositions disclosed herein, the ratio of the first purified cannabinoid to the second purified cannabinoid to the third purified cannabinoid is between 100:100:1 to 10:10:1.

In one embodiment of the compositions disclosed herein, the ratio of the first purified cannabinoid to the second purified cannabinoid to the third purified cannabinoid is between 20:20:1 to 10:10:1.

In one embodiment of the compositions disclosed herein, the ratio of the first purified cannabinoid to the second purified cannabinoid to the third purified cannabinoid is between 10:10:1 to 1:1:1.

In one embodiment of the compositions disclosed herein, the ratio of the first purified cannabinoid to the second purified cannabinoid to the third purified cannabinoid is between 5:5:1 to 1:1:1.

In one embodiment of the compositions disclosed herein, the ratio of the first purified cannabinoid to the second purified cannabinoid to the third purified cannabinoid is between 3:3:1 to 1:1:1.

In one embodiment of the compositions disclosed herein, the ratio of the first purified cannabinoid to the second purified cannabinoid to the third purified cannabinoid is between 1:1:1 to 1:10:10.

In one embodiment of the compositions disclosed herein, the ratio of the first purified cannabinoid to the second purified cannabinoid to the third purified cannabinoid is between 1:10:10 to 1:100:100.

### EXAMPLES

The following examples are provided as non-limiting illustrations of the broader disclosure.

### Example 1

A composition was formulated by combining the following components: purified THC (99 g) and purified Beta-Pinene (0.8 g).

### Example 2

A composition was formulated by combining the following components: purified THC (99 g) and purified Geraniol (0.5 g).

### Example 3

A composition was formulated by combining the following components: purified THC (99 g) and purified p-Cymene (0.5 g).

### Example 4

A composition was formulated by combining the following components: purified THC (99 g) and purified Delta-3-Carene (0.25 g).

### Example 5

A composition was formulated by combining the following components: purified THC (99 g) and purified Camphene (0.375 g).

### Example 6

A composition was formulated by combining the following components: purified THC (99 g) and purified Pulegone (0.3 g).

### Example 7

A composition was formulated by combining the following components: purified THC (99 g) and purified Terpinolene (0.3 g).

### Example 8

A composition was formulated by combining the following components: purified THC (99 g) and purified Alpha-Terpineol (0.2 g).

### Example 9

A composition was formulated by combining the following components: purified THC (98 g) and purified Beta-Myrcene (1.25 g).

### Example 10

A composition was formulated by combining the following components: purified THC (99 g) and purified Alpha-Pinene (0.8 g).

### Example 11

A composition was formulated by combining the following components: purified THC (98 g) and purified Beta-Caryophyllene (1.5 g).

### Example 12

A composition was formulated by combining the following components: purified THC (98 g) and purified Nerolidol (1.5 g).

### Example 13

A composition was formulated by combining the following components: purified THC (97 g) and purified Limonene (1.4 g).

### Example 14

A composition was formulated by combining the following components: purified THC (99 g) and purified Linalool (0.375 g).

### Example 15

A composition was formulated by combining the following components: purified THC (99 g) and purified Beta-Pinene (0.8 g).

### Example 16

A composition was formulated by combining the following components: purified THC (99 g) and purified Eucalyptol (0.5 g).

### Example 17

A composition was formulated by combining the following components: purified THC (99 g) and purified Alpha-Humulene (0.9 g).

### Example 18

A composition was formulated by combining the following components: purified THC (51 g), purified CBD (10 g), purified CBG (4 g), purified Linalool (0.5 g), purified Limonene (0.5 g), and purified Alpha-Humulene (0.5 g).

### Example 19

A composition was formulated by combining the following components: purified THC (50 g), purified THCV (10 g), purified CBG (4 g), purified CBC (0.2 g), purified Beta-Pinene (2 g), purified Beta-Caryophyllene (0.5 g), purified Limonene (1 g), and purified Eucalyptol (0.25 g).

### Example 20

A composition was formulated by combining the following components: purified THC (20 g); purified CBC (10 g); purified CBN (0.2 g); purified THCV (0.6 g); and purified Beta-Pinene (0.8 g).

### Example 21

A composition was formulated by combining the following components: purified THC (51 g); purified CBD (0.2 g); purified CBC (0.2 g); purified CBN (0.2 g); purified THCV (0.6 g); purified Beta-Caryophyllene (1.6 g); and purified Alpha-Terpineol (0.16 g).

### Example 22

A composition was formulated by combining the following components: purified THC (51 g), purified Beta-Pinene (1 g), purified Alpha-Terpineol (0.1 g), and purified Linalool (0.1 g).

### Example 23

A composition was formulated by combining the following components: purified THC (51 g), purified Beta-Pinene (1 g), purified Alpha-Terpineol (0.1 g), purified Linalool (0.1 g), and Polyethylene Glycol (25 g).

### Example 24

A composition was formulated by combining the following components: purified THC (51 g); purified CBD (0.2 g); purified CBC (0.2 g); purified CBN (0.2 g); purified THCV (0.6 g); purified Beta-Caryophyllene (1 g); and purified Linalool (0.1 g).

### Example 25

A composition was formulated by combining the following components: purified THC (51 g), purified Alpha-Pinene (1.6 g) and purified Linalool (0.25 g).

### Example 26

A composition was formulated by combining the following components: purified THC (51 g), purified Alpha-Pinene (1.6 g), purified Linalool (0.25 g), and purified Alpha-Humulene (0.6 g).

### Example 27

A composition was formulated combining the following components: purified THC (51 g), purified Linalool (0.5 g), purified Alpha-Pinene (0.75 g), and purified Eucalyptol (0.75 g).

### Example 28

A composition was formulated combining the following components: purified THC (51 g), purified Linalool (0.5 g), purified Alpha-Pinene (0.75 g), purified Eucalyptol (0.75 g), and purified Nerolidol (1.5 g).

### Example 29

A composition was formulated combining the following components: purified THC (51 g), purified Linalool (0.5 g), purified Alpha-Pinene (1.6 g), purified Eucalyptol (0.75 g), and Polyethylene Glycol (25 g).

### Example 30

A composition was formulated combining the following components: purified THC (51 g), purified Linalool (0.5 g), purified Alpha-Pinene (1.6 g), purified Eucalyptol (0.75 g), purified Terpinolene (0.25 g), and Polyethylene Glycol (25 g).

### Example 31

A composition was formulated combining the following components: purified THC (51 g), purified Beta-Caryophyllene (1 g), purified Alpha-Humulene (1 g), and Polyethylene Glycol (25 g).

### Example 32

A composition was formulated combining the following components: purified THC (51 g), purified Beta-Caryophyllene (1 g), purified Alpha-Humulene (2 g), and Polyethylene Glycol (25 g).

### Example 33

A composition was formulated combining the following components: purified THC (51 g), purified Beta-Caryophyllene (2 g), purified Alpha-Humulene (2 g), and Polyethylene Glycol (25 g).

### Example 34

A composition was formulated combining the following components: purified THC (75 g) and purified THCV (5 g).

### Example 35

A composition was formulated combining the following components: purified THC (50 g) and purified CBD (10 g).

### Example 36

A composition was formulated combining the following components: purified THC (50 g) and purified CBG (5 g).

### Example 37

A composition was formulated combining the following components: purified THC (75 g) and purified CBC (15 g).

### Example 38

A composition was formulated combining the following components: purified THC (75 g) and purified CBN (10 g).

### Example 39

The clinical effects of terpene concentrations were measured by comparing the differences in response measured between (a) administering a purified cannabinoid alone and (b) administering the same purified cannabinoid in concert with a purified terpene. Each of the following variables were systematically evaluated: cannabinoid compound(s) present; terpene compounds(s) present; and their absolute and relative concentrations.

In each observational study, each participant was given a formulation having a particular chemical composition pursuant to the guidelines above. Subjective feelings were scored on a Visual Analogue Scale (VAS), in web-based survey questions, 60 minutes after subjects start to vape a sample. Each VAS question asked subjects to click on a horizontal bar at a location that reflected the degree to which their feelings were described by adjectives positioned at either end of the bar. Clicking at the left end of the bar returned a score of zero whereas clicking at the right end returned a score of 10. “Unipolar” questions asked subjects to report the strength of a singular feeling on a zero (not at all) to 10 (extremely) scale (Bowdle reference). “Bipolar” questions were also scored on a 0 to 10 scale. For bipolar questions, clicking in the middle of the bar (score of 5) meant that any given subject reported neutrality with respect to the adjectives positioned at either end of the bar.

Subjective feelings scores reported for each VAS survey question were averaged, and mean values were compared between base oil, and low and high terpene-enriched samples. Sample sizes in these experiments were low, yielding suggestive interpretations with regard to feelings elicited by terpene enrichment. Hence, the most prominent feelings elicited by terpene enrichment were identified according to conservative criteria: 1) when mean scores trended toward a given VAS adjective as terpene enrichment moved from zero to high percentage, and 2) when mean scores changed by at least 50% as they trended from low to high score.

The following table illustrates how clinical results may be organized to compare trends in particular feelings when studying terpene enrichment.

| | | Mean value from | | |
|---|---|---|---|---|
| | | VAS survey question | | |
| Terpene | Elicited | Base | Low | High |
| enrichment | subjective feeling | oil | enrichment | enrichment |
| Eucalyptol | Well-coordinated | 4.7 | 3.7 | 3.0 |
| | More Anxious (U) | 0.7 | 1.7 | 2.0 |
| | More Stoned (U) | 1.7 | 3.0 | 3.0 |
| | Upset | 2.3 | 3.7 | 3.7 |
| | Hungry | 3.3 | 3.7 | 6.0 |
| | Troubled | 7.7 | 6.3 | 4.7 |
| | Tense | 7.0 | 6.3 | 4.7 |
| | Sad | 2.7 | 2.7 | 4.7 |
| | Clearheaded | 5.3 | 6.3 | 7.0 |
| Beta-Pinene | More Anxious (U) | 1.0 | 2.5 | 7.5 |
| | More Paranoid (U) | 0.5 | 2.5 | 7.5 |
| | Gloomy | 8.0 | 6.0 | 2.5 |
| | Quiet | 7.5 | 6.5 | 2.5 |
| | Sluggish | 2.0 | 3.5 | 7.5 |
| | Moistmouth | 7.5 | 6.5 | 2.5 |
| | Uninspired | 1.5 | 3.5 | 7.5 |
| | Cautious | 7.5 | 6.0 | 2.5 |
| | Uninventive | 2.0 | 4.0 | 7.5 |
| | Upset | 1.0 | 2.5 | 7.5 |
| | Restless | 9.0 | 7.5 | 2.5 |
| | Drowsy | 3.0 | 3.5 | 6.0 |
| | Discontented | 1.5 | 2.5 | 7.5 |
| | Sad | 2.0 | 2.5 | 6.5 |
| | Bored | 1.0 | 2.5 | 6.5 |
| | Agitated | 2.0 | 2.5 | 7.5 |
| Alpha-Pinene | Attentive | 0.0 | 3.0 | 5.0 |
| Alpha-Humulene | More Stoned (U) | 0 | 61 | 74 |
| | More High (U) | 0 | 45 | 60 |
| | Passing of time is | 0 | 32 | 51 |
| | altered more (U) | | | |
| | Heavenly | 51 | 36 | 17 |
| | Interested | 61 | 32 | 17 |
| | Excited | 39 | 43 | 73 |
| | Headhigh | 51 | 51 | 16 |
| Limonene | Drowsy | 6 | 23 | 30 |
| | Drymouth | 46 | 67 | 84 |
| Linalool | Interested | 55 | 49 | 31 |
| | Happy | 19 | 28 | 29 |
| | Gregarious | 38 | 53 | 60 |
| | Creative | 58 | 41 | 34 |
| | In the moment | 46 | 56 | 67 |
| Nerolidol | Less Stoned (U) | 75 | 68 | 54 |
| | Less Anxious (U) | 67 | 42 | 28 |
| | Less Paranoid (U) | 68 | 38 | 28 |
| | Discontented | 23 | 28 | 40 |
| | Mindful | 37 | 52 | 61 |
| Beta- | Passing of time is | 77 | 54 | 44 |
| Caryophyllene | altered (U) | | | |
| | Lazy | 12 | 23 | 55 |
| | Disjointed | 19 | 46 | 68 |
| | Confused | 34 | 45 | 55 |
| | Dreamy | 28 | 38 | 67 |
| | Bored | 16 | 34 | 43 |
| | Sad | 10 | 28 | 35 |
| | Calm | 64 | 62 | 33 |
| | Discontented | 23 | 28 | 40 |
| | Unaware | 29 | 34 | 55 |
| | Chill | 25 | 34 | 69 |
| | Bodyhigh | 11 | 38 | 60 |
| | Depressed | 21 | 32 | 36 |
| | Uncreative | 34 | 38 | 67 |
| | Lethargic | 67 | 60 | 39 |
| Beta-Myrcene | More Anxious (U) | 6 | 10 | 26 |
| | More Drowsy (U) | 20 | 30 | 44 |
| | More Paranoid (U) | 1 | 10 | 34 |
| | Disjointed | 24 | 37 | 40 |
| | Drymouth | 31 | 38 | 47 |
| | Upset | 28 | 38 | 43 |
| | Depressed | 30 | 36 | 46 |
| Alpha-Terpineol | Intensity of sound | 27 | 15 | 10 |
| | changes less (U) | | | |
| | Scattered | 64 | 57 | 29 |
| | Confused | 30 | 50 | 57 |
| | Drowsy | 32 | 49 | 65 |
| | Unaware | 28 | 44 | 54 |
| | Unproductive | 14 | 44 | 59 |
| | Chill | 29 | 57 | 81 |
| | Bodyhigh | 24 | 34 | 68 |
| | Lethargic | 63 | 44 | 31 |
| | Inattentive | 70 | 51 | 42 |
| | Dull | 30 | 55 | 61 |
| | Sluggish | 33 | 56 | 61 |
| | Absent | 24 | 50 | 60 |
| | Unsociable | 38 | 59 | 66 |
| Terpinolene | Intensity of colors | 28 | 41 | 54 |
| | changed more (U) | | | |
| | Heavenly | 42 | 31 | 26 |
| | Creative | 51 | 37 | 25 |
| | Talkative | 76 | 53 | 42 |
| | Constructive | 62 | 41 | 37 |
| | Uplifted | 50 | 36 | 28 |
| | Inventive | 48 | 34 | 29 |
| | Lively | 70 | 48 | 39 |
| | More High (U) | 35 | 49 | 57 |
| | The passing of | 14 | 17 | 34 |
| | time is altered (U) | | | |
| | Intensity of sound | 19 | 22 | 40 |
| | changed (U) | | | |
| | Bored | 25 | 31 | 44 |
| | Discontented | 17 | 21 | 33 |
| | Clearheaded | 24 | 38 | 40 |
| | Bodyhigh | 26 | 41 | 50 |
| Pulegone | Centered | 47 | 34 | 31 |
| Camphene | Excited | 26 | 39 | 44 |
| | Gregarious | 32 | 52 | 61 |
| | Wild | 21 | 27 | 36 |
| | Social | 66 | 48 | 43 |
| Delta-3-Carene | More Drowsy (U) | 12 | 14 | 36 |
| | Drymouth | 47 | 60 | 72 |
| | Worried | 24 | 29 | 38 |
| Geraniol | Less Paranoid (U) | 23 | 8 | 3 |
| | Interested | 45 | 37 | 27 |
| | Worried | 16 | 22 | 27 |
| | Active | 50 | 43 | 32 |
| | Headhigh | 39 | 34 | 20 |
| | Restless | 70 | 69 | 45 |
| p-Cymene | Less Anxious (U) | 22 | 16 | 15 |
| | More Drowsy (U) | 23 | 24 | 41 |
| | Interested | 41 | 36 | 28 |
| | Calm | 37 | 31 | 15 |
| | Laidback | 33 | 28 | 18 |
| | Depressed | 32 | 37 | 51 |
| | Mellow | 44 | 52 | 68 |
| | Lethargic | 64 | 58 | 37 |
| | Unsocial | 32 | 34 | 57 |

### Example 40

Cannabis plant material was cooled to −20 C for 24 hours by placing the cannabis plant material in a freezer. The cold plant material was then triturated twice with cold ethanol (2 mL of ethanol at −20 C per gram of cannabis plant material) by agitating the mixture plant material and ethanol for 3 minutes, then decanting the liquid from the mixture. Portions of decanted ethanol were combined and cooled at −20 C for 24 hours. The precipitate was then removed by gravity filtration. The ethanol was removed from the filtrate by evaporation, leaving an amber oil. The oil was dissolved in ethanol (10-100 mg/mL). This solution was injected into a Supercritical Fluid Chromatograph equipped with an Ethyl Pyridine column. Pure fractions were collected and analyzed by LC-MS, Multiple Reaction Monitoring transitions, full scan detection, photodiode array detection, and Evaporative Light Scattering Detection to assess purity.

### Example 41

Cannabis plant material was cooled to −20 C for 24 hours by placing the cannabis plant material in a freezer. The cooled plant material was ground into particles having a diameter of approximately 2 mm. The particles were transferred into a cylinder and connected to a supercritical fluid extraction device for extracting cannabinoids and flavonoids. The extracted material was collected as an amber oil. The oil was dissolved in ethanol (10-100 mg/mL). This solution was injected into a Supercritical Fluid Chromatograph equipped with an Ethyl Pyridine column. Pure fractions were collected and analyzed by LC-MS, Multiple Reaction Monitoring transitions, full scan detection, photodiode array detection, and Evaporative Light Scattering Detection to assess purity.

### Example 42

A composition was formulated by combining the following components: THCV (1 g); CBG (1 g); CBC (1 g); THC (2 g); and CBD (4 g); b-Caryophllene (0.2); Linalool (0.005 g); Limonene (0.001 g); Pilegone (0.005 g); and Hurnulene (0.005 g).

### Example 43

A composition was formulated by combining the following components: THCV (1 g); CBG (1 g); CBC (2 g); THC (1 g); and CBD (2 g); 1,8-Cineol (0.005 g); a-Pinene (0.005 g); and Limonene (0.001 g).

### Example 44

A composition was formulated by combining the following components: CBG (1 g); CBC (2 g); THC (4 g); and CBD (1 g); 1,8-Cineol (0.0025 g); a-Pinene (0.0025 g); b-Mycrene (0.001 g); and Limonene (0.001 g).

### Example 45

A composition was formulated by combining the following components: THCV (2 g); CBG (1 g); CBC (1 g); THC (3 g); and CBD (1 g); b-Caryophyllene (0.0025 g); Linalool (0.001 g); 1,8-Cineol (0.0025 g); b-Mycrene (0.0025 g); Limonene (0.001 g); and Pilegone (0.001 g).

### Example 46

A composition was formulated by combining the following components: CBG (1 g); THC (5 g); and CBD (1 g); b-Caryophyllene (0.05 g); Linalool (0.01 g); b-Mycrene (0.001 g); Limonene (0.001 g); Pilegone (0.01 g); and Humulene (0.01 g).

These examples are meant to be non-limiting and serve only to illustrate possible embodiments of the formulations disclosed herein.

Although the present invention herein has been described with reference to various exemplary embodiments, it is to be understood that these embodiments are merely illustrative of the principles and applications of the present invention. Those having skill in the art would recognize that various modifications to the exemplary embodiments may be made, without departing from the scope of the invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s)

1.-21. (canceled)

22. A composition comprising:
a purified cannabinoid; and
Theanine.

23. The composition of claim 22, wherein the purified cannabinoid is selected from Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C₄ (CBD-C₄), Cannabidivarinic Acid (CBCVA), Cannabidivarin (CBDV), Cannabidiorcol (CBD-C₁), Tetrahydrocannabinolic acid A (THCA-A), Tetrahydrocannabinolic acid B (THCA-B), Tetrahydrocannabinol (THC), Tetrahydrocannabinolic acid C₄ (THCA-C₄), Tetrahydrocannabinol C₄ (THC-C₁), Tetrahydrocannabivarinic acid (THCVA), Tetrahydrocannabivarin (THCV), Tetrahydrocannabiorcolic acid (THCA-C₁), Tetrahydrocannabiorcol (THC-C₁), Delta-7-cis-iso-tetrahydrocannabivarin, Δ⁸-tetrahydrocannabinolic acid (Δ⁸-THCA), Δ⁸-tetrahydrocannabinol (Δ⁸-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C₄ (CBN-C₄), Cannabivarin (CB V), Cannabino-C₂ (CBN-C₂), Cannabiorcol (CBN-C₁), Cannabinodiol (CBND), Cannabinodivarin (CBDV), Cannabitriol (CBT), 10-Ethoxy-9-hydroxy-Δ^{6a}-tetrahydrocannabinol, 8,9-Dihydroxy-Δ^{6a(10a)}-tetrahydrocannabinol (8,9-Di-OH-CBT-Cs), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ^{6a(10a)}-tetrahydrocannabinol (OTHC), Δ⁹-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC) and isocannabinoids.

24. The composition of claim 1, wherein the purified cannabinoid is selected from Tetrahydrocannabinol (THC), Tetrahydrocannabivarin (THCV), Cannabichromene (CBC), Cannabichromanon (CBCN), Cannabidiol (CBD), Cannabielsoin (CBE), Cannabidivarin (CBDV), Cannbifuran (CBF), Cannabigerol (CBG), Cannabicyclol (CBL), Cannabinol (CBN), Cannabinodiol (CBND), Cannabitriol (CBT), Cannabivarin (CBV), and isocanabinoids.

25. The composition of claim 22, wherein the purified cannabinoid is Cannabidiol (CBD).

26. The composition of claim 22, wherein the purified cannabinoid is Cannabigerol (CBG).

27. The composition of claim 22, further comprising a purified terpene.

28. The composition of claim 27, wherein the purified terpene is selected from 7,8-dihydroionone, Acetanisole, Acetic Acid, Acetyl Cedrene, Anethole, Anisole, Benzaldehyde, Bergamotene (α-cis-Bergamotene) (α-trans-Bergamotene), Bisabolol (β-Bisabolol), Borneol, Butanoic/Butyric Acid, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol, Cryptone, Curcumene (α-Curcumene) (γ-Curcumene), Decanal, Dehydrovomifoliol, Diallyl Di sulfide, Dihydroactinidiolide, Dimethyl Disulfide, Eicosane/Icosane, Elemene (β-Elemene), Estragole, Ethyl acetate, Ethyl Cinnamate, Ethyl maltol, Eucalyptol/1,8-Cineole, Eudesmol (α-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1(10),11-diene, Guaiacol, Guaiene (α-Guaiene), Gurjunene (α-Gurjunene), Herniarin, Hexanaldehyde, Hexanoic Acid, Humulene (α-Humulene) (β-Humulene), Ionol (3-oxo-α-ionol) (β-IonooI), lonone (α-lonone) (β-lonone), Ipsdienol, Isoamyl acetate, Isoamyl Alcohol, Isoamyl Formate, Isoborneol, Isomyrcenol, Isopulegol, isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, Terpin hydrate, Terpineol, Terpene-4-ol, α-Terpinene, γ-Terpinene, Terpinolene, Thiophenol, Thujone, Thymol, α-Tocopherol, Tonka Undecanone, Undecanal, Valeraldehyde/Pentanal, Verdoxan, α-Ylangene, Umbelliferone, and Vanillin.

29. The composition of claim 27, wherein the purified terpene is selected from Alpha-terpineol, Bergamotene (α-cis-Bergamotene) (α-trans-Bergamotene), Bisabolol (β-Bisabolol), Borneol, Camphor, Capsaicin, Carvacrol, Catyophyllene (β-Caryophyllene), Caryophyllene oxide, Cineole, Cinnamaldehyde (α-amyl-Cinnamaldehyde) (α-hexyl-Cinnamaldehyde), Cinnamyl Alcohol, Citronellal, Citronellol, Ethyl, Cinnamate, Eucalyptol/1,8-Cineole, Farnesol, Fenchol (β-Fenchol), Geranyl acetate, Germacrene B, Guaiene (α-Guaiene), Humulene (α-Humulene) (β-Humulene), lonol (3-oxo-α-ionol) (β-lonol), lonone (α-lonone) (β-lonone), Isoamyl acetate, Isoprene, Kahweol, Laevo-Carvone, Lavandulol, Limonene, Linalool, Linalyl acetate, Luteolin, Lycopene, Menthol, Myrcene (β-Myrcene), Nerol, Nerolidol, Neryl acetate, P-cymene, Phytol, Pinene, Pristimerin, Retinol, Terpin hydrate, Terpineol, Terpinol-4-ol, Terpinolene, Thujone, Thymol, and Vanillin.

30. The composition of claim 27, wherein the purified terpene is Linalool, Limonene, or a combination thereof.

31. The composition of claim 27, wherein the purified terpene is Linalool and Limonene.

32. A composition comprising:
a purified cannabinoid selected from Tetrahydrocannabinol (THC), Tetrahydrocannabivarin (THCV), Cannabichromene (CBC), Cannabichromanon (CBCN), Cannabidiol (CBD), Cannabielsoin (CBE), Cannabidivarin (CBDV), Cannbifuran (CBF), Cannabigerol (CBG), Cannabicyclol (CBL), Cannabinol (CBN), Cannabinodiol (CBND), Cannabitriol (CBT), Cannabivarin (CBV), and isocanabinoids;
a purified terpene; and
Theanine.

33. The composition of claim 32, wherein the purified cannabinoid is Cannabidiol (CBD).

34. The composition of claim 32, wherein the purified cannabinoid is Cannabigerol (CBG).

35. The composition of claim 32, wherein the purified terpene is selected from 7,8-dihydroionone, Acetanisole, Acetic Acid, Acetyl Cedrene, Anethole, Anisole, Benzaldehyde, Bergamotene (α-cis-Bergamotene) (α-trans-Bergamotene), Bisabolol (β-Bisabolol), Borneol, Butanoic/Butyric Acid, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol Cryptone, Curcumene (α-Curcumene) (γ-Curcumene), Decanal, Dehydrovomifoliol, Diallyl Disulfide, Dihydroactinidiolide, Dimethyl Disulfide, Eicosane/lcosane, Elemene (β-Elemene), Estragole, Ethyl acetate, Ethyl Cinnamate, Ethyl maltol, Eucalyptol/1,8-Cineole, Eudesmol (α-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1(10),11-diene, Guaiacol, Guaiene (α-Guaiene), Gurjunene (α-Gurjunene), Herniarin, Hexanaldehyde, Hexanoic Acid, Humulene (α-Humulene) (β-Humulene), lonol (3-oxo-α-ionol) (β-IonoI), lonone (α-lonone) (β-lonone), Ipsdienol, Isoamyl acetate, Isoamyl Alcohol, Isoamyl Formate, Isoborneol, Isomyrcenol, Isopulegol Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, Terpin hydrate, Terpineol, Terpine-4-ol, α-Terpinene, γ-Terpinene, Terpinolene, Thiophenol, Thujone, Thymol, α-Tocopherol, Tonka Undecanone, Undecanal, Valeraldehyde/Pentanal, Verdoxan, α-Ylangene, Umbelliferone, and Vanillin.

36. The composition of claim 32, wherein the purified terpene is selected from Alpha-terpineol, Bergamotene (α-cis-Bergamotene) (α-trans-Bergamotene), Bisabolol (β-Bisabolol), Borneol, Camphor, Capsaicin, Carvacrol, Caryophyllene (β-Caryophyllene), Caryophyllene oxide, Cineole, Cinnamaldehyde (α-amyl-Cinnamaldehyde) (α-hexyl-Cinnamaldehyde), Cinnamyl Alcohol, Citronellal, Citronellol, Ethyl, Cinnamate, Eucalyptol/1,8-Cineole, Farnesol, Fenchol (β-Fenchol), Geranyl acetate, Germacrene B, Guaiene (α-Guaiene), Humulene (α-Humulene) (β-Humulene), lonol (3-oxo-α-ionol) (β-lonol), Ionone (α-lonone) (β-lonone), Isoamyl acetate, Isoprene, Kahweol, Laevo-Carvone, Lavandulol, Limonene, Linalool, Linalyl acetate, Luteolin, Lycopene, Menthol, Myrcene (β-Myrcene), Neral, Nerolidol, Neryl acetate, P-cymene, Phytol, Pinene, Pristimerin, Retinol, Terpin hydrate, Terpineol, Terpinol-4-ol, Terpinolene, Thujone, Thymol, and Vanillin.

37. The composition of claim 32, wherein the purified terpene is Linalool, Limonene, or a combination thereof.

38. The composition of claim 32, wherein the purified terpene is Linalool and Limonene.

39. A composition comprising:
a purified cannabinoid selected from Tetrahydrocannabinol (THC Tetrahydrocannabivarin (THCV), Cannabichromene (CBC), Cannabichromanon (CBCN), Cannabidiol (CBD), Cannabielsoin (CBE), Cannabidivarin (CBDV), Cannbifuran (CBF), Cannabigerol (CBG), Cannabicyclol (CBL), Cannabinol (CBN), Cannabinodiol (CBND), Cannabitriol (CBT), Cannabivarin (CBV), and isocanabinoids;
a purified terpene selected from Linalool, Limonene, or a combination thereof; and
Theanine.

40. The composition of claim 39, wherein the purified cannabinoid is Cannabidiol (CBD).

41. The composition of claim 39, wherein the purified cannabinoid is Cannabigerol (CBG).
